(19) United States
(12) Patent Application Publication
(10) Pub. No.: US 2007/0166699 A1
(43) Pub. Date: Jul. 19, 2007

(54) Combination of anti-hpv-16 and 18 antibodies and uses thereof

(75) Inventors: Werner Paul Zwerschke, Innsbruck (AT); Pidder Jansen-Duerr, Innsbruck (AT); Marc Fiedler, Birgitz (AT); Andreas Laich, Innsbruck (AT); Barbara Fitzky, Axams (AT)

Correspondence Address: FOLEY AND LARDNER LLP;SUITE 500, 3000 K STREET NW, WASHINGTON, DC, 20007, US

(73) Assignee: AMYNON BIOTECH GmbH

(21) Appl. No.: 10/572,408

(22) PCT Filed: Sep. 17, 2004
(86) PCT No.: PCT/EP04/10484

§ 371 Date: Mar. 16, 2006

(30) Foreign Application Priority Data
Sep. 17, 2003 (EP) 03020564.5

(51) Int. Cl. C12Q 1/70; A61K 39/42; C07K 16/10

(52) U.S. Cl. 435/5; 424/159.1; 530/388.3

(57) ABSTRACT

The present invention relates to a combination of antibodies comprising (a) an anti-HPV-16 E7 antibody obtainable by (i) eliciting an in vivo humoral response against HPV-16 E7 protein or a fragment thereof in a goat; and (ii) affinity-purifying antibodies as obtained in the eliciting-step (i) and (b) an anti-HPV-18 E7 antibody. Additionally, in another aspect the present invention relates to methods for producing said combination of antibodies. Furthermore, the invention provides for the use of the combination of antibodies or for the use of an anti-HPV-16 E7 antibody obtainable as mentioned above for the preparation of a diagnostic composition for the (immuno-) histological detection of high risk HPV in a biological sample. Additionally, the invention relates to diagnostic compositions comprising said combination of antibodies or said anti-HPV-16 E7 antibody obtainable as mentioned above as well as to methods for producing said diagnostic compositions. The invention also provides for kits comprising said combination of antibodies of the present invention or said anti-HPV-16 E7 antibody obtainable as mentioned above or a diagnostic composition of the invention and discloses in vitro methods and uses for the detection of E7 protein of high risk HPV such as HPV-16, HPV-18, HPV-31, HPV-33, HPV-35, HPV-39, HPV-45, HPV-52, HPV-56, HPV-58 and/or HPV-59 indicating a sexually transmittable disease or cancer, in particular cervical cancer, breast cancer, prostate cancer, head and neck cancer, penile cancer or anogenital cancer by using the described combination of antibodies.

The present invention relates to a combination of antibodies comprising (a) an anti-HPV-16 E7 antibody obtainable by (i) eliciting an in vivo humoral response against HPV-16 E7 protein or a fragment thereof in a goat; and (ii) affinity-purifying antibodies as obtained in the eliciting-step (i) and (b) an anti-HPV-18 E7 antibody. Preferably, said HPV-16 E7 protein or a fragment thereof is highly purified. Additionally, in another aspect the present invention relates to methods for producing said combination of antibodies. Furthermore, the invention provides for the use of the combination of antibodies or for the use of an anti-HPV-16 E7 antibody obtainable as mentioned above for the preparation of a diagnostic composition for the (immuno-) histological detection of high risk HPV in a biological sample. Additionally, the invention relates to diagnostic compositions comprising said combination of antibodies or said anti-HPV-16 E7 antibody obtainable as mentioned above as well as to methods for producing said diagnostic compositions. The invention also provides for kits comprising said combination of antibodies of the present invention or said anti-HPV-16 E7 antibody obtainable as mentioned above or a diagnostic composition of the invention and discloses in vitro methods and uses for the detection of E7 protein of high risk HPV such as HPV-16, HPV-18, HPV-31, HPV-33, HPV-35, HPV-39, HPV-45, HPV-52, HPV-56, HPV-58 and/or HPV-59 indicating a sexually transmittable disease or cancer, in particular cervical cancer, breast cancer, prostate cancer, head and neck cancer, penile cancer or anogenital cancer by using the described combination of antibodies.

Despite an intensive screening program, cervical cancer is one of the most predominant neoplastic diseases in women with a world-wide incidence second only to breast cancer (Walboomers, 1999). A major etiological factor in the genesis of cervical carcinoma is the infection by human papillomaviruses (HPVs), which are small DNA viruses that infect epithelial cells of either the skin or mucosa. Of the almost 100 different types of HPV that have been characterized to date, approximately two dozen specifically infect genital and oral mucosa (reviewed in zur Hausen, 2000).

On the basis of epidemiological and biochemical data, HPVs are subdivided into two groups. Genital HPVs of the high-risk group most commonly HPV-16, 18, 31, 33, 35, 39, 45, 52, 56, 58, 59, 68, 69, 73, 81 cause cervical cancer and other anogenital cancers while papillomaviruses of the low-risk group most frequently HPV-1, 6, 11 cause inter alia benign genital warts (for review, see Howley, 1996).

HPV is the primary cause of cervical cancer in all cases. Human Papillomavirus infection is a very common sexually transmitted infection, with more than 30 genital types; however, only 10-15 types were detected in cervical cancers. Therefore this types are defined as oncogenic types, cancer-assosciated or high risk HPV types. These comprise HPV type 16, 18, 31, 33, 35, 39, 45, 52, 56, 58 and 59. Approximately 80% of cervical cancer worldwide are associated with only four types (16, 18, 31 and 45) with small variations between different countries. In another 15%, of said cancers types 33, 35 and 52 are detected.

PCR based studies have shown that more than 99% of invasive cervical cancers world-wide contain high risk HPVs (Walboomers, 1999); however, malignant progression occurs only in a small subset of infected patients and is typically slow (reviewed in Alexander and Phelps, 2000). Most cervical dysplasia represent squamous cell carcinoma and the main diagnostic tool to detect cervical dysplastic cells is still based on cytological screening using Pap-smear analysis introduced in 1943 by Papanicolaou (1942) (for review, see Koss, 1989; Meijer and Walboomers, 2000). Although Pap-smear analysis has proven highly effective it is difficult to standardise, which is reflected by a high error rate of approximately 30% including false-positives and/or false-negatives (Walboomers, 1995; Clavel, 1999; Renshaw et al., 2001). Despite the introduction of mass screening programs, the best of which have dropped the mortality rates by 70%, incidence of cervical cancer in the United States has been increasing by about 3% a year since 1986 in spite of an intensification of the rate of screening (Larsen, 1994).

Since it is medical general knowledge that cervical cancer arises as consequence of persistent high-risk papillomavirus infections (reviewed in zur Hausen, 2000), the problem could be addressed by the introduction of HPV tests into screening programmes for better identification of patients at risk. At present, for clinical applications, PCR and the hybrid capture analysis, both are DNA-detecting methods, are only useful to a limited extend (reviewed in Milde-Langosch, 2000). Milde-Langosch furthermore teaches that, besides the molecular biological methods, some antibodies against early HPV-proteins are sold (for example by Santa Cruz Biotechnology or Dianova) but these antibodies are, in comparison to these molecular biological techniques even less sensitive in (immuno)-histochemical analysis.

The major disadvantage of the above discussed molecular biology methods systems is, that they do only allow to detect viral infection, however, about 5-30% of the normal female population harbours these viruses and only very few of these develop clinically relevant lesions (von Knebel Doeberitz, 2001). In accordance with this consideration, a high rate of transient and asymptomatic HPV infections was found especially among young woman (Schiffman and Brinton, 1995). Given the low incidence of cervical cancer, it may not be useful to apply HPV detection for cervical cancer screening in this age group. Moreover, the PCR based screening systems, although highly sensitive, are not widely applied for the reason that HPV DNA will be detected in a wide range of normal cytological smears resulting in a high rate of false positive amplification (reviewed in Trofatter, 1997).

It is well established that the expression of E6 and E7, in epithelial stem cells of the mucosa, is required to initiate and maintain cervical carcinogenesis (for recent review, see Mantovani and Banks, 2001; Münger, 2001). Thus, a promising way to improve the screening programs could be to measure the expression of the E6 and E7 oncoprotein which initiate in a long term process neoplastic transformation in few of the HPV harbouring cells. Since these viral proteins are not expressed in normal cervical squamous epithelia, screening for high risk E7 over-expressing cells allows to specifically identify dysplastic lesions. Moreover, progression of pre-neoplastic lesions to invasive cervical cancers is often associated with a continuous enhanced expression of the E6 and E7 oncoprotein (Schwarz, 1985; Francis, 2000). Similar to these considerations, Klaes (2001) monitored the overexpression of the cyclin-dependent kinase inhibitor p16 (INK4a), a gene which is upregulated in response to E7, as a marker for dysplastic and neoplastic epithelial cells of the cervix uteri.

However, p16 (INK4A) is only one of several genes which are upregulated in response to E7 (for review, see McMurray, 2001) and upregulation of p16 (INK4A) expression is not necessary for E7 induced malignant transformation (Giarre, 2001). Furthermore, in view of its central role as tumorsuppressor and cell cycle inhibitory protein p16 (INK4A) is upregulated by several other, growth suppressing, stimuli, thus p16 (INK4a) is for example well known as target of senescence-inducing pathways (for review, see Bringold and Serrano, 2000). Consequently upregulation of p16 (INK4A) might not necessarily reflect the activity of the E7 oncoprotein.

Furthermore, some studies have speculated on the prevalence of anogenital types of human papillomavirus in prostate cancer and benign prostate hypertrophy. Interestingly, the prevalence of an HPV, in particular HPV-16 infection in prostate carcinogenesis is highly disputed. Cuzick (1995, Cancer Sarr. 23, 91-95) reviewed earlier reports on this issue and stress that it is unlikely that common anogenital papillomaviruses have an important role in prostate carcinogenesis. Several studies have linked the presence of HPV16 DNA to a risk for developing prostate cancer (e.g. Moyret-Lalle, 1997, Int. J. Cancer 64, 125-129; Jerris, 1997 Urology 50, 150-156 or Suzuki, 1996, Prostate 28, 318-324), yet, these studies did not provide for a conclusive relationship between prostate cancerogenesis and HPV-16 activities and/or the expression of HPV16 proteins. Wideroff (1996, Prostate 28, 117-123) even teaches that HPV infection is not a significant risk factor for prostate cancer and Anderson (1997, J. Med. Virol. 52, 8-13) confirms the teaching that HPV16 and closely related types are unlikely initiations of prostate cancer. Similarly Noda (1998, Urol. Res. 26, 165-169) suggests that HPV is not a causal factor for prostatic cancer or benign prostatic hyperplasia, and Stickler (1998, Cancer 82, 1118-1125 and 1998, Eur. J. Canc. Prev. 7, 305-313) comes to the conclusion that HPV is not associated with prostate carcinomas. Even if Serth (1998) analysed by single-tube quantitative, competitive PCR samples from prostate cancers and indicates that in accordance with this DNA-detection method, HPV16 might contribute to the development of a subset of prostate cancers (Serth (1999), Canc. Res. 59, 823-825), another study of the same year (Saad (1999), Can. J. Urol. 6, 834-838) could not detect HPV DNA in fresh tissue from patients undergoing radical prostatectomy for prostate cancer. Accordingly, the role of HPVs, in particular HPV16, in prostate cancer remains controversial and elusive.

Several sets of monoclonal antibodies against the HPV-16 E7 oncoprotein or HPV-16 E7 derived peptides have been produced (Sato, 1989; Tindle, 1990; Selvey, 1992; Stacey, 1994; Fujikawa, 1994; Zatsepina, 1997) and commercial preparations are also available (Zymed Laboratories, San Francisco, Calif., USA; Santa Cruz Biotechnology, Santa Cruz, Calif., USA). No antibody, however, was reported as sufficient in sensitivity and specificity to recognise HPV-16 E7 neither in cytological smears nor in paraffin embedded sections from biopsies of cervical cancer patients; see Milde-Langosch, 1999. Di Lonardo (2001, Arch Viral 146, 117-125) has produced egg yolk antibodies as well as rabbit antibodies against E7 oncogenic protein of HPV16. Di Lonardo (2001), loc. cit. stresses that some commercial preparations of anti-E7 antibodies are available, but they suffer severe disadvantages and are not suitable for diagnostic purposes. Yet, the data provided by Di Lonardo (2001) are not conclusive since merely the hen antibodies were able to localize HPV-16 E7 in a cultured cell line and in a SIL (Squameous Interepithelial lesion) biopsy. However, Di Lonardo (2001) loc. cit. also teaches that the rabbit antibodies are not able to detect E7 in immunocytochemistry and stressed that the generated hen antibodies were in a number of cases unable to detect E7 protein in immunostainings of cervical lesions.

Documentation that certain high-risk types of human papillomavirus (HPV) are necessary in the etiology of cervical cancer does it make conceivable to introduce HPV based tests into screening programmes for better identification of patients at risk. Thus it is well established that the expression of the E6 and E7 genes, in epithelial stem cells of the mucosa is required to initiate and maintain cervical carcinogenesis. For these reasons, a promising way to improve the screening programmes could be the measurement of the expression level of the E6 or E7 oncoprotein which initiate, in a long term process, neoplastic transformation in few of the HPV harbouring cells. These viral genes are not expressed in normal cervical squamous epithelia and the progression of pre-neoplastic lesions to invasive cervical cancers is presumably associated with a continuous enhanced expression of the E6 and E7 oncoproteins. For there reasons screening for cells overexpressing high risk E7 oncoprotein may allow to specifically grade dysplastic lesions. In view of the above, means for the reliable detection of the E7 protein of a multitude of HPV strains, such as HPV-16, HPV-18, HPV-31, HPV-45 belonging to the group of HPV which are responsible for the above-described diseases is highly desirable. However, EP 0 299 354 only describes an E7III antibody which is said to recognize the E7 protein of HPV-16 and, due to cross-reactivity, also the E7 protein of HPV-18. HPV-16 and HPV-18 are, however, only two representatives of the large group of HPV which can cause the diseases described herein. EP 0 375 555 describes an anti-HPV 16-antibody and EP 0 256 321 provides an anti-HPV-18 antibody. Yet, there are so far no means available which allow a full coverage screen for those HPV which represent the clinically most relevant group responsible for, e.g., cervix cancer, breast cancer or prostate cancer.

Accordingly, there is a need in the art for antibodies and/or antibody combinations which are able to detect E7 protein of a multitude of HPVs or clinically relevant subgroups of HPVs and which would allow for the detection of the malignant state of a cancerous cell or for the detection of a sexually transmittable disease.

Thus, the technical problem underlying the present invention is to comply with the need described above. This technical problem is solved by the provision of the embodiments defined in the claims.

Accordingly, the present invention provides a combination of antibodies comprising
(a) an anti-HPV-16 E7 antibody obtainable by
   (i) eliciting an in vivo humoral response against HPV-16 E7 protein or a fragment thereof in a goat; and
   (ii) affinity-purifying antibodies as obtained in the eliciting-step (i); and
(b) an anti-HPV-18 E7 antibody.

The combination of antibodies of the present invention is a simple tool for detecting a multitude of different HPV strains and can be used as a tool in the (immuno)-histochemical detection of HPV infection, preferably infection with high risk HPV and/or in cancer diagnostic. Inter alia, this is because it is known that expression of E7 protein of HPV in epithelial stem cells of the mucosa is required to initiate and maintain cervical carcinogenesis and that continuous enhanced expression of E7 oncoprotein takes place in the progression of pre-neoplastic lesions to invasive cervical cancer. Thus, detection of the E7 protein of HPV(s), preferably high risk HPV(s) is indicative for infection with HPV, preferably high risk HPV in cancer diagnostic. The term “high risk HPV” when used in the context of the present invention means HPV which are the causative agents for more than 98% of all clinically relevant tumors and/or found preferably in cervical and anogenital carcinomas, but not in benign warts of the anogenital region of patients. Most preferably, said high risk HPV embrace HPV-16, HPV-18, HPV-31 and/or HPV-45. Particularly preferred, said high risk HPV embrace HPV-16, HPV-18, HPV-31, HPV-33, HPV-35, HPV-39, HPV-45, HPV-52, HPV-56, HPV-58 and/or HPV-59. Preferably said high risk HPV embrace HPV-16, HPV-18, HPV-31, HPV-33, HPV-35, HPV-39, HPV-45, HPV-52, HPV-56, HPV-58, HPV-59, HPV-68, HPV-69, HPV-73 and/or HPV-82. The combination of antibodies as provided herein is particularly useful in the detection of the high risk HPV-types 16, 18, 31 and 45. Accordingly, the present invention provides for an antibody-combination which does not only detect HPV-16 and HPV-18, but also other high risk HPVs as defined herein and known in the art. The combination of antibodies provided herein is particularly useful in the parallel detection of HPV-16 and HPV-18 in particular in combination with HPV-31, HPV-33, HPV-35, HPV-39, HPV-45, HPV-52, HPV-56, HPV-58 and/or HPV-59. In a preferred embodiment, the HPV-high risk viruses/viral infections to be detected are selected from the group consisting of HPV-31, HPV-33, HPV-35, HPV-45 and HPV-52 in combination with HPV-16 and/or HPV-18. As documented in the appended examples, the antibody combination provided herewith is not only capable of detecting HPV-16 and HPV-18 but also further high risk HPVs and in particular also HPV-31. The antibody combination provided herein is, accordingly, also capable of detecting HPV-31 (E7) or an infection with HPV-31. Accordingly, it is envisaged that the combination of antibodies of the present invention detects E7 protein(s) of one or more of said high risk HPV or any possible combination thereof. The combination of antibodies provided herein is, inter alia, useful in the direct measurement of expressed E7 oncoprotein in biological samples, of the HPV, preferably high risk HPV listed above, for example in cells or lysates of Pap-smears cervix biopsies, prostate biopsies, in particular from fine needle aspiration biopsies. Accordingly, the combination of antibodies of the present invention is particularly useful in immunochemistry/immunohistochemistry methods as documented in the appended examples. The inventive combination may be employed to detect E7 of HPV-16 and HPV-18 in particular in combination with E7 of further HPV E7 proteins as documented herein. Therefore, the antibody combination provided herein is capable to detect, in particular in immunohistochemistry and immunocytochemistry HPV-E7 from a plurality of high risk HPVs and notably from HPV-16 and HPV-18.

It was surprisingly found that the anti-HPV-16 E7 antibodies produced according to step (i) described above are, in contrast to antibodies of the prior art, capable of reliably detecting expressed E7 of various HPV, preferably high risk HPV like HPV-16, 31, 33, 35, 39, 45, 52, 56, 58 and/or 59. This is because it was unexpectedly found that eliciting an in vivo humoral response against highly purified HPV-16 E7 protein or fragment thereof in a goat according to steps (i) and (ii) described herein leads to the generation of antibodies which recognize not only E7 protein of HPV-16, but also recognize at least the E7 protein of HPV-31, HPV-33, HPV-35, HPV-39, HPV-45, HPV-52, HPV-56 and/or HPV-59 as shown in the appended Examples. As shown in the examples even if the antibody as generated in the methods as described in step (i) above detects the HPV-16 E7 band in Western Blots as a single band, said antibody is still and unexpectedly capable of also recognizing native E7, e.g. folded of other high risk HPVs, for example in immunohistochemistry and/or immunocytochemistry methods. Without being bound by theory, the HPV-16 E7 antibody provided herein appears to specifically recognize (besides E7 from HPV-16) other native (e.g. folded) E7 proteins from other high risk HPVs in cellular context. Said “cellular context” relates to the potential detection of high risk HPV-E7 in cells, tissues and/or organs, in particular in fixed as well as non-fixed material and most preferably in immunohistochemical and/or immunocytochemical methods (like immunofluorescence or immunostain methods on cells, tissues or organs). Without being bound by theory, the binding of the antibodies to HPV E7 proteins depends on small linear epitopes of the HPV-16 and HPV-18 E7 protein and, accordingly, on conformational epitopes of the native folded protein.

The antiHPV-16 and -18 E7 antibodies as employed in the combination of antibodies of the present invention recognize only the linear epitopes of HPV-16 and HPV-18 E7 protein in, e.g. Western Blot using denatured E7, but they do, preferably, not crossreact with denatured linearized E7 protein of the other high risk HPV types.

The antibodies used herein and documented in the experimental part are capable of recognizing further none denatured high risk E7 proteins in transient transfected cells (IF) and patients material (IHC). In these cells, the E7 proteins are expressed and correctly folded. The fixation of the material before staining does not linearize/denature the proteins but does stabilise the conformation.

The antibodies to be used in the combination of the invention also recognize the E7 protein of other high risk HPV as described above, e.g. the E7 protein of HPV-31 or HPV-56 as shown in the examples.

However, the so obtained antibodies do not recognize E7 protein of low risk HPV, such as HPV-11 as shown in the appended Examples. Preferably, these antibodies do also not recognize the E7 protein of HPV-1 and more preferably these antibodies do not recognize the E7 protein of HPV-6. HPV-6 and/or HPV-11 cause inter alia benign genital warts in the anogenital region and may thus contaminate Pap smears when taken from a patient. Accordingly, it is envisaged that the combination of antibodies of the present invention advantageously detects E7 protein of high risk HPV. In accordance with the present invention the term “low risk HPV” embraces HPV-1, HPV-6 and/or HPV-11. HPV-6 and/or HPV-11 are mostly found in the anogenital region of patients, for example in genital warts, but not in cervix carcinomas HPV-1 causes, for example, skin warts.

The above mentioned elicitation of an in vivo humoral response against HPV-16 E7 protein or a fragment thereof is most preferably carried out employing a highly-purified HPV-16 E7 protein or a highly-purified fragment thereof. Corresponding highly-purified HPV-16 E7 (or a fragment thereof) may be obtained by methods provided below and illustrated in the appended examples.

It is clear for the person skilled in the art that the combination of antibodies provided herein is useful in particular in diagnostic medical settings. Accordingly, said antibody combination may be used to determine whether a given sample is HPV-16-, HPV-18-positive and whether or not said sample is also positive for other high risk HPVs, in particular HPV-31, HPV-33, HPV-35, HPV-39, HPV-45, HPV-52, HPV-56, HPV-58 and/or HPV-59. The combination is particularly useful in determining whether an infection with HPV-31, HPV-35, HPV-39, HPV-45 and/or HPV-59, particularly an infection with HPV-31 exists in a given sample.

The unexpected finding that the anti-HPV-16 E7 antibody recognizes many of the E7 proteins of HPV strains which belong to the high risk HPV group, but not HPV-11 of the low risk group of HPV, enables now the recognition of a multitude of high risk HPVs which are the causative agent of sexually transmittable disease or cancer as described supra by applying the combination of antibodies of the present invention. By supplementing the anti-HPV-16 E7 antibodies generated according to the methods of the present invention with an anti-HPV-18 E7 antibody as described supra provides coverage of the recognition of all clinically important high risk HPV responsible for more than 98% of cervical carcinomas which has so far not been possible.

The term “anti-HPV-16 E7 antibody” as employed herein refers to an antibody, a plurality of antibodies and/or a serum comprising such antibodies which is/are able to bind to, interact with or detect particularly the E7 oncoprotein of HPV16. As documented in the appended examples said “anti-HPV-16 E7 antibody” is in particular capable of detecting HPV-16 E7 protein in histological or cell-samples. Said antibody is in particular functional in immunofluorescence methods (and similar methods). Said antibody is particularly useful in binding to/interacting with and/or detecting HPV-16 E7 protein in biological, in particular in histological samples and in probes, like pap-smear probes. An anti-HPV-16 E7 antibody produced according to steps (i) and (ii) described above, shows the characteristics of binding also E7 proteins of other HPVs, in particular high risk HPV such as HPV-31, HPV-33, HPV-35, HPV-39, HPV-45, HPV-52, HPV-56, HPV-58 and/or HPV-59 or a fragment thereof and/or not E7 protein of HPV-11 which belongs to the low risk HPV. The antibody directed against HPV-16 E7 of the invention is capable to also detect the high risk HPVs mentioned above and in particular HPV-31. The term “anti-HPV-16 E7 antibody” also relates to a purified serum, i.e. a purified polyclonal serum. The antibody molecule is preferably a full immunoglobulin, like an IgG, IgA, IgM, IgD, IgE. The term “antibody” as used in this context of this invention also relates to a mixture of individual immunoglobulins. Furthermore, it is envisaged that the antibody/antibody molecule is a fragment of an antibody, like an F(ab), F(abc), Fv Fab′ or F(ab)₂. Furthermore, the term “antibody” as employed in the invention also relates to derivatives of the antibodies which display the same specificity as the described antibodies. Such derivatives may, inter alia, comprise chimeric antibodies or single-chain constructs. Yet, most preferably, said “anti-HPV-16 E7 antibody” relates to a serum, more preferably a polyclonal serum and most preferably to a purified (polyclonal) serum. The antibody/serum is obtainable, and preferably obtained, by the method described herein and illustrated in the appended Examples. The anti-HPV-16 E7 antibody is preferably comprised in a combination of antibodies as described infra.

The term “anti-HPV-18 E7 antibody” as used in the context of the present invention refers to an antibody, a plurality of antibodies and/or a serum comprising such antibodies which is/are able to bind to, interact with or detect particularly the E7 oncoprotein of HPV-18 or a fragment thereof. However, it is also envisaged that the anti-HPV-18 E7 antibody may recognize due to cross-reactivity the E7 protein(s) of other HPV, preferably E7 protein(s) of high risk HPV and preferably not E7 protein(s) of low risk HPV. Particularly preferred, said anti-HPV-18 E7 antibody may also recognize E7 protein of HPV-16, HPV-31, HPV-35 and/or not E7 proteins of the low risk HPV-1, HPV-6 and/or HPV-11. Corresponding examples are given in the rabbit serum provided in the appended examples or in an anti-HPV-45 E7 antibody which is also capable of binding to, interacting with or detecting E7 of HPV-18. Accordingly, an “anti-HPV-18 E7 antibody” to be employed in the inventive combination of antibodies provided herewith is not limited to an antibody that was raised against HPV-18 E7 but also comprises antibodies which were raised against other HPV-E7 proteins and fragments thereof and is capable of binding to/interacting with E7 of HPV-18. As mentioned above, corresponding examples are the rabbit serum as provided in the examples (recognizing E7 of HPV-16 and HPV-18) or the antibody raised against-HPV-45 (provided in the examples) which also recognize native (e.g. in a cellular context expressed, native and/or folded) E7 of HPV-18. Accordingly, the term “anti-HPV-18 E7 antibody” also comprises antibodies which were raised against E7 proteins (or fragments thereof) of other (high risk) HPVs, but which show cross-reactivities with HPV-18 E7, in particular in immunohistological and immunocytochemical methods and settings provided herein. The term “antibody” also relates to a purified serum, i.e. a purified polyclonal serum. The antibody molecule is preferably a full immunoglobulin, like an IgG, IgA, IgM, IgD, IgE, IgY (for example in yolk derived antibodies). The term “antibody” as used in this context of this invention has been described supra and applies to the anti-HPV-18 E7 antibody, mutatis mutandis. Particularly preferred, said “anti-HPV-18 E7 antibody” is a monoclonal or polyclonal antibody or preferably a CDR-grafted antibody, humanized antibody, human antibody, single chain antibody or chimeric antibody or diabody. Yet, more preferably, said “anti-HPV-18 E7 antibody” relates to a serum, more preferably a polyclonal serum and most preferably to a purified (polyclonal) serum. The antibody/serum is obtainable, and preferably obtained, by the method described herein and illustrated in the appended Examples. Preferably, said anti-HPV-18 E7 antibody is comprised in a combination of antibodies described infra. An example of an anti-HPV-18 E7 antibody is described in EP 0 256 321. Yet, more preferably the (rabbit) antibody described in PCT/EP03/02990 which recognizes, besides E7 of HPV-16, also the E7 protein of HPV-18 as demonstrated in the appended Examples herein is comprised in the combination of antibodies of the present invention.

The term “eliciting an in vivo humoral response in a goat” relates to the provocation of an immune response in a goat, in particular the provocation of an antibody response to HPV-16 E7 or a fragment thereof. A preferred goat species is “Saanen breed goat”. Said antibody response comprises primary as well as secondary antibody responses to the antigenic challenge with HPV-16 E7 or a fragment thereof. The term “eliciting an in vivo humoral response”, accordingly, relates to the provocation of an immune reaction involving the production of antibodies directed towards the antigen, namely HPV-16 E7 or a fragment thereof.

When used in the context of the present invention the term “combination of antibodies” means a mixture of the anti-HPV-16 E7 antibodies obtained in step (a) described supra and an anti-HPV-18 E7 antibody as described supra. Said mixture may be preexisting or may be generated immediately before or during applying the combination of antibodies of the present invention. Thus, it is envisaged that the anti-HPV-16 E7 antibodies are applied at first and then the anti-HPV-18 E7 antibody or vice versa. Alternatively, both antibodies are applied together. Accordingly, the antibodies of the combination of antibodies may be stored lyophilisated or in aqueous/liquid solutions either separately or together, for example, in the form of stock-solutions which the person skilled in the art is aware of diluting appropriately according to the intended use or in the form of “ready-to-use” solutions. Said solutions may be buffered according to methods known in the art. The combination of antibodies of the present invention may comprise further ingredients such as stabilizing proteins, for example BSA or glycin or preservatives known in the art.

The term “combination of antibodies comprising an anti-HPV-16 E7 antibody (obtainable in a goat) and an anti-HPV-18 antibody” does also encompass an antibody-combination wherein said anti-HPV-16 E7 antibody was generated in a goat and wherein said anti-HPV-18 E7 antibody was also generated in a goat. Accordingly, said term also comprises, inter alia, a serum obtained in goat by double-immunization with HPV-16 E7 (or a fragment thereof) and HPV-18 E7 (or a fragment thereof). Said double-immunization may be carried out by eliciting an in vivo human response against HPV-16 E7 and HPV-18 E7 in said goat. Said double-immunization may be carried out by administering to said goat HPV-16 E7 (or a fragment thereof) and HPV-18 E7 (or a fragment thereof). Said administration of E7 of HPV-16 and HPV-18 may be carried out sequentially or it may comprise an administration of both antigens at the same time.

As mentioned above, in a most preferred embodiment the HPV-16 E7 protein or a fragment thereof to be used for immunization of (a) goat(s) is “highly purified”.

The term “highly purified HPV-16 E7 protein or a fragment thereof” relates to an isolated HPV-16 E7 protein or fragment thereof, which has been purified to a purity level of at least 95%, more preferably of at least 96%, even more preferably of at least 97%, particularly preferred of at least 98% and most preferably of at least 99% purity. The purity of HPV-16 E7 protein may be confirmed by methods known in the art, preferably by densitometrical analysis as illustrated in Verdoliva (2000, J. Chormatogr. B. Biomed. Sci. Appl. 279, 233-242), Aboagye-Mathiesen (1992, Prep. Biochem. 22: 105-121) and most preferably as described in the appended Examples. It is preferred that said “highly purified HPV-16 E7 protein or a fragment thereof” is purified in order to obtain the corresponding protein or fragment thereof in NMR-grade (further information is given in the appended examples). In context of this invention, the term “highly purified HPV-16 E7 protein” relates to a purified protein E7-preparation which is at least 90%, more preferably at least 95%, more preferably at least 98% most preferably at least 99% pure. Accordingly, the highly, purified HPV-16 E7 preparation to be employed in the immunization protocols described herein comprises preferably less than 5% contaminating, unrelated proteins or protein fragments. Most preferably, said preparation comprises less than 2% contaminating, unrelated proteins or protein fragments. Purity of the highly-purified E7 preparation may be measured by methods known in the art which comprise gel stainings (in particular silver stains of SDS-PAGE followed by densitometric analysis) NMR-measurements or mass spectroscopy (MS). The purity of E7 protein or fragments thereof is in accordance with this invention, most preferably measured by analyzing samples comprising said E7 or (a) fragment(s) thereof by SDS-PAGE, followed by conventional silver staining and densitometric analysis. Corresponding protocols are detailed in the appended Examples. In accordance with this invention “highly purified E7 preparations” to be employed in immunization protocols do not comprise any contaminating, unrelated proteins. According to NMR-analysis the highly-purified E7 protein (or immunogenic fragment(s) thereof) is present in a native, partially unfolded structure. Corresponding Examples for such a purification is given in the appended Examples. In a most preferred embodiment, the highly-purified E7-preparation is a “native, highly purified HPV-16 E7 protein” as defined herein below. It is in particular preferred that said “native, highly purified HPV-16 E7 protein” is a full length protein, comprising preferably 98 amino acids. Most preferably, said anti HPV-16 E7 protein is encoded by a nucleic acid molecule as shown in SEQ ID NO: 1 or is a protein as shown in SEQ ID NO: 2.

The native, highly purified HPV-16 E7 protein or a fragment thereof is preferably recombinantly produced and, most preferably, said protein or fragment thereof lacks further modifications like additional tags, like His-tags or GST-tags. Corresponding HPV-16 E7 sequences are known in the art and also depicted in SEQ ID NO: 1 (nucleic acid sequence encoding HPV-16 E7) and SEQ ID NO: 2 (amino acid sequence of HPV-16 E7).

In accordance with this invention, the term “native, highly purified HPV-16 E7 protein” relates to a protein which is correctly folded or relates to a stretch/fragment of said protein which is correctly folded and which is soluble, preferably highly soluble. As such, the protein is purified from E. coli under native conditions and it is not required to unfold/refold the protein by chaotropic agents, such as urea or guanidinium hydrochloride. It is in particular preferred that the native HPV-16 E7 protein comprises equivalent amounts of zinc, which is required for correct secondary structure of the E7 protein. It is of note that the term “native HPV-16 E7 protein” corresponds to the term “native, highly purified HPV-16 E7” in context of this invention and also comprises naturally occurring variants of HPV-16 E7 protein. Such variants are known in the art, as, inter alia, described by Sang Song (1997, Gynecologic Oncology 66, 275-281) or by Ku (2001), Dis. Of Colon and Rectum 44, 236-242. The person skilled in the art is easily in a position to determine the folding status of said “native HPV-16 E7 protein”, e.g. by CD analysis provided, inter alia, in the appended Examples. It is envisaged, in accordance with this invention, that a native, highly purified HPV-16 E7 protein (or an immunogenic fragment thereof) is to be employed in the immunization protocols provided herein in its native, partially unfolded structure. Therefore, in purified and soluble form said E7 protein (or its immunogenic fragment) comprises, at least partially secondary structures like α-helices, β-sheets and turns and coils. In a most preferred embodiment the E7-protein to be employed in the immunization protocols provided herein comprises 7 to 8% α-helices, 45 to 47% β-sheets, 3 to 5% turns and 40 to 43% coils. The terms “β-sheet”, “α-helix”, “turn” and “coil” are very well known in the art and, inter alia, described in Brandon/Tooze (1991), “Introduction to Protein Structure”; Garland Publishing Inc., London. The HPV-16 E7 fragment to be employed in immunization protocols in accordance with this invention preferably comprise 6 to 9% α-helices, 43 to 47% β-sheets, 1 to 7% turns and/or 38 to 45% coils. In accordance with this invention it was surprisingly found that E7 protein can recombinantly be expressed and obtained in a soluble, native form as described herein. The use of highly purified recombinant E7 proteins in immunization protocols led surprisingly to high quality antibodies specific for said E7 protein. In contrast to antibodies of the prior art, the antibodies of the present invention (raised against highly purified, soluble and, preferably, native E7) are capable of specifically detecting E7 in immunobiological/immunohistochemical samples, like smears. As documented in the appended Examples, prior art antibodies fail to provide for specific detection means for E7. Moreover, prior art anti-HPV-16 E7 antibodies fail to detect E7 proteins of more than two E7 proteins of HPV. For example, the E7III antibody described in EP 0 299 354 is only said to detect E7 of HPV-16 and to cross-react with E7 of HPV-18, however, does not recognize E7 protein of, for example, HPV-31, HPV-33, HPV-35, HPV-39, HPV-45, HPV-52, HPV-58 and/or HPV-59 like the anti-HPV-16 E7 antibody of the combination of antibodies of the present invention.

Notably, the antibodies of the present invention preferably recognize the E7 proteins described herein which have been used for immunization, for example in a Western Blot. Yet, said anti-H PV-16 E7 antibodies recognize E7 proteins of other HPV types which have not been used for immunization, for example, when being applied in immunohistochemistry or immunocytochemical methods on (biological) tissues, cells and/or organs. The antibodies as comprised in the combination of the present invention, accordingly, are particularly useful in detecting folded E7 proteins in cellular context. Corresponding examples of detections in cells, tissues and smears are given in the experimental part.

The term “fragment of HPV-16 E7 protein” as used herein relates to fragments of a length of at least 40, at least 50, more preferably at least 60, even more preferably at least 65 amino acid residues of the native HPV-16 E7 protein. The amino acid sequence of HPV-16 E7 and of corresponding variants is known in the art and published in Seedorf (1987, EMBO J. 6, 139-144), Sang Song (1997, loc. cit.) or Ku (2001, loc. cit.). Preferably, said fragment comprises at least the stretch of amino acids 33 to 98 of HPV-16 E7 as disclosed in Seedorf (loc. cit.). Even more preferably, however, is an E7-protein fragment that comprises at least amino acids 1 to 70 of HPV-16 E7 as disclosed in Seedorf (loc. cit.). Also preferred is the HPV-16 E7 as encoded by the nucleic acid molecules shown in SEQ ID NO: 1 or comprising the amino acid sequence as shown in SEQ ID NO: 2. As mentioned herein, also fragments, i.e. antigenic fragments of said E7 may be employed.

Preferably, the recombinantly produced HPV-16 E7 protein or its fragment is expressed in a prokaryotic host, preferably in E. coli. Yet, also other expression systems are envisaged which comprise:
Bacterial expression systems, for example, pET System, P_{L} Expression System, pCAL Vectors, pGEX Vectors, PRO Bacterial Expression System or Yeast expression systems, like pESP Vectors, pESC Vectors, Pichia Expression system, YES Vector collection, SpECTRA S. pombe Expression System, pYD1 System or Insect expression systems, like BacPAK System, Bac-to-Bac Baculovirus Expression System, Bac-N-Blue Baculovirus Expression System, DES: The Drosophila Expression System, Insectselect System or Viral expression systems, like AdEasy Adenoviral Vector System, MV Helper-Free System ViraPort Retroviral Gene Expression System, Adeno-X Expression System, PLXSN System or Mammalian expression systems, like pMSG System, pCMV Script, pCI, Creator Gene Cloning & Expression System, Tet-On; Tet-Off Gene Expression System.

As illustrated in the appended example, preferably, said highly purified HPV-16 E7 protein or a fragment thereof is purified by a combination of ion exchange chromatography and gel filtration and said purification may further comprise, prior to ion exchange chromatography and gel filtration, a protein precipitation step.

Ion exchange chromatography is known to the artisan and ion exchange media comprise, but are not limited to Mini beads Q, Source 15 Q, Source 30 Q, Sepharose High Performance Q, Sepharose Fast Flow Q, Sepharose XL Q, Sepharose Big Beads Q, DEAE, Streamline DEAE (all from Amersham Biosciences, Vienna, Austria), DEAE-cellulose, QA-cellulose, CM-cellulose, SE-cellulose, DE-52 (Whatman, Kent, England) or Agarose based ion exchangers. Most preferably a Mono QHR 10/10 column (Amersham Biosciences, Vienna, Austria) is employed. It is of note that also normal gravity flow or FPLC systems may be employed.

Gel filtration systems and media are also known to the skilled artisan which comprise Superdex peptide, Superdex 30, Superdex 200, Superose 6, Superose 12, Sephacryl, Sphadex (all from Amersham Biosciences, Vienna, Austria), Biogel P, Agarose-gel, Fracto-gel or Ultro-gel. A most preferred gel filtration system, also employed in the appended Examples, is a HiLoad 16/60 Superdex 75 gel filtration column.

Protein precipitation techniques comprise, inter alia, Dextran sulphate-, Polyethylene glycol (PEG) 4000-8000-, Acetone-, Protamine sulphate-, Streptomycin sulphate-, pH-shift-precipitations. Preferably, said protein precipitation is carried out by ammonium sulfate precipitation. More preferably a 30%, most preferably a 38% saturated (NH₄)SO₄-solution is employed.

Accordingly, an example of such a purification method is a three step purification comprising: 1. protein precipitation, 2. ion exchange chromatography 3. gel filtration. As illustrated in appended example 2, which comprises more details, this precipitation method may preferably be carried out by an ammonium sulfate precipitation using 30% saturated (NH₄)₂SO₄ solution, most preferably a 38% saturated (NH₄)SO₄-solution, an ion exchange chromatography using a Mono Q HR10/10 column (Amersham Biosciences, Vienna, Austria) and a gel filtration using a HiLoad 16/60 Superdex 75 gel filtration column (Amersham Biosciences, Vienna, Austria)

It is also envisaged that, as step before the protein precipitation or in addition to the protein precipitation, the crude cell lysate is centrifuged, for example at 70 000×g for 1 hour.

As mentioned herein above, the antibodies obtained after eliciting an immune response against the highly purified HPV-16 E7 (preferably native, highly purified HPV-16 E7) or a fragment thereof are further purified, in particular affinity purified. Preferably, said affinity purification of the obtained antibodies is carried out over immobilized HPV-16 E7 protein or a fragment thereof. Most preferably, said HPV-16 E7 protein or a fragment thereof is immobilized on PVDF membranes, nitrocellulose, sepharose, agarose, DEAE-cellulose or DEAE. As illustrated in the appended Examples, one possibility of affinity purifying the HPV-16 E7 or a fragment thereof comprises the immobilization of HPV-16 E7 or said fragment on PVDF membranes or affinity columns containing CNBr-activated Sepharose 4B. Immobilized HPV-16 E7 protein is incubated with the polyclonal HPV-16 E7 antiserum, washed, and the affinity purified antibodies are eluted by an acid gradient from the immobilized HPV-16 E7 protein. Corresponding protocols are illustrated in the appended Examples.

The elution of bound anti-HPV-16 E7 antibodies may be carried out by methods known in the art which, inter alia, comprise acid gradients or salt gradients.

In a particularly preferred embodiment, the HPV-16 E7 protein is prepared as described in Examples 1 and 2, appended hereto.

The anti-HPV-18 E7 antibody of the combination of antibodies is preferably a polyclonal or monoclonal antibody. Said anti-HPV-18 E7 antibody is preferably an antibody directed against E7 from HPV-18 or a fragment thereof. E7 of HPV-18 is known in the art and also corresponds to a protein as encoded by the nucleic acid molecule as shown in SEQ ID NO: 3 (or a fragment thereof) or as shown in SEQ ID NO: 4 (or a fragment thereof).

Techniques for the production of antibodies are well known in the art and described, e.g. in Harlow and Lane “Antibodies, A Laboratory Manual”, CSH Press, Cold Spring Harbor, 1988. For the preparation of monoclonal antibodies, any technique which provides antibodies produced by continuous cell line cultures can be used. Examples for such techniques include the hybridoma technique (Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Techniques describing the production of single chain antibodies (e.g., U.S. Pat. No. 4,946,778) can be adapted to produce single chain antibodies to the E7 protein of HPV-18. Furthermore, transgenic mice may be used to express humanized antibodies directed against said HPV-18 E7 protein.

Particularly preferred, the polyclonal anti-HPV-18 E7 antibody is derived from a non-human animal selected from the group consisting of rat, mouse, guinea pig, chicken, duck, sheep, horse, goat, pig, cattle and donkey. Yet, in a most preferred embodiment, the polyclonal anti-HPV-18 E7 antibody is obtainable by
(i) eliciting an in vivo humoral response against highly purified anti-HPV-16 E7 protein or fragment thereof in a rabbit; and
(ii) affinity-purifying antibodies as obtained in the eliciting step (i).
As is demonstrated in the appended Examples, generation of a polyclonal anti-HPV-18 E7 antibody is surprisingly achieved by eliciting an in vivo humoral response against highly purified anti-HPV-16 E7 protein or fragment thereof in a rabbit. This is because the so obtainable polyclonal antibodies show cross-reactivity against other E7 proteins of high risk HPV, in particular against E7 of HPV-18, HPV-31 and/or HPV-35 which is demonstrated in the appended Examples.

It is of particular note that the combination of antibodies of the invention are also capable of detecting E7 of high risk HPV in fixed material, e.g. in formaldehyde-fixed biological samples. The detection is also possible in paraffin- or frozen sections of biological samples and tissue. As documented in the appended Examples, the described combination of antibodies may be employed in (immuno)-histological techniques, like immunostainings of biological tissue (e.g. cervix tissue) or in probes derived from fine needle aspiration biopsies (e.g. prostate tissue).

Accordingly, the present invention provides for improved diagnostic tools for the detection of an high risk HPV infection, for example for the detection of an high risk HPV induced ongoing tumor disease. The combination of antibodies of the present invention is particular useful for the detection of E7 protein of high risk HPV in Pap-smears.

The detection of, e.g., enhanced E7 oncoprotein expression level by the provided combination of antibodies allows to identify pre-neoplastic lesions with a particularly high risk for malignant progression and invasive cancers on histological probes and/or in cytological smears. This helps to improve current limitations in cancer screening, diagnosis, and therapy control, in particular in cervical and prostate cancer. The described combination of antibodies provides for useful tools in the classification of sexually transmitted diseases or of cancer. Furthermore, the antibodies of the combination of antibodies of the present invention against highly purified HPV-16 E7 protein or a fragment thereof recognize E7 protein of a multitude of HPVs as described above in neoplastic cells derived from, e.g., cervical smears, in paraffin- or in frozen-sections from biopsies of patients which has so far not been possible. Thus, these antibodies have major diagnostic potential as markers of malignant transformation in, inter alia, carcinogenesis, e.g. cervical carcinogenesis or prostate carcinogenesis.

The antibodies of the combination of antibodies of the present invention described herein have major advantages over the antibodies of the prior art, e.g. commercially available antibodies as, inter alia, provided by Santa Cruz Biotechnologies or Zymed Laboratories. In contrast to antibodies and antibody-reagents provided by the prior art, the antibodies/sera of the combination of antibodies described herein are highly specific and do not provide for high number of “false-positive” signals, i.e. of a “positive” immunobiochemical signal in samples or cells which are high risk HPV negative or which do not express the E7 protein of high risk HPV or a fragment thereof. Additionally, the herein described antibodies of the combination of antibodies are not only highly specific but do also not provide for a high number of “false-negative” immunobiochemical signals. Moreover, the combination of antibodies of the present invention is capable of detecting E7 protein of high risk HPV which are assumed to be responsible for more than 98% of all cervical carcinomas. As illustrated in the appended Examples the antibodies of the combination of antibodies of the invention may be, inter alia, tested for this reliability in transfection studies. For example, cultured cells, preferably human U2-OS cells may be transfected with a vector heterologously expressing E7, for example a vector which provides for CMV-driven expression of, for example, HPV-16 E7, HPV-18, HPV-31 or HPV-45. As a negative control, further U2-OS-cells may be transfected with an expression vector which does not express said E7 protein. “False positive” signals are evaluated by the amount of cells which are not transfected with the E7-expressing vector, but which, nevertheless, give a positive signal in immunobiological screenings, e.g. immunofluorescence microscopy. Preferably, less than 15%, more preferably less than 10%, even more preferably less than 5%, most preferably none of the cultured cells which do not (transiently or permanently) express E7-proteins (“negative control cells”) are stained by the antibody described herein. “False negative” signals are evaluated by the amount of cells which are positively transfected with the E7-expressing vector or which are positively infected by E7-expressing HPV, but which give a negative signal in immunobiological screenings, e.g. immunofluorescence microscopy. A particular advantage of the antibody combination provided herein, is that this antibody combination does not only detect HPV-16 E7 but also other E7 of further high risk HPVs and in particular HPV-31 E7. Accordingly, the combination of antibodies as provided herein is a tool for detecting besides HPV-16 and HPV-18 also other HPVs, in particular HPV-31, HPV-35, HPV-45 and HPV-59. Other detections are demonstrated in the appended examples.

The invention also provides for the use of a combination of antibodies of the invention for the preparation of a diagnostic composition for the (immuno-) histological detection of E7 protein of high risk HPV in a biological sample. In accordance with the present invention by the term “biological sample” is intended any biological sample obtained from an individual, cell line, tissue culture, or other source containing polynucleotides or polypeptides or portions thereof. As indicated, biological samples include body fluids (such as blood, sera, plasma, urine, synovial fluid and spinal fluid) and tissue sources found to express the polynucleotides of the present invention. Methods for obtaining tissue biopsies and body fluids from mammals are well known in the art.

Preferably, said (immuno-) histological detection is carried out on Pap-smears (cervical smears), cervical (carcinoma) biopsies or prostate biopsies, like fine needle aspiration biopsies. It is also envisaged that said (immuno)histological detection is carried out on smears and/or biopsies of anogenital dysplasias. Such dysplasias may lead to, inter alia, anal squamous intraepithelial lesions and neoplasias (ASIL, AIN) or anal, penile and reproductive tract cancers. In this context, high risk HPV diagnostic, in particular diagnosis of the herein described high risk HPV, more preferably, HPV-16, HPV-18, HPV-31 and HPV-45 E7 protein is envisaged which comprises the analysis of samples derived from men, belonging to risk groups of sexually transmittable diseases, like bisexual and homosexual men. Yet, the diagnostic compositions described herein are useful in diagnostic settings of both, men and women, and independently from their sexual orientation. Also envisaged is the use of the inventive combination of antibodies and the diagnostic composition described herein in the detection of expressed E7 protein of high risk HPV in smears and biopsies of head and neck tissue, mamma tissue, prostate tissues, penile tissue, cervix tissue and the like. It is envisaged that the combination of antibodies of the present invention is used for the (immuno-) histological detection of E7 protein from high risk HPV such as HPV 16, HPV-18, HPV-31, HPV-33, HPV-35, HPV-39, HPV-45, HPV-52, HPV-56, HPV-58 and/or HPV-59. Most preferably, said combination of antibodies is used for detection of HPV-16, HPV-18, HPV-31, HPV-45 and/or HPV-59. It is understood that also combinations of HPV-16,18, HPV-31, HPV-45 and HPV-59 are detected by said combination of antibodies.

Most preferably said diagnostic composition is used for evaluating the acquisition of a sexually transmitted disease or the risk of developing cancer, for measuring the status of an existing sexually transmitted disease or cancer, or for screening the therapy efficiency in the treatment of a sexually transmitted disease or cancer.

Furthermore, the invention relates to a method for the preparation of a diagnostic composition comprising the step of formulating the inventive combination of antibodies with a diagnostically acceptable carrier, diluent, buffer, or storage solution. It is also envisaged that in the use or the method of the present invention, said diagnostic composition further comprises suitable means for detection, for example secondary labeled antibodies or fragments thereof.

A variety of techniques are available for labeling biomolecules, are well known to the person skilled in the art and are considered to be within the scope of the present invention. Such techniques are, e.g., described in Tijssen, “Practice and theory of enzyme immuno assays”, Burden, RH and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds. Preferred are labels to be detected in immunohistochemical techniques.

Commonly used labels comprise, inter alia, fluorochromes (like fluorescein, rhodamine, Texas Red, Cy3, Cy5, etc.), enzymes (like, peroxidase, horse radish peroxidase, β-galactosidase, alkaline phosphatase, acetylcholinesterase), radioactive isotopes (like ³²P or ¹²⁵I), biotin, digoxygenin, colloidal metals, chemi- or bioluminescent compounds (like dioxetanes, luminol or acridiniums). Labeling procedures, like covalent coupling of enzymes or biotinyl groups, iodinations, phosphorylations, biotinylations, etc. are well known in the art. It is of note that the antibodies of the invention may also be detected by secondary methods, like indirect immuno-fluorescence. Accordingly, detectably labeled secondary antibodies may be employed in the methods and uses of the present invention.

As mentioned above, direct and indirect detection methods comprise, but are not limited to, fluorescence microscopy, direct and indirect enzymatic reactions and the detection by microscopic means as well as direct detection by eye-visible signals resulting, inter alia, from accumulation of dye-labeled antibodies or the secondary detection of antibodies. Similarly, as detailed below, the detection of E7 protein by the inventive combination of antibodies may comprise the detection of soluble or solubilized E7 protein in fluid samples or solubilized samples of high risk HPV. Such methods preferably comprise, inter alia, FACS, ELISA-, FIA-, CLIA- or RIA-tests (see also below), or the use of test sticks as described below. Commonly used detection assays comprise, accordingly, radioisotopic or non-radioisotopic methods. These comprise, inter alia, Westemblotting, overlay-assays, RIA (Radioimmuno Assay) and IRMA (Immune Radioimmunometric Assay), EIA (Enzyme Immuno Assay), ELISA (Enzyme Linked Immuno Sorbent Assay), FIA (Fluorescent Immuno Assay), CLIA (Chemioluminescent Immune Assay), lateral flow immunoassay, as well as the use of test sticks detailed herein. The combination of antibodies of the invention may also be employed to detect a potential HPV-16 and a potential HPV-18 18 infection. In this context, Western Blot analysis and the like is particularly useful, since the antibodies as described in this invention show in this method less or no cross-reactivities with other HPVs of the high risk type in said technology or in technologies where denaturated and/or non-folded proteins are to be detected.

Accordingly, the invention also provides for a diagnostic composition comprising the combination of antibodies of the invention or obtained by the method of the invention. Most preferably, said diagnostic composition is employed in immunohistological, immunocytochemical methods on (biological) tissue, cells, and/or organs. Said diagnostic composition is most preferably used in cytological smears, embedded sections or cells and/or in immunostains of e.g. cervical lesions.

Said diagnostic composition may comprise the combination of antibodies of the present invention, in soluble formaiquid phase but it is also envisaged that said antibodies are bound to/attached to and/or linked to a solid support. Said diagnostic composition may be employed in samples derived from solid tissue as well as in samples which comprise fluid probes. These fluid samples may be selected, inter alia, from blood, serum, plasma, sputum, urine, ejaculate, sperm. It is also envisaged and described herein that solid samples/probes are solubilized and tested with the diagnostic composition of the present invention. Yet, in a most preferred embodiment, the combination of antibodies/sera of the present invention (and therefore the diagnostic composition) is used on smears, like Pap-smears.

Solid supports may be used in combination with the diagnostic composition as defined herein or the antibodies, antibody fragments or antibody derivatives of the combination of antibodies of the present invention may be directly bound to said solid supports. Such supports are well known in the art and comprise, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The antibodies of the combination of antibodies of the present invention may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Appropriate labels and methods for labeling have been identified above. In a preferred embodiment said diagnostic composition comprises the use of immobilized inventive antibodies.

In accordance with this invention, cost-efficient, rapid and reliable diagnostic tests and test kits may be developed. For example, a teststick may be produced that is capable to indicate HPV induced tumor development in cell lysates of cervical smears. Such lysates are often taken from material from cervix uteri, which are routinely lysed in sample buffers. Yet, the test kits of the invention may also be employed in tests for E7 proteins of high risk HPV in other samples, like (blood) serum or lysates from further biopsies or smears, like anogenital biopsies or smears. Such a test, comprising the use of test sticks or other solid matrices, is established on the principle of a ‘lateral flow system’. It is within the skill of a person skilled in the art to develop tests/test kits or means for testing which comprise, inter alia, the preparation of a test stick directly or indirectly conjugated with the antibodies of the invention. One, non-limiting example may be the preparation a “cassette housing” with windows for sample application and optical evaluation of results (comprising test and control lines, respectively) whereby said “housing” comprises a support backing as a carrier for an analytical membrane, a sample application pad, a conjugate release pad and an absorbent pad. The conjugate release pad may be prepared with substrates, comprising (conjugated) anti-E7 antibodies of the combination of antibodies of this invention, whereby said conjugation may, inter alia, be gold- or latex conjugation. The analytical membrane area in the test window may, inter alia, be prepared with different reagents in separated lines fixed to said membrane. It is envisaged that the testline carries the inventive combination of anti-E7 antibodies and the control lines may comprise E7 protein as well as (an) secondary antibody/antibodies, like (an) anti-rabbit (or anti-goat or the like) antibody or antibodies. Furthermore, a/the control line may comprise other detections means for further/other sample compounds. In the test illustrated here, the function of the control lines is to monitor the efficiency of the test/teststick and the conjugated antibodies and to exclude false positive and negative results by interfering substances. Similar assays and test means are known in the art and comprise, inter alia, pregnancy tests based on specific antibody-antigen interactions. The test stick described herein may not only be employed in cell lysates of tissue(s) to be tested but also in body fluids, like blood, serum, plasma, sputum, urine, ejaculate, sperm and the like.

In a further embodiment the invention provides for a kit comprising a combination of antibodies comprising anti-HPV-16 and anti-HPV-18 E7 antibodies of the invention or a diagnostic composition of the invention.

Advantageously, the kit of the present invention further comprises, optionally (a) buffer(s), storage solutions and/or remaining reagents or materials required for the conduct of medical, scientific or diagnostic assays and purposes. Furthermore, parts of the kit of the invention can be packaged individually in vials or bottles or in combination in containers or multicontainer units. The kit may also comprise an instruction sheet to carry out the (diagnostic) methods of the present invention.

The kit of the present invention may be advantageously used, inter alia, for carrying out the (diagnostic) methods of the invention and could be employed in a variety of applications referred herein, e.g., as diagnostic kits, as research tools or medical tools. Additionally, the kit of the invention may contain means for detection suitable for scientific, medical and/or diagnostic purposes, like e.g. secondary antibodies as described above. The manufacture of the kits follows preferably standard procedures which are known to the person skilled in the art.

In another aspect the present invention relates to an in vitro method for the detection of high risk HPV E7 protein comprising the steps of
(a) incubating a biological sample with the combination of antibodies of the invention; and
(b) measuring and/or detecting E7 protein of high risk HPV
   whereby the presence, the absence or the amount of specifically-bound antibodies of the combination of antibodies is indicative for the presence of high risk HPV E7 protein. It is further preferred that this in vitro method comprises a further step (c), whereby in said step (c) the presence, the absence or the amount of specifically-bound antibodies of the combination of antibodies of the present invention of step (b) is compared to the presence, the absence or the amount of specifically-bound antibodies of the combination of antibodies in a negative or a positive control sample or in both control samples. The aforementioned in vitro method detects, for example, high risk HPV induced ongoing tumor diseases. Samples have been described supra. When applying the combination of antibodies of the present invention in the above-described method, it is to be understood that the term “combination” is not envisaged to be strictly construed in the sense that the antibodies of the combination of antibodies are to be applied together, i.e. in combination which each other. Accordingly, the anti-HPV-16 E7 antibody which is comprised in the combination of antibodies may be applied at first and, subsequently, the anti-HPV-18 E7 antibody which is comprised in said combination of antibodies or vice versa. Thus, the order of applying the antibodies of the combination of antibodies is irrelevant. However, it is also envisaged that the anti-HPV-16 E7 and the anti-HPV-18 E7 antibody may be applied together. In order to detect with the inventive combination of antibodies a potential infection with HPV-16, HPV-18 or other high risk HPVs immunocytochemical and/or immunohistological methods on cells, tissues or organs are preferably carried out.

The measurement and/or detection of specifically “bound antibodies of the combination of antibodies” may be carried out as described above, for example by the detection of directly or indirectly labeled, bound antibody molecules of the invention. Said measuring and detection methods may also comprise automated and/or computer-controlled detection methods.

Such in vitro methods of the invention are described herein and are also illustrated in the appended Examples and may be, inter alia, employed to detect the presence or absence of an infection with a high risk HPV, to evaluate whether a high risk HPV infection is merely transient or an asymptomatic HPV infection. The combination of antibodies of the present invention enables the differentiation between high risk HPV and low risk HPV as described supra. It is of note that the combination of antibodies of the present invention may be employed in the above described method in order to evaluate the absence or presence of a proliferative disorder, like, e.g. cervix carcinoma, prostata carcinoma, breast cancer, anogenital cancer, penile cancer and head and neck cancer. Furthermore, the combination of antibodies may be employed to evaluate the class of a proliferative disorder, for example it can be evaluated whether a prostatic carcinoma is high risk HPV dependent or independent. It is, e.g., envisaged that patients whose serum comprises prostatic-specific antigen (PSA) or who have a positive result in fine-needle aspiration biopsies of prostatic tissue are further examined for the presence or absence of high risk HPV, employing the combination of antibodies of the invention and methods disclosed herein. Such a diagnostic method allows for the distinction of high risk HPV E7-positive and high risk HPV E7-negative prostate carcinomas and the medical intervention may be chosen accordingly.

As mentioned above, the biological sample is preferably a cervix or a prostatic sample, most preferably a Pap-smear or a fine needle aspiration biopsy.

In contrast to previous technology, namely the detection of high risk HPV, e.g. HPV-16 DNA in prostate cancer biopsies, the detection of high-level E7 expression of high risk HPV in prostate cancer samples allows the conclusion that in these samples the E7 oncoprotein, which is the major transforming protein of the virus, is actively expressed. The person skilled in the art knows that expression of the E7 oncoprotein in any cell results in the inactivation of several important tumor suppressor mechanisms, as reviewed in Zwerschke (2000, Adv. Cancer Res. 78, 1-29). This indicates that there is a high risk for malignant progression of this lesion. It has to be stressed that currently physicians do not consider HPV-dependent malignant progression of prostate cancers, since detection of HPV oncoproteins in prostate cancer specimens was not possible with the techniques of the prior art. The combination of antibodies described herein allow significant progress in clinical research since it is now possible to detect E7 protein of high risk HPV which are the causative agent of the diseases disclosed herein. Furthermore, it is anticipated that with the advent of specific antiviral drugs and/or treatments directed against high- risk papillomaviruses, the detection of high risk HPV, for example HPV-16, HPV-18, HPV-31 or HPV-45 E7 in prostate cancer specimens will direct the physician to new modes of treatment for this important malignancy.

In accordance with this invention, the biological sample to be tested and/or evaluated with the inventive combination of antibodies may be a solid sample as well as a soluble/solubilized sample. Even if one of the most preferred uses of the inventive combination of antibodies comprises the diagnostic use in immunohistochemical assays, in particular on smears, further methods of diagnosis employing the inventive combination of antibodies are envisaged in this invention. These further methods are described and illustrated herein and comprise the use of solid and non-solid phase immunoassays, like ELISA-, RIA-tests or the use of (antibody-covered) tests sticks, magnetic or polystyrol beads and the like.

Besides samples derived from cervix, anogenital tissue head- and neck tissue and/or prostatic tissue, it is also envisaged that the inventive combination of antibodies is employed in diagnostic samples derived from mamma/mamma tissue. The combination of antibodies of the present invention is particularly useful in screening of mamma tissue obtained from patients who suffer or had suffered from a cervix carcinoma and may develop, e.g. due to metastasis, a mamma carcinoma. Accordingly, the present invention also relates to an in vitro method for detection of a mamma/breast cancer, in particular of a mamma cancer in a patient who suffers or who has suffered from, in particular a cervix carcinoma/cervical cancer. Said in vitro method comprises the incubation of mamma tissue (solid or solubilized) with the combination of antibodies of the invention and the measurement and/or detection of specifically bound antibodies of the combination of antibodies, whereby the presence, the absence or the amount of specifically bound antibodies of the combination of antibodies is indicative for mamma/breast cancer. In particular, a positive signal of specifically bound E7 antibodies of the combination of antibodies of the present invention is indicative for a mamma carcinoma/breast cancer, in particular a mamma carcinoma being a secondary tumor or a metastasis from a primary tumor, like a cervix carcinoma or an anogenital cancer.

In a preferred embodiment of the present invention the above-described method is used to detect E7 protein of high risk HPV, e.g. the potential infection with a high risk HPV as defined herein and as known in the art. Particularly preferred are HPV-16, HPV-18, HPV-31 and /or HPV-45. It is to be understood that said method may be used to detect any of HPV-16, HPV-18, HPV-31 or HPV-45 alone or in any possible combinations.

In another preferred embodiment, the above-described method is used for determining the occurrence of a sexually transmittable disease or cancer which may be caused by the herein disclosed high risk HPV.

The invention, accordingly, provides for the use of a combination of antibodies, a diagnostic composition or a kit of the invention in an in vitro method for the detection of E7 protein of high risk HPV. Said sexually transmitted disease is, preferably a high risk HPV-infection or said cancer is cervical cancer, breast cancer, prostate cancer, anogenital cancer/anogenital neoplasia (AIN), penile cancer or head and neck cancer as described herein. The feasibility of a successful HPV-diagnostic, in particular high risk HPV diagnostic on smears is described in the appended Examples.

In another embodiment, the present invention provides for a method for production of a combination of an anti-HPV-16 E7 antibody and an anti-HPV-18 E7 antibody comprising the steps of
(a) eliciting an in vivo humoral response against highly purified, HPV-16 E7 protein or a fragment thereof in a goat;
(b) affinity-purifying antibodies as obtained in the eliciting-step (a); and
(c) mixing the antibody of step (b) with an anti-HPV-18 E7 antibody.

In a most preferred embodiment, the highly purified HPV-16 E7 proteins or a fragment thereof to be used in the immunization protocol described herein and illustrated in the appended Examples is a native, highly purified HPV-16 E7 protein or a fragment thereof. The term “native” as used in accordance with this invention is explained herein above and illustrated in the appended Examples. With respect to the preferred embodiments the same applies, mutatis mutandis, as described herein above for the antibodies comprised in the combination of antibodies of the present invention.

Since it was unexpectedly found and also demonstrated in the appended Examples herein below that an antibody raised against E7 protein of HPV-16 in a goat which is obtainable by steps (i) and (ii) as described herein above recognizes due to cross- reactivity not only E7 protein of HPV-16, but also E7 protein of other HPV strains, preferably HPV strains of the high risk group of HPV, the present invention relates in another aspect to the use of the anti-HPV-16 E7 antibody obtainable by steps (i) and (ii) as described above for detecting E7 protein of HPV-31, HPV-33, HPV-35, HPV-39, HPV-45, HPV-52, HPV-56, HPV-58, HPV-59, HPV-68, HPV-69, HPV-73 and/or HPV-82 or any possible combination thereof, preferably HPV-31, 33, 35, 39, 45, 52 58 and/or 59 or any possible combination thereof. Said antibody may not recognize E7 protein of HPV-1, 6 and/or 11, preferably it does not recognize E7 protein of HPV-11.

Another embodiment of the present invention relates to a diagnostic composition comprising the antibody obtainable by steps (i) and (ii) as described above.

It is furthermore envisaged that the anti-HPV-16 E7 antibody obtainable by steps (i) and (ii) as described above may be used for the preparation of a diagnostic composition for detecting E7 protein of HPV-31, HPV-33, HPV-35, HPV-39, HPV-45, HPV-52, HPV-56, HPV-58, HPV-59, HPV-68, HPV-69, HPV-73 and/or HPV-82 or any possible combination thereof. Said anti-HPV-16 E7 antibody obtainable by step (i) and (ii) as described above is particularly useful for the detection of E7 protein of HPV-31, HPV-33, HPV-35, HPV-39, HPV-45, HPV-52, HPV-56, HPV-58 and/or HPV-59 or any possible combination thereof. However, the anti-HPV-16 E7 antibody obtainable by step (i) and (ii) as described above may not recognize E7 protein of HPV-1, HPV-6 and/or HPV-11. Preferably, said antibody does not recognize E7 protein of HPV-11. The detection is made by techniques commonly known in the art and described hereinabove, preferably in a biological sample as described herein above.

A method of preparation of the aforementioned diagnostic composition comprising the step of formulating the antibody obtainable by steps (i) and (ii) as described above with a diagnostically acceptable carrier, diluent, buffer or storage solution is also envisaged by the present invention.

Moreover, in another embodiment the present invention encompasses a kit comprising the anti-HPV-16 E7 antibody obtainable by step (i) and (ii) as described above or the aforementioned diagnostic composition.

The present invention also relates to an in vitro method for the detection of E7 protein of HPV-31, 33, 35, 39, 45, 52, 56, 58 and/or 59 or any possible combination thereof comprising the steps of
(a) incubating a biological sample with the anti-HPV-16 E7 antibody obtainable by step (i) and (ii) as described above; and
(b) measuring and/or detecting E7 protein of HPV-31, 33, 35, 39, 45, 52, 56, 58 and/or 59 or any possible combination thereof,
   whereby the presence, the absence or the amount of specifically-bound antibodies is indicative for the presence of HPV-31, 33, 35, 39, 45, 52, 56, 58 and/or 59 E7 protein or any possible combination thereof.

With respect to the preferred embodiments relating to the anti-HPV-16 E7 antibody obtainable by steps (i) and (ii) as described above the same preferred embodiments apply, mutatis mutandis, as described herein above for the combination of antibodies of the present invention.

The Figures show:

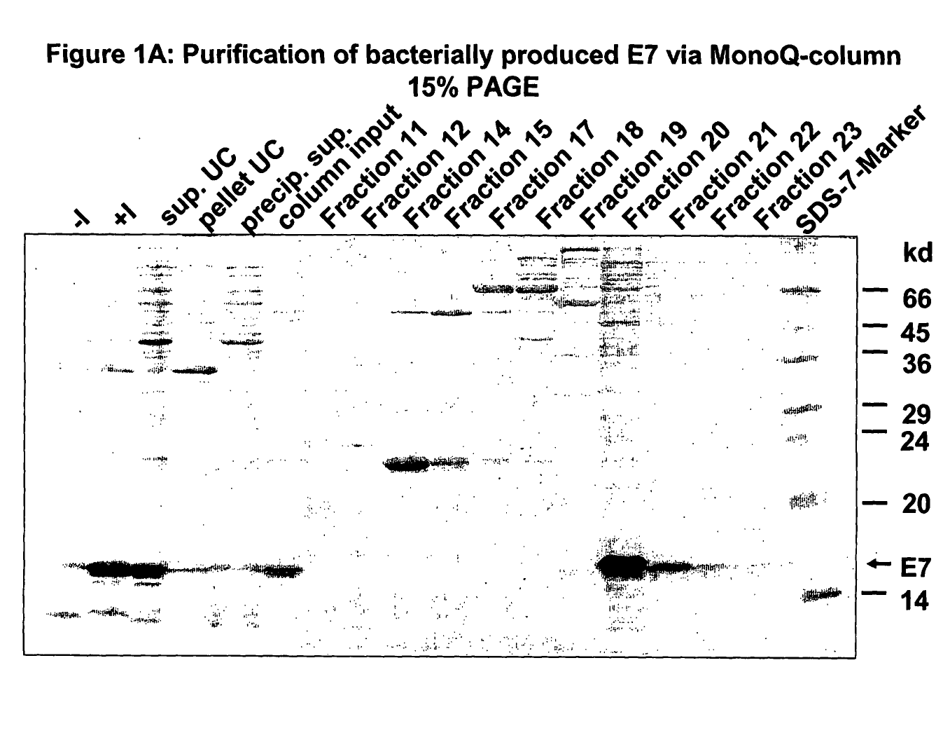

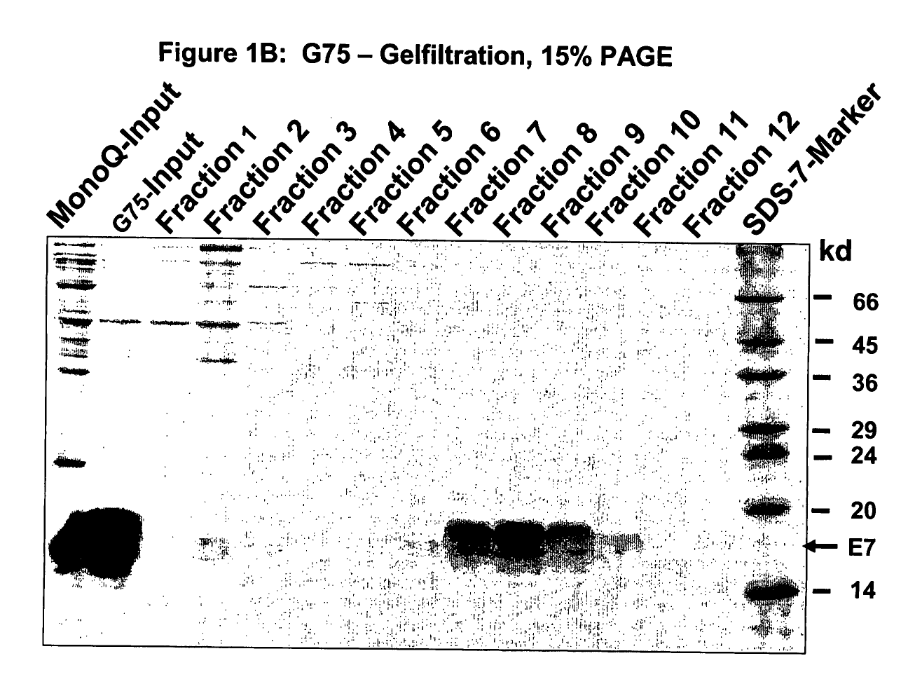

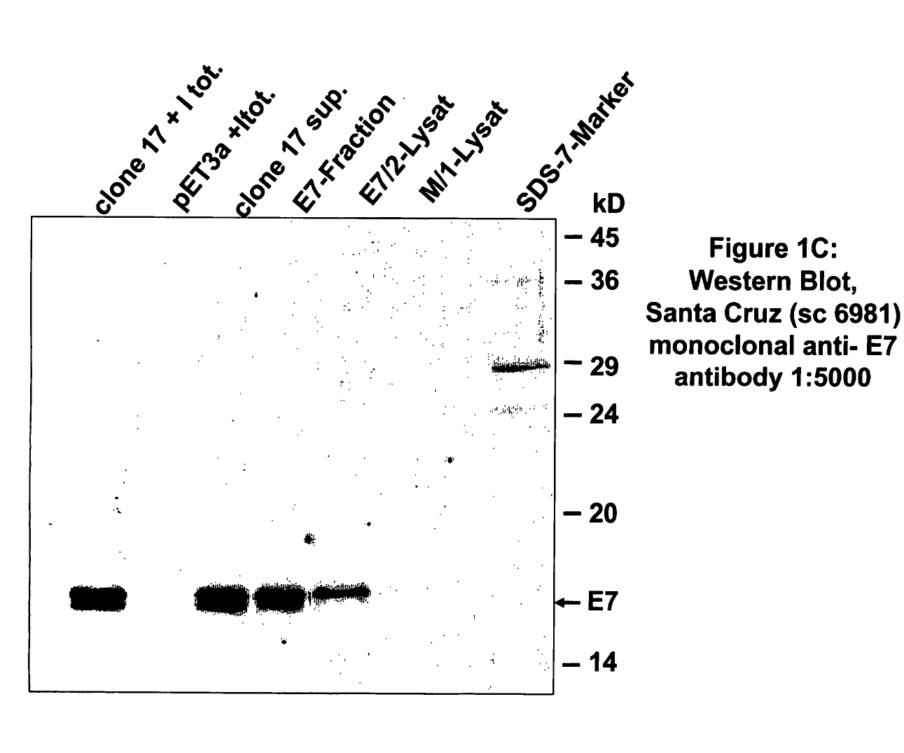

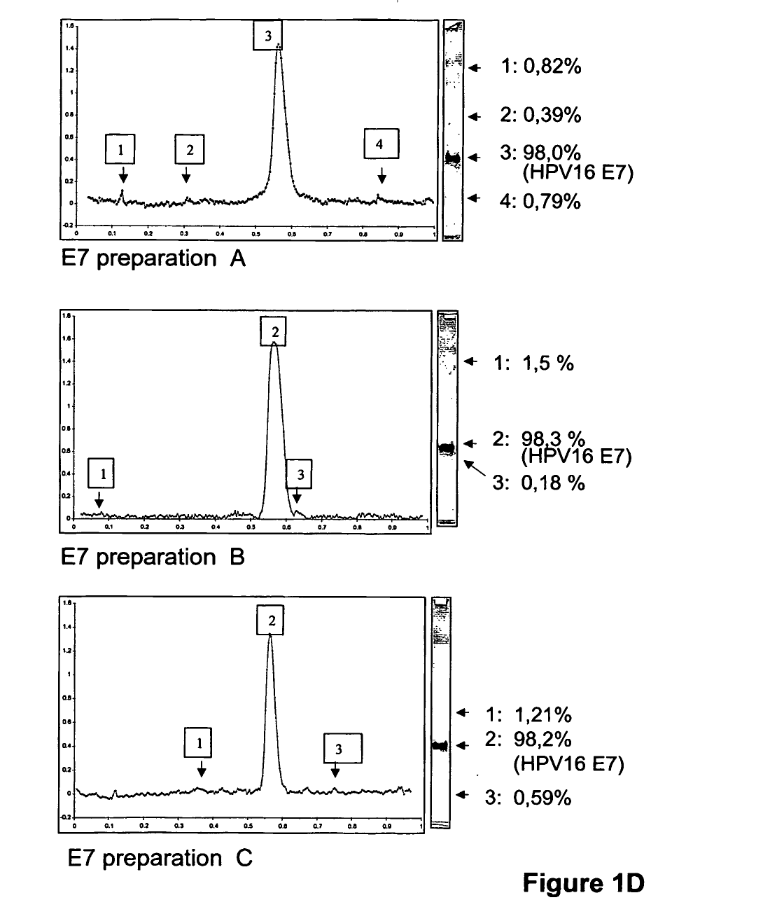

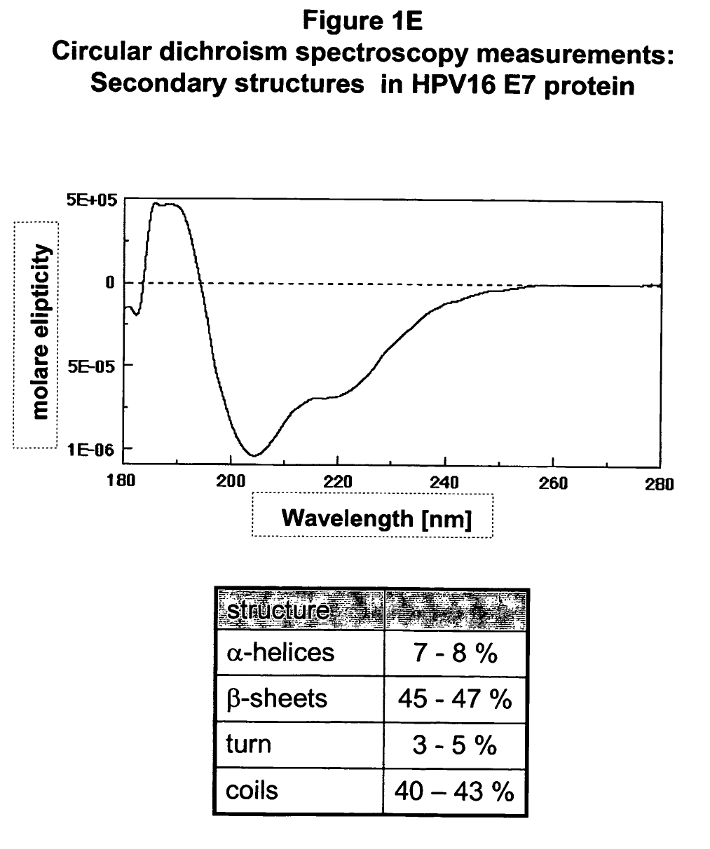

FIG. 1: Purification of the HPV-16 E7 oncoprotein. Bacterial expressed recombinant HPV-16 E7 was stepwise purified by ammonium sulfate precipitation, anion-exchange chromatographie on MonoQ and Gelfiltration on a Sephadex G75 column. (1A, 1B) Samples were separated by gel electrophoresis, and purification was documented by coomassie staining of the fractions as indicated. Identity of the isolated protein and purity of the HPV-16 E7 protein was confirmed by Western blotting using a monoclonal anti E7 antibody (Santa Cruz, Vienna, Austria) (1C). Three different preparations of recombinantly expressed HPV 16 E7 protein were evaluated to detect, the purity of the preparations and the reproducibility of the applied methods. The amount of proteins separated per lane was 0.1 μg HPV16-E7 protein. The gel was silver stained according to Heukeshoven and Demick (in R. Westermeier et al. 1990; ISBN 3-527-28172-X) for 30 min. The gel was scanned using a Fluor-S™ Multi-Imager system (BIORAD). Cross sections of defined lanes were saved as TIFF images using Quantity One (Quantitation Software by BIORAD). Evaluation of the get-bands was performed by using TOTALLab evaluation software Version 1.1. The sum of all pixels over the entire length of one lane was assumed to be equivalent to 100% of protein applied (0.1 μg/lane). E7 concentration and purity was 98.0% (A), 98.3% (B) and 98.2% (C) (1D). A preparation of recombinantly expressed and highly purified HPV-16 E7 protein as described herein was evaluated for secondary structure elements in the native folded protein in a physiological solvent by CD spectroscopy. The native protein to be employed for immunization protocols is folded into secondary structure elements like β-sheets (45-47%), coils (40-43%), α-helices (7-8%) and turns (3-5%). Yet, also fragments of the native, highly purified E7 proteins as described herein may also be employed in immunization protocols (1 E);

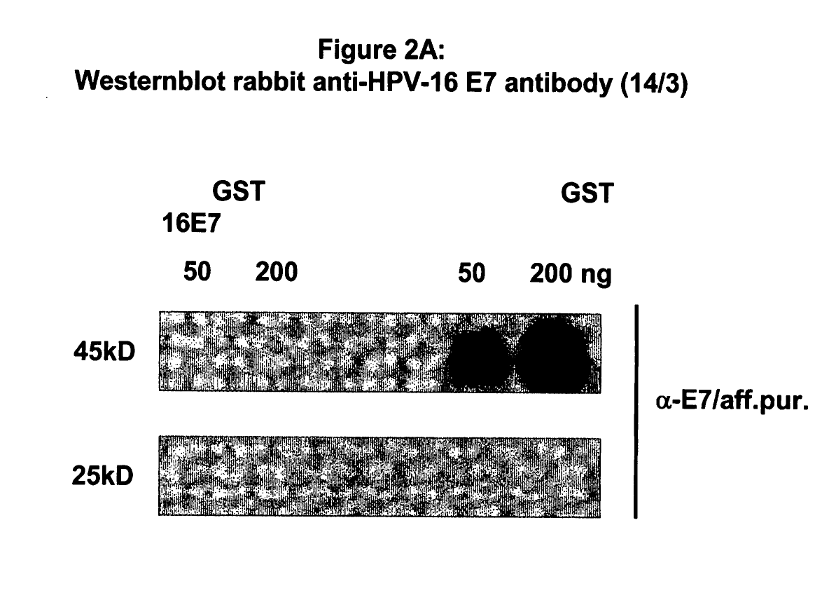

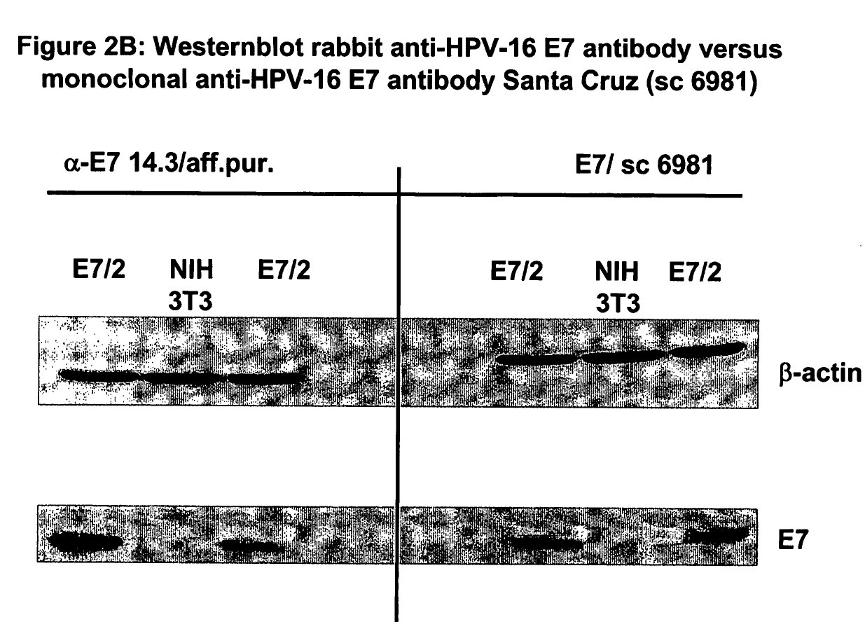

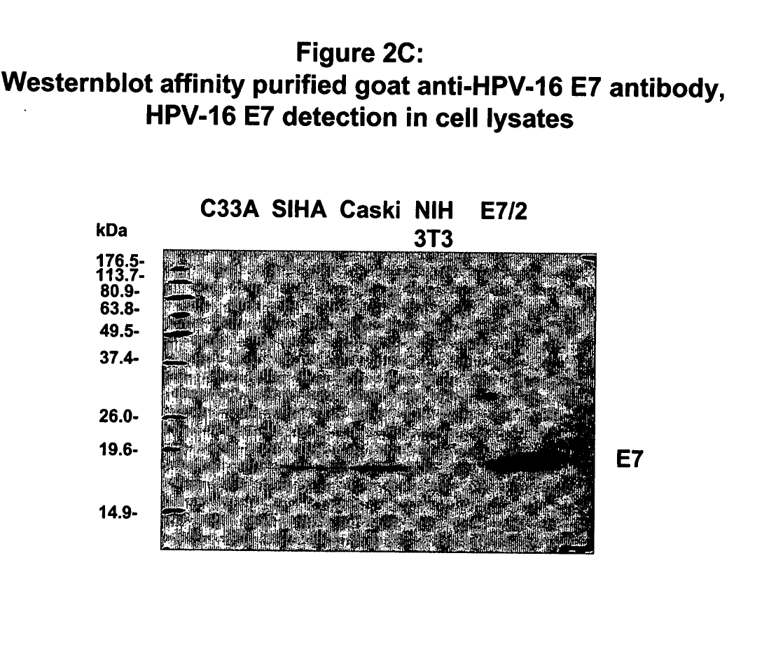

FIG. 2: Test of the affinity purified rabbit anti-HPV-16 E7 antibodies (14/3) in westernblot analysis. Purified GST and GST-HPV-16 E7 proteins were separated by SDS-polyacrylamide gel electrophoresis, and the GST-HPV-16 E7 protein was detected by westernblotting. (2A) The HPV-16 E7 expressing cells E7/2 and the control cells were subjected to lysis. Subsequently lysates were separated by SDS-polyacrylamide gel electrophoresis and probed with antibodies to HPV-16 E7 and beta actin (input control), as indicated (2B). Test of the affinity purified goat anti-HPV-16 E7 antibodies in western blot analysis. Lysates of different HPV-16 E7 expressing cell lines (SIHA, CASKI, E7/2) and the control cells NIH3T3 and C33A were separated by SDS-PAGE gel electrophoresis and probed with goat anti-HPV-16 E7 antibodies (2C).

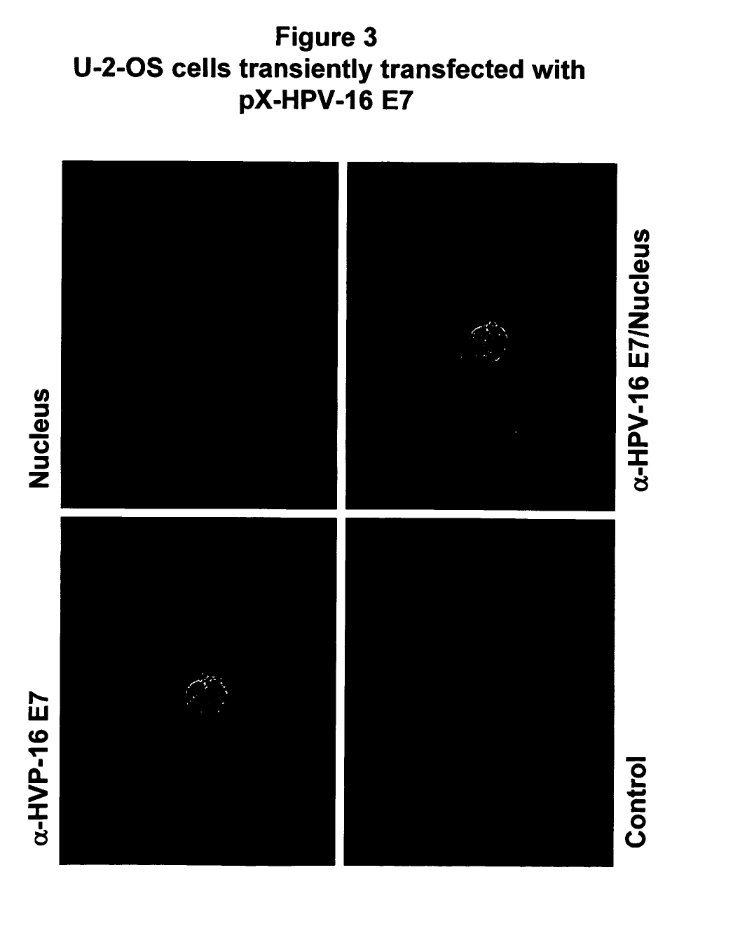

FIG. 3: Detection of HPV-16 E7 protein after transient expression in human cells. U-2OS cells were transiently transfected with expression vectors for HPV-16 E7, as indicated. At 26 h post transfection, cells were processed for indirect immunofluorescence microscopy and viewed by using a confocal scanning system. Cells were stained with rabbit anti-HPV-16 E7 antibodies clone 14/3 (anti-HPV-16 E7), preimmune serum (control), TroPro3 (nucleus) or both anti-E7 antibodies and TroPro3 (anti-HPV-16 E7/nucleus), as indicated.

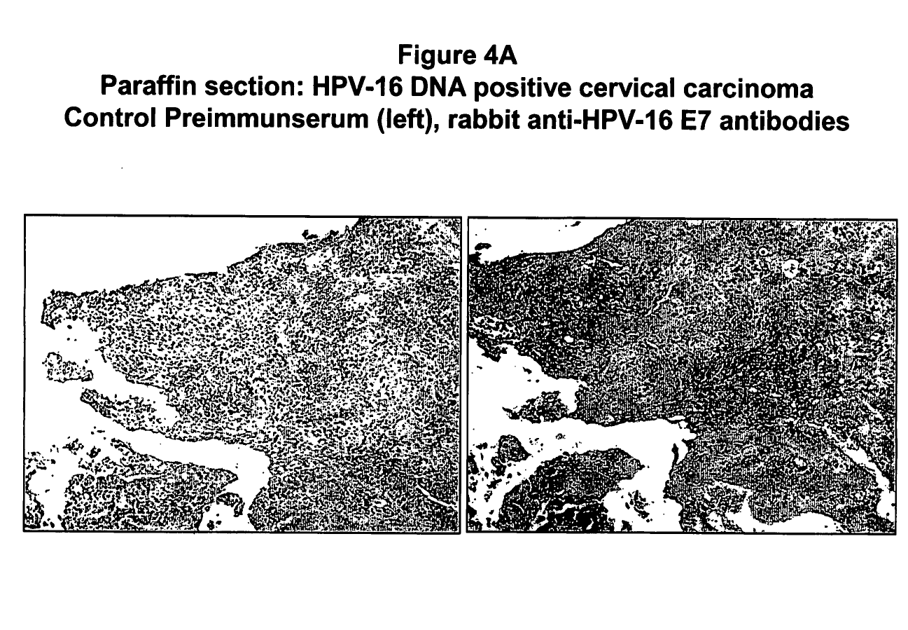

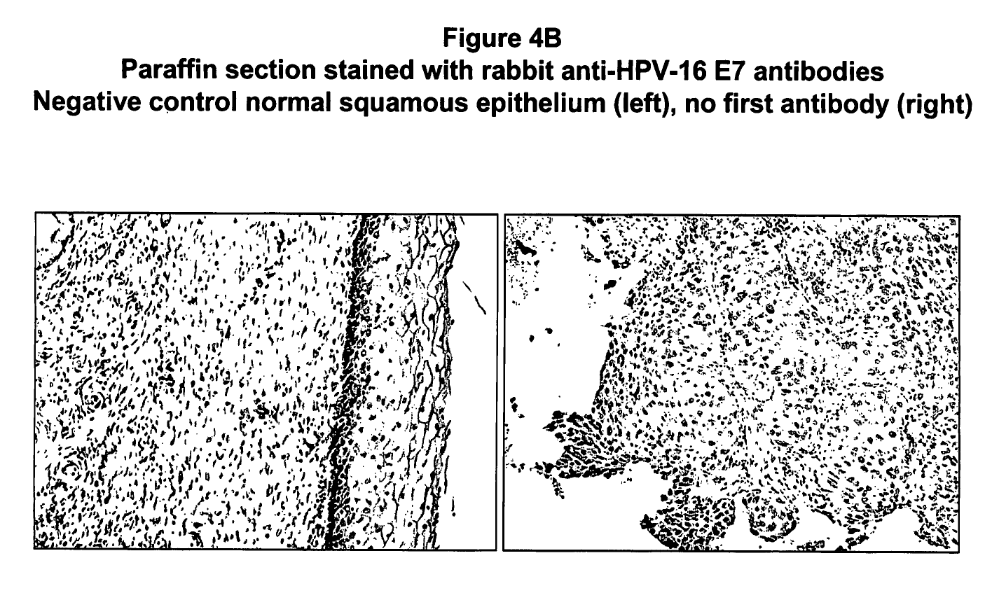

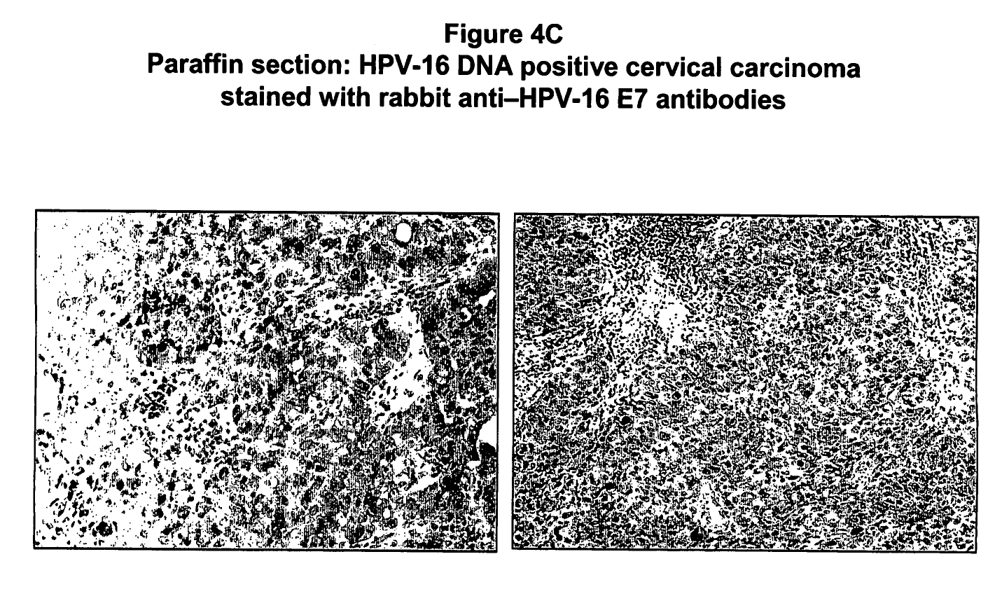

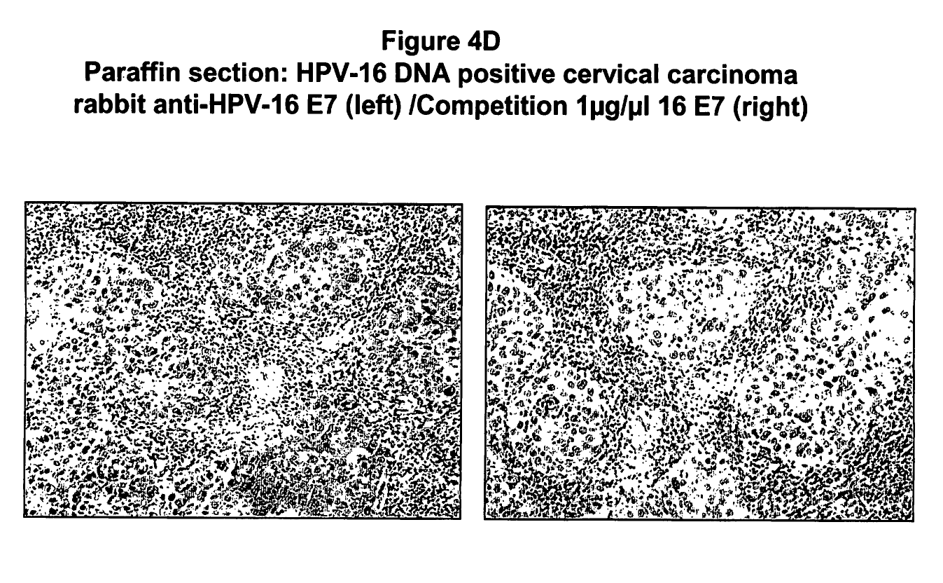

FIG. 4: Immunoperoxidase staining of paraffin sections of normal cervix and cervical carcinomas with affinity purified rabbit polyclonal antibodies against HPV-16 E7. Paraffin sections of normal cervix and a cervical carcinoma were immunostained for HPV-16 E7 by the immunoperoxidase method as described in Example 2, infra. In cervical carcinoma tissue, epithelial cells are negative with the preimmunserum (4A, left). In cervical carcinoma tissues rabbit anti-HPV-16 E7 antibodies stain virtually all cells in the tumor islets (4A, right; 4C; 4D, left). In normal cervical tissue, epithelial cells are negative with these antibodies (4B, left). In cervical carcinoma tissues staining by the rabbit anti-HPV-16 E7 antibodies can be competed out by preincubation of the antibodies with purified HPV-16 E7 antigen (4D, right). Control, no staining of cervical carcinoma tissues was obtained by adding only the horse radish conjugated secondary anti rabbit IgG (4B, right).

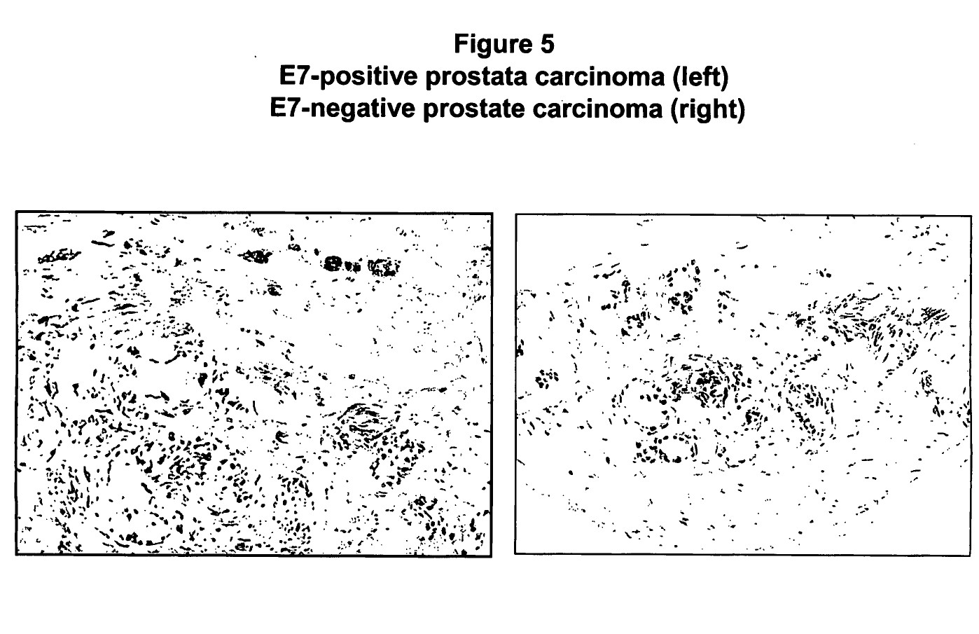

FIG. 5: Immunoperoxidase staining of cells obtained from prostate carcinoma patients. Biopsies were taken from 60 prostate carcinoma patients and samples from 60 patients were applied to an object slide together with negative controls. These slides are known to the expert as “tissue microarrays” (Skacel, 2002, Appl. Immunohistochem. Mol. Morphol. 10, 1-6). Tissue micorarrays were stained with rabbit anti-HPV-16 E7 antibodies to HPV-16 E7 as described in FIG. 4 for cervical biopsies. In this experiment, a subset of the carcinoma biopsies stained positive for HPV-16 E7, whereas other biopsies from different prostate cancer patients stained negative.

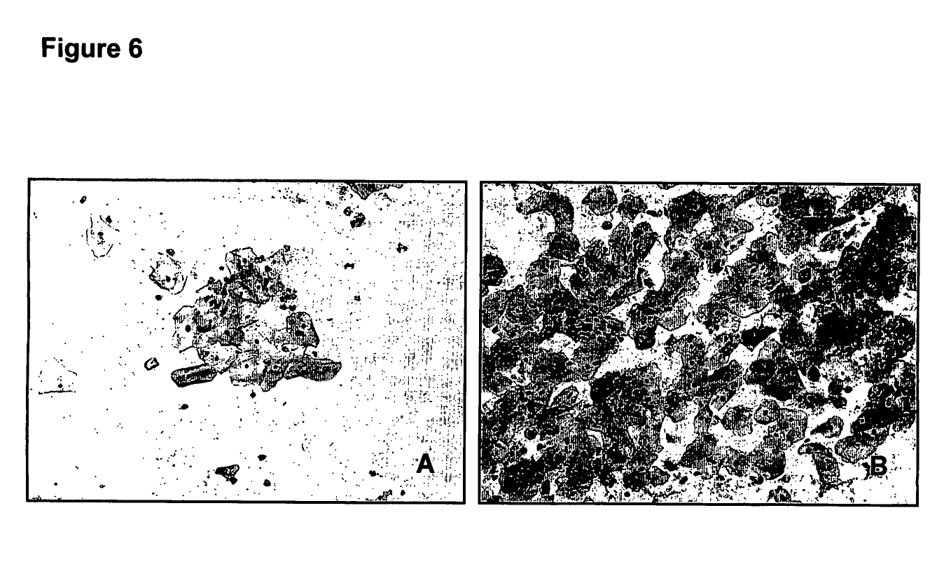

FIG. 6: Cells from surface layers of the ectocervical epithelium were spread out on glass object slides and immunoperoxidase stained by the rabbit anti-HPV-16 E7 antibodies (brown). The cells were counterstained with Hemalaun (grey/blue) and viewed by brightfield microscopy. The HPV-DNA status of the specimens was analyzed by PCR. No brown staining was observed in cells from normal (Pap II) HPV-DNA negative ectocervical smear (A). Cells from HPV-16 DNA positive cytological abnormal (Pap IIID) ectocervical smear were stained brown by the antibodies (B).

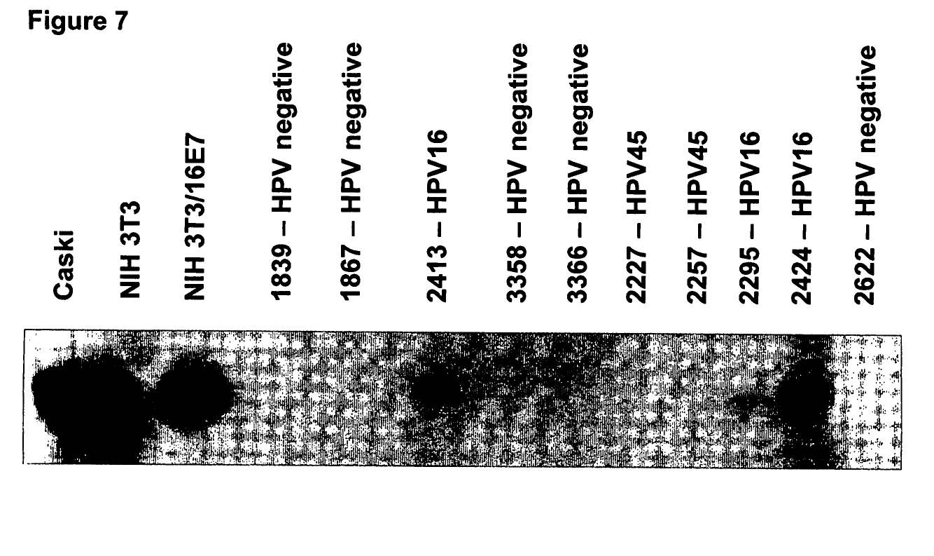

FIG. 7: Expression of the HPV-16 E7 oncoprotein in biopsies derived from cervical carcinoma patients. Three HPV-16 positive cervical carcinomas and seven HPV-16 negative cervical tissues were analysed for the expression of the HPV-16 E7 protein. Lysates, 0.5 mg each, were separated by SDS-polyacrylamide gel electrophoresis, and the HPV-16 E7 protein was detected by Western blofting. As controls, lysates from CaSki cells, an established cervical carcinoma cell line (obtained from DKFZ Heidelberg, Germany), NIH3T3 mouse fibroblasts and NIH3T3/16E7 cells, a cell line derived from NIH3T3 cells by stable transfection with the pMOHPV16E/ expression vector (Edmonds (1989), J. Virol. 63, 2650-2656), were assayed.

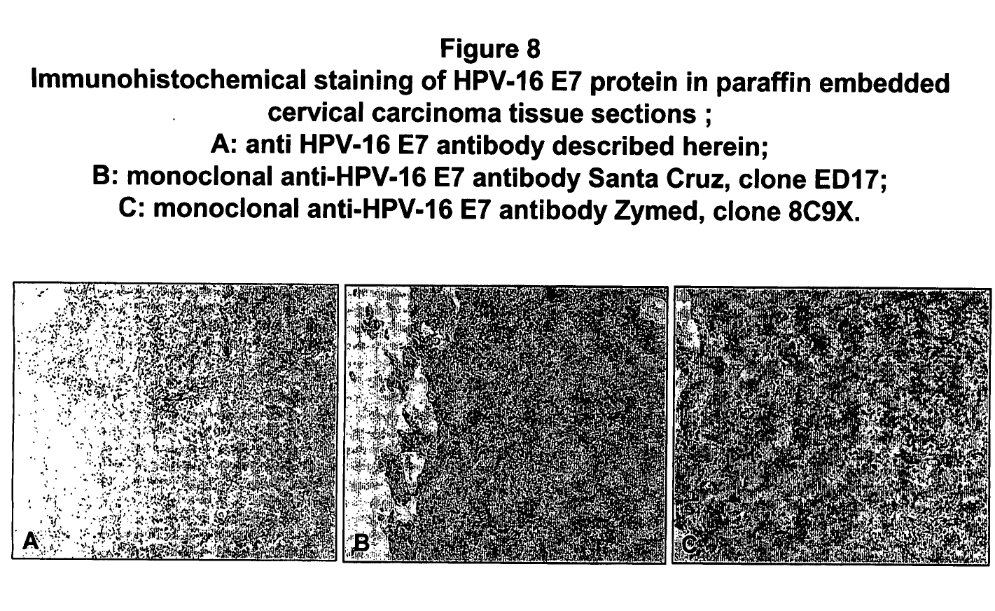

FIG. 8: Comparison of immunohistochemical staining of HPV-16 E7 protein in paraffin embedded cervical carcinoma tissue sections (consecutive sections from one tissue slice) by two different monoclonal anti HPV-16 E7 antibodies (Santa Cruz, Zymed) and the rabbit polyclonal anti HPV-16 E7 antibody described herein. Immunohistochemical staining was performed as described in Example 8. Antibodies was diluted according to manufacturers protocol. In cervical carcinoma tissues rabbit anti-HPV-16 E7 antibodies described herein stain virtually all cells in the tumor islets (A). No clear signal was obtainable in cervical carcinoma tissues by the monoclonal anti HPV-16 E7 antibodies ED17 (Santa Cruz) and 8C9X (Zymed) (B,C). In the latter cases, a high and apparently unspecific background is not restricted to the area that is cytologically recognized as tumor tissue, but were also present in the non-tumor tissue.

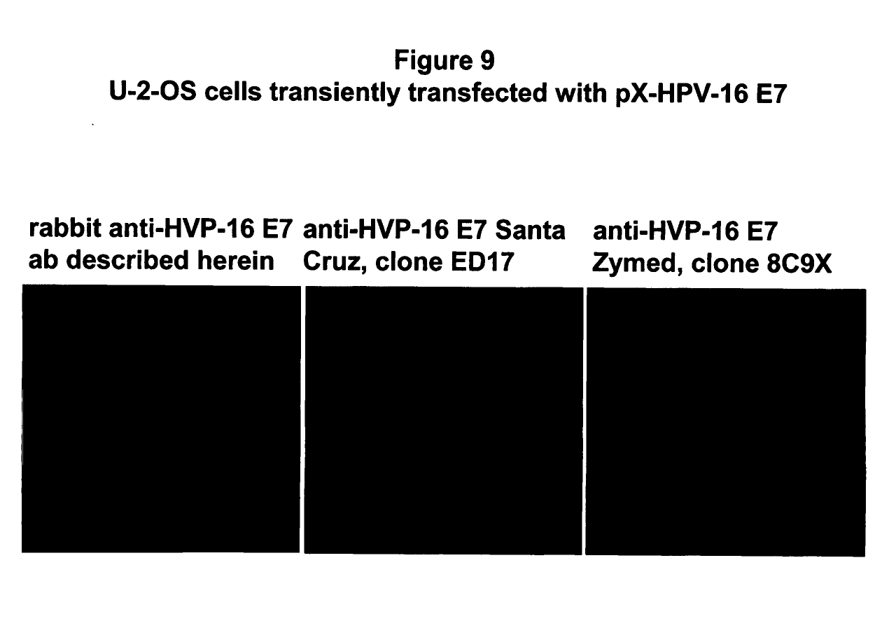

FIG. 9: Comparison of indirect immunofluorescence detection of HPV-16 E7 protein in transiently transfected U-2OS cells by the rabbit polyclonal anti HPV-16 E7 antibodies described herein and two commercialized monoclonal anti HPV-16 E7 antibodies (Santa Cruz, Zymed). The staining was performed as indicated in FIG. 3 and Example 3.5, infra.

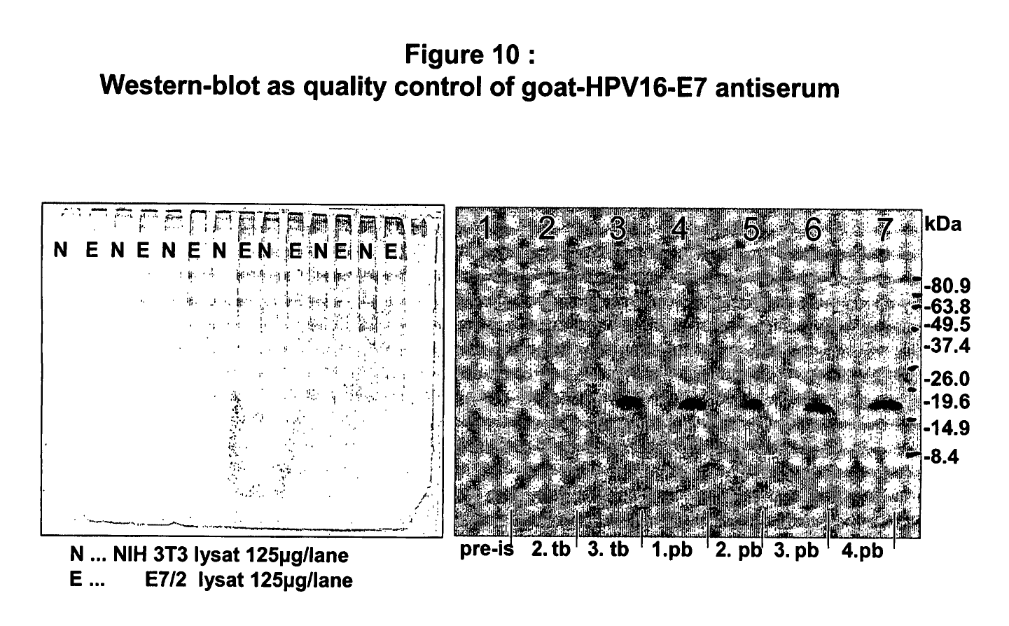

FIG. 10 shows a Western blot to investigate immune-response and quality of generated goat anti-sera. After the transfer of protein to PVDFmembrane, membrane was cut into 7 stripes. Each of the 7 stripes contained 125 μg of NIH-3T3 cell lysate (left lane) and 125 μg of E7/2 lysate (right lane). Incubation of the PVDF-stripes was as follows: stripe 1—pre-immune-serum; stripe 2—2^{nd} test bleeding; stripe 3—3^{rd} test bleeding; stripe 4—1^{st} production bleeding; stripe 5—2^{nd} production bleeding; stripe 6—3^{rd} production bleeding; stripe 7—4^{th} production bleeding.

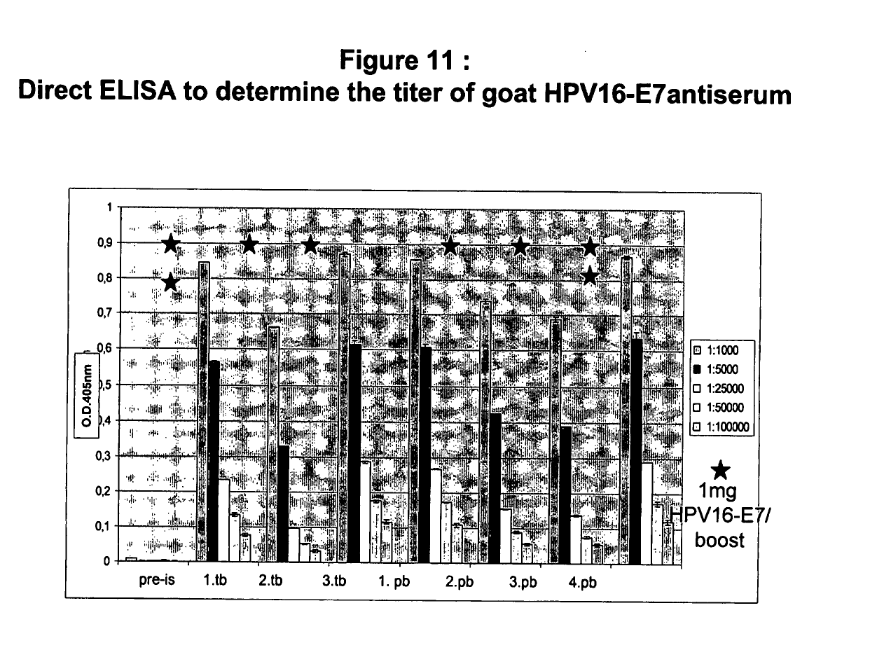

FIG. 11 Serum samples from test bleedings (tb) and production bleedings (pb) obtained from one of two goats, immunised with HPV16-E7 according to the time schedule given in Example 4.1 were used. The panel show the calculated result of the titer-ELISA after 30 min of colour development. Signals from 96-well plate lane ½ were subtracted (background control) all samples were measured in duplicates, S.D. is shown. The asterisks indicate the number of anti-gene administrations between the bleedings. In all bleedings tested, the observed titer was above 100000.

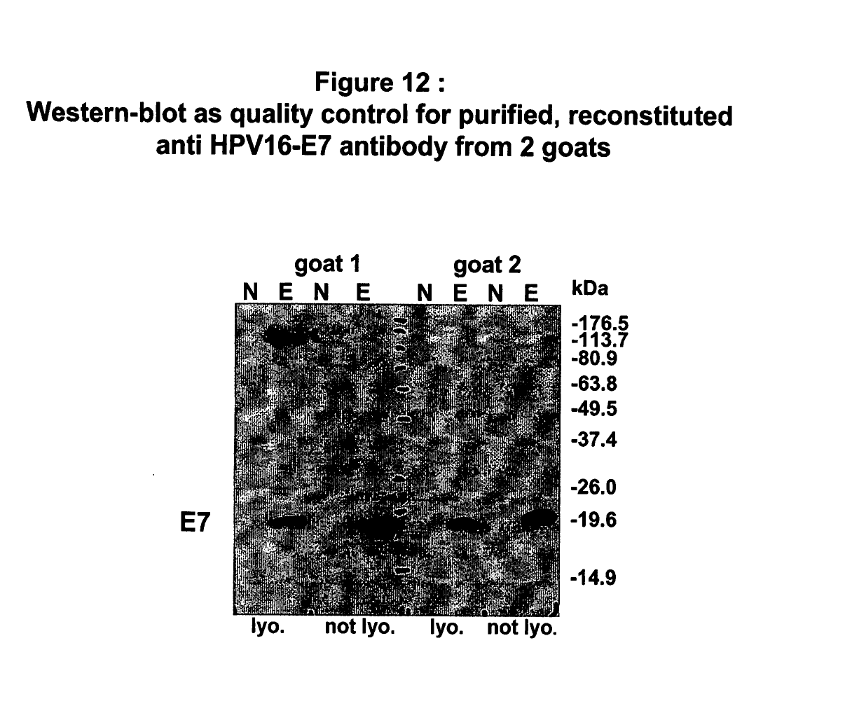

FIG. 12 shows a Western-blots as quality control for purified, reconstituted anti HPV16-E7 antibody from two goats as described in Example 4.5. Membranes were cut into stripes for antibody exposure, in the way that each stripe contained control and test lanes. Strips containing cell-lysat from NIH-3T3 fibroblast (control) and E7/2 cells (test) were probed with lyophilised (1) and not-lyophilised (2) antibodies from goat 1 and lyophilised (3) and not-lyophilised (4) antibodies from goat 2. No difference in signal-strength or loss of specificity before and after lyophilisation was observed.

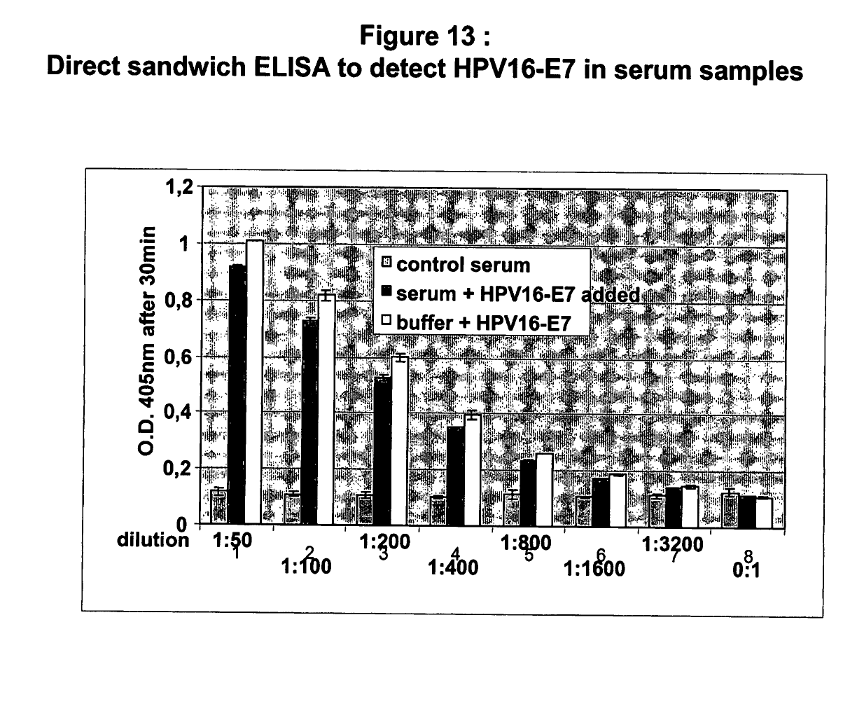

FIG. 13 The ELISA evaluation was obtained after 30 min at 37° C. as described in Example 4.7. In the control serum of the healthy donor, no specific HPV16-E7 signal was detected (even after prolonged developing time of 1.5 hours). Signals observed were regarded as back-ground. In serum and buffer containing HPV16-E7 protein respectively, a specific signal was observed down to a dilution of 1:1600-1:3200, corresponding to an antigen concentration of 62.5pg -31.25 pg/well. As expected, in the “test-serum” serum-components interfered with the added HPV16-E7 protein, thereby slightly suppressing the detection signals.

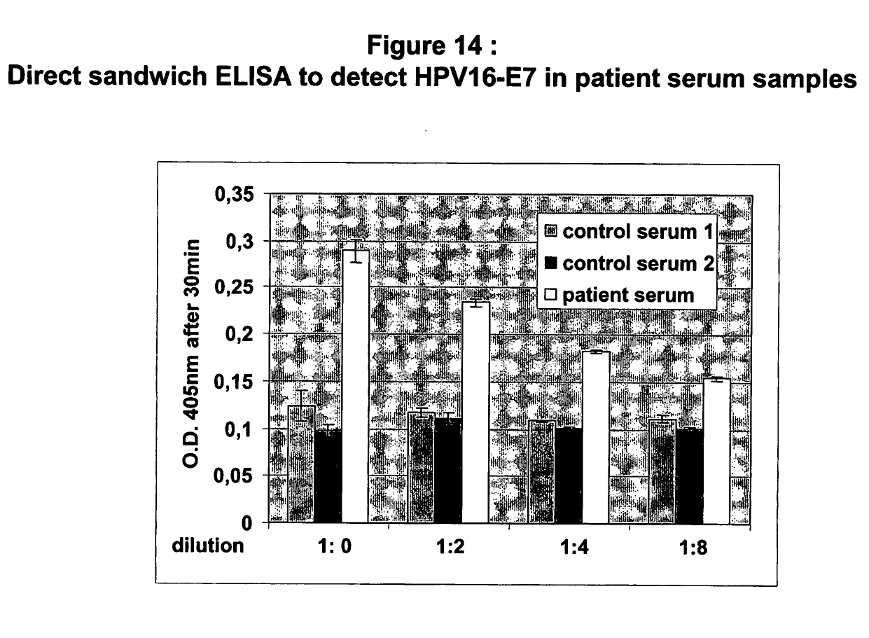

FIG. 14 The ELISA evaluation of the signals was obtained after 30 min at 37° C. as described in Example 4.8. In the two control sera of the healthy donors, no specific HPV16-E7 signal was detected. Signals observed were regarded as back-ground (see also back-ground signal from control-serum in the upper panel). In the patient serum HPV16-E7 protein could be detected throughout all dilutions.

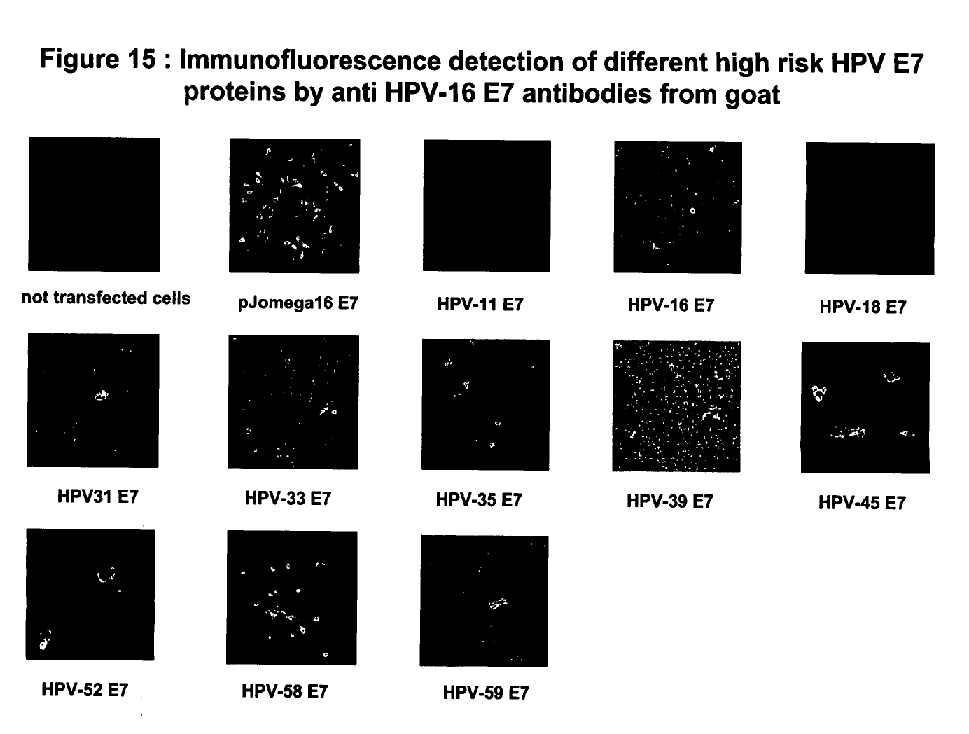

FIG. 15 Detection of different high risk HPV E7 proteins after transient expression in human cells. U-2OS cells were transiently transfected with expression vectors for HPV-11, 16, 18, 31, 33, 35, 39, 45, 52, 58 and 59 E7 proteins, as indicated. At 26 h post transfection, cells were processed for indirect immunofluorescence microscopy and viewed by using a confocal scanning system. Cells were stained with affinity purified goat anti-HPV-16 E7 antibodies. No signals was obtained in not transfected cells, HPV11 and HPV18.

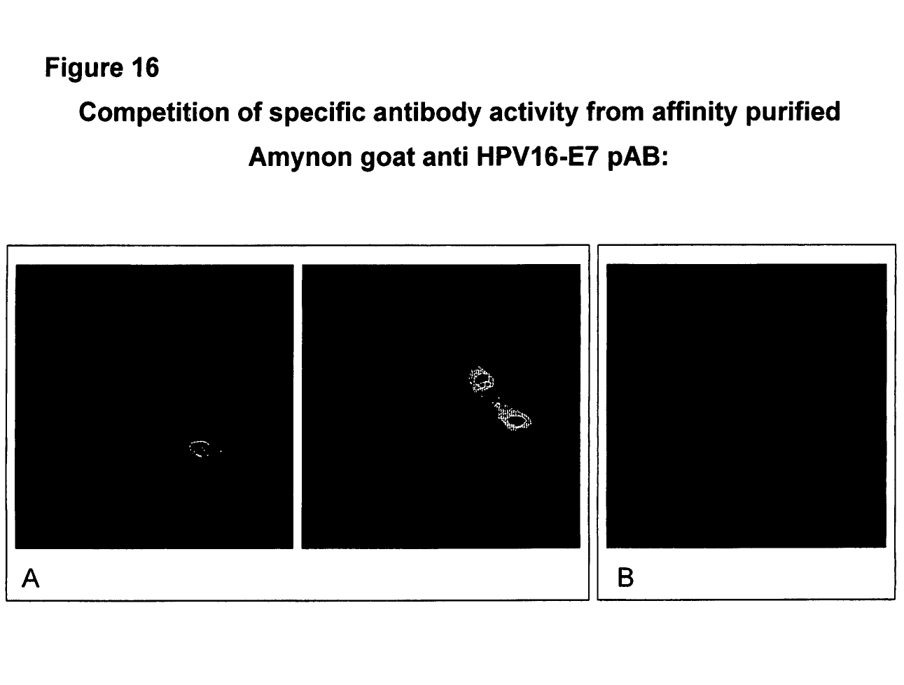

FIG. 16 Competition of specific antibody activity from affinity purified goat antiHPV-16 E7 antibody of the present invention. Panel A: no diminished signal was found after adsorption against GST- or GST-HPV-11 E7. Panel B: The signal was completely blocked after adsorbtion to GST-16 E7 protein.

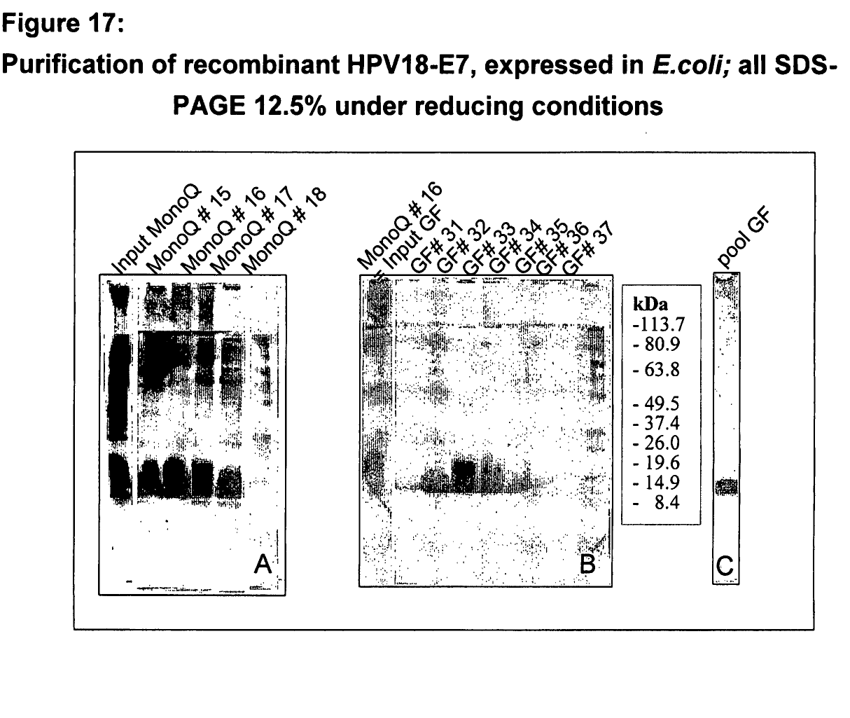

FIG. 17 Purification of recombinant HPV-18 E7, expressed in E. coli; all SDS-PAGES 12.5% under reducing conditions: A: IEC on MonoQ: input was AS precipitate of lysed Ecoli; elution in a linear NaCl gradient; fractions #15-18 contain mainly HPV-18 E7 protein and high molecular weight contaminants. B: Gelfiltration of MonoQ fractions #16. High MW contaminants were removed, material from pooled fractions #32-34 were used for immunization. Additional bands at 49.5 and 80.9 kDa derive from the sample buffer (DTT, beta Mercaptoethanol).

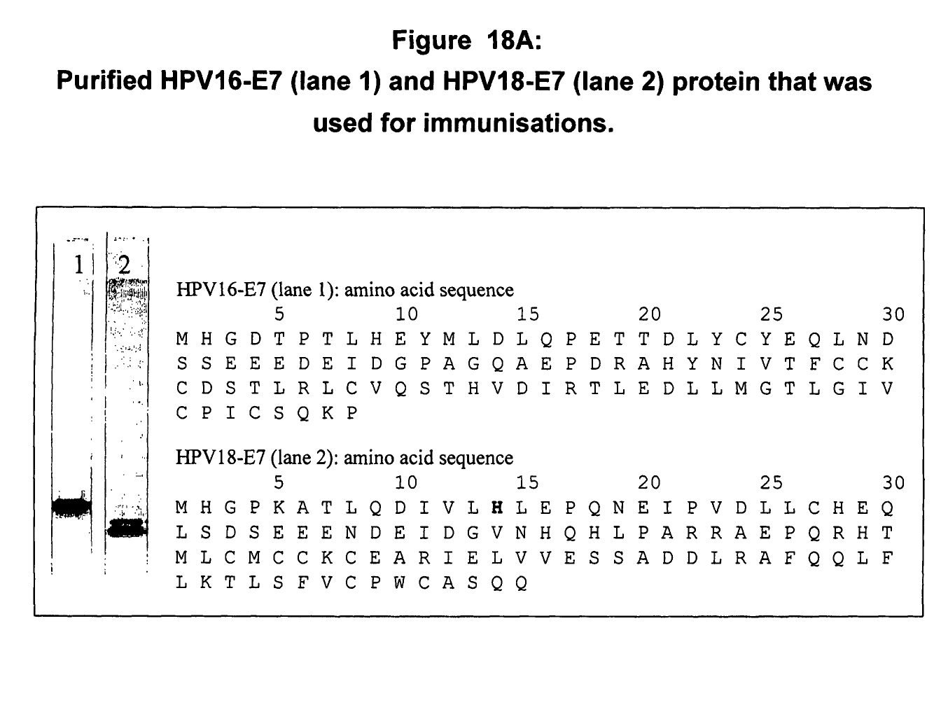

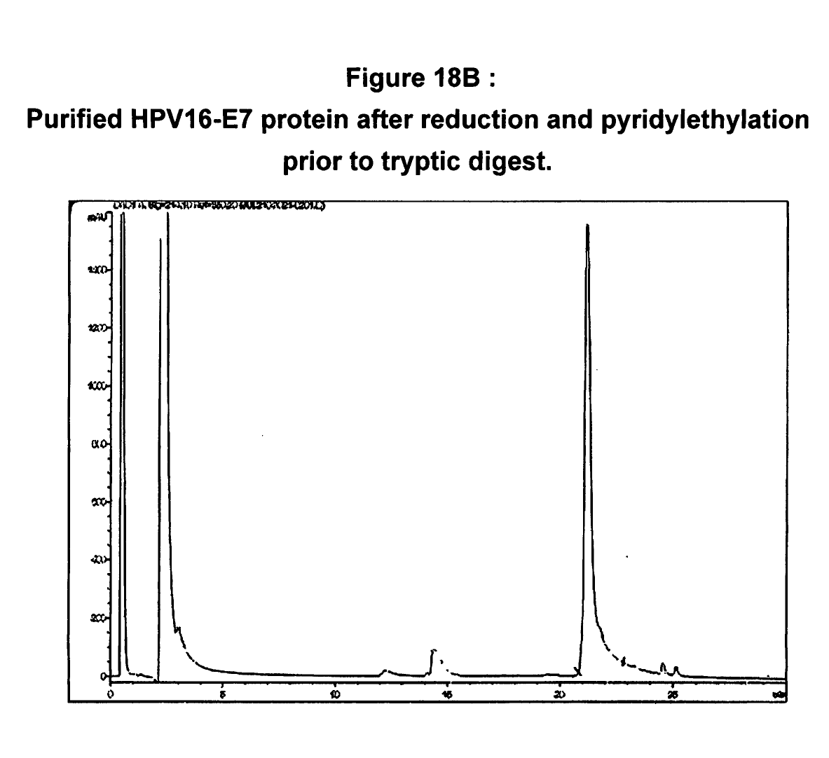

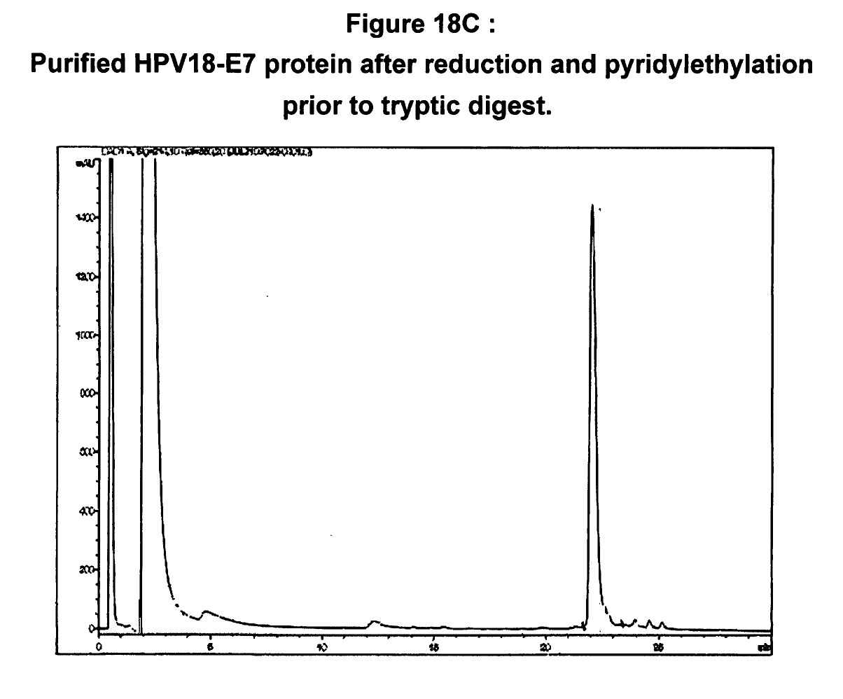

FIG. 18A Purified HPV-16 E7 (lane1) and HPV-18 E7 (lane 2) protein that was used for immunizations. The material was sequenced by Eurosequence B.V. (Netherlands); for results see example 2.3 and 5. 3 ‘Characterization of antigens used for immunization’. Proteins (5 μg per lane) were separated in 12.5% SDS-PAGE under reducing conditions. HPV-18 E7 shows two characteristic bands due to N-terminal degradation as confirmed by sequencing (a sub-fraction of the protein starts with a Histidine (position 14).

FIG. 18B Purified HPV-16 E7 protein after reduction and pyridylethylation prior to tryptic digest. The chromatogram shows the removal of salt and chemicals (initial peaks in minutes 1 and 3) after the modification process. The main peak in minute 21 represents highly pure material (see also lane 1 in FIG. 18A).

FIG. 18C Purified HPV-18 E7 protein after reduction and pyridylethylation prior to tryptic digest. The chromatogram shows the removal of salt and chemicals (initial peaks in minutes 1 and 3) after the modification process. The main peak in minute 22 represents highly pure material (see also lane 2 in FIG. 18A).

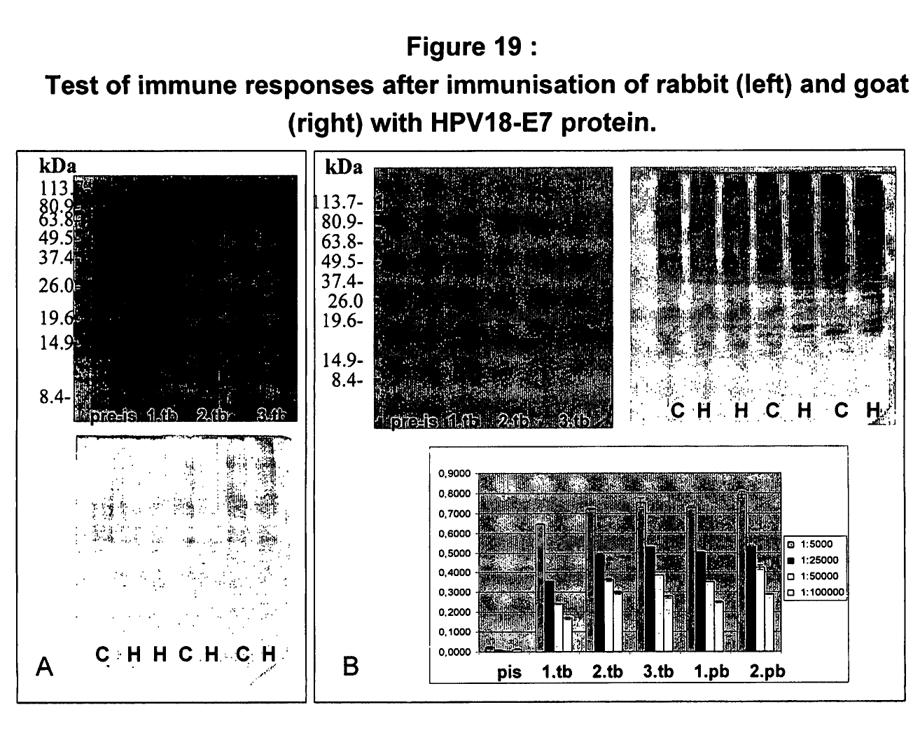

FIG. 19 Test of immune responses after immunization of goat and rabbit with HPV-18 E7 protein. W-blot from rabbit serum (panel A) and W-blot and ELISA from goat serum (panel B) are shown. C33A lysate (C)/HeLa lysate (H) (125 μg/lane) was probed with pre-immune (pis) and serum from 3 test-bleedings (tb). Coomassie stained, blotted gels are shown as input control. In the goat ELISA, serum samples from 1^{st} and 2^{nd} production bleeding are shown in addition.

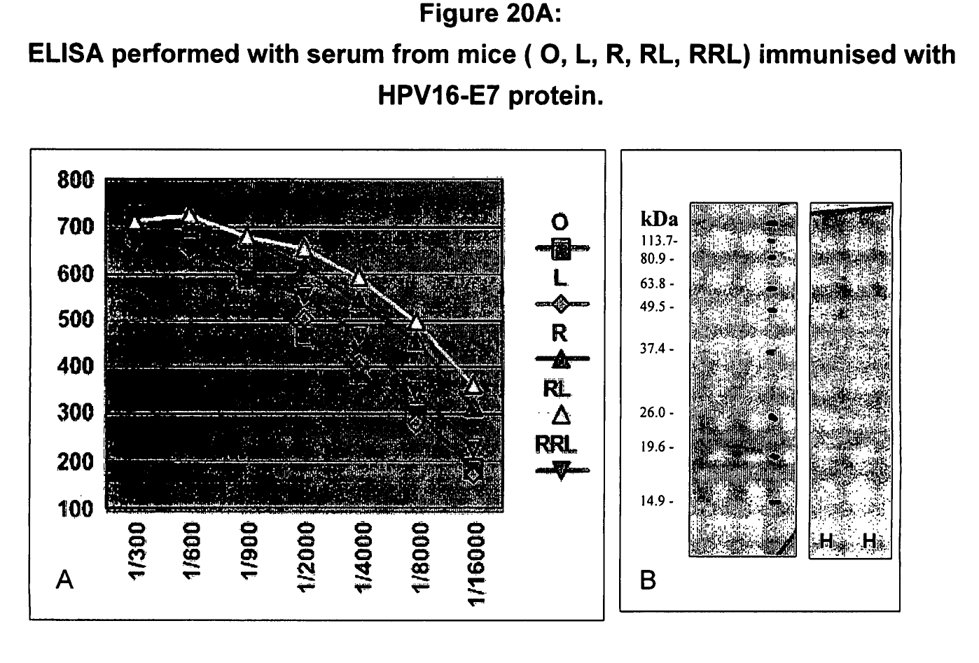

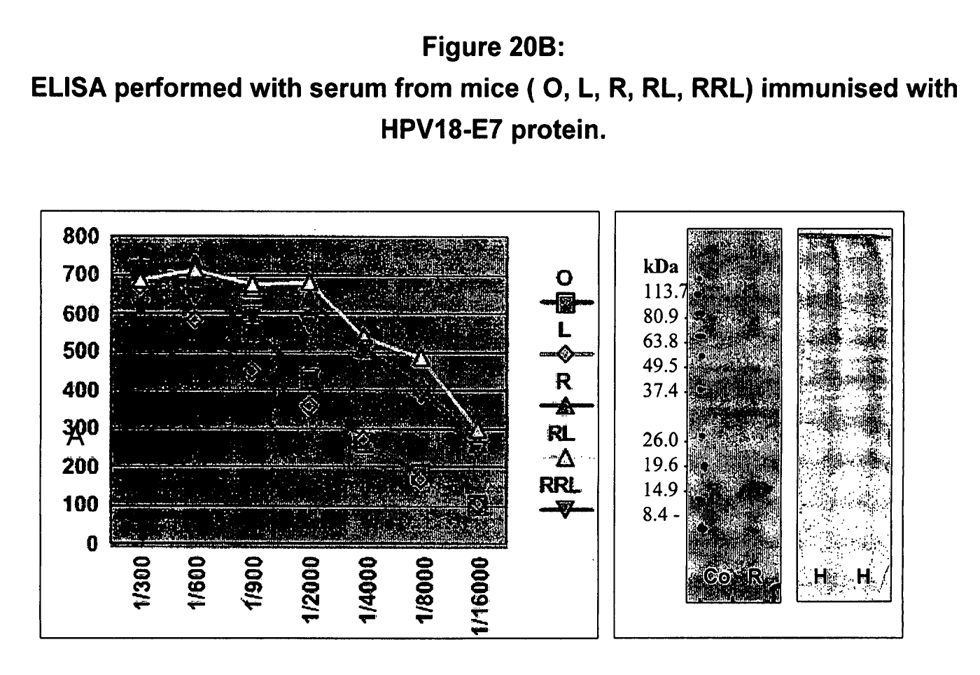

FIG. 20A Elisa performed with serum from mice (O, L, R, RL, RRL) immunized with HPV-16 E7 protein. Mouse serum was diluted 1:300 to 1:16000 (purified HPV-16 E7 was coated according to example 4.3); B: W-blot with serum from untreated mouse (Co) and serum from mouse R, immunized with HPV-16 E7 protein; serum were tested in a 1:400 dilution on NIH 3T3 E72 lysate 125 μg/lane (H); blotted and Coomassie blue stained gel is shown as input control.

FIG. 20B Elisa performed with serum from mice (O, L, R, RL, RRL) immunized with HPV-18 E7 protein. Mouse serum was diluted 1:300 to 1:16000 (purified HPV-18 E7 was coated according to example 4.3); B: W-blot with serum from untreated mouse (Co) and serum from mouse R, immunized with HPV-18 E7 protein; serum were tested in a 1:400 dilution on HeLa lysate 125 μg/lane (H); blotted and Coomassie blue stained gel is shown as input control.

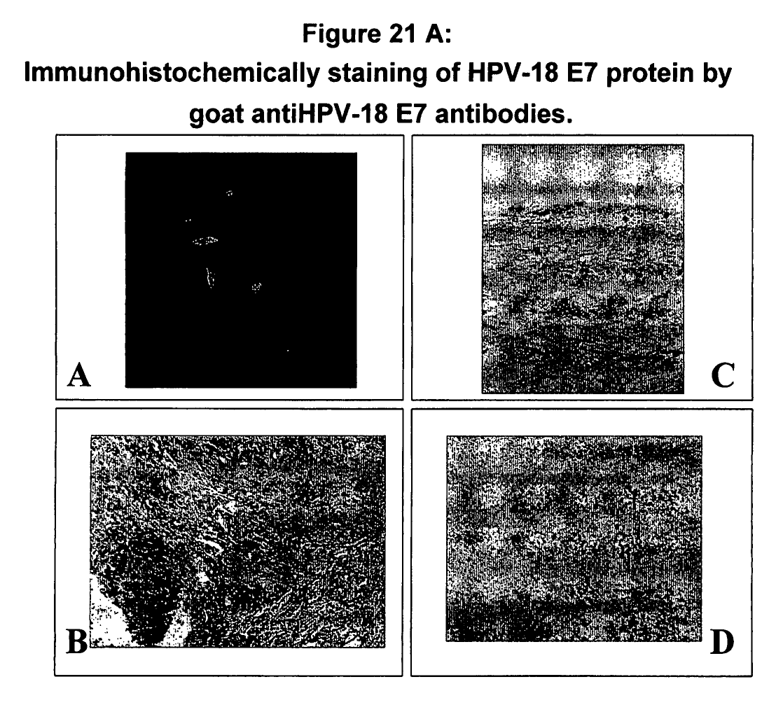

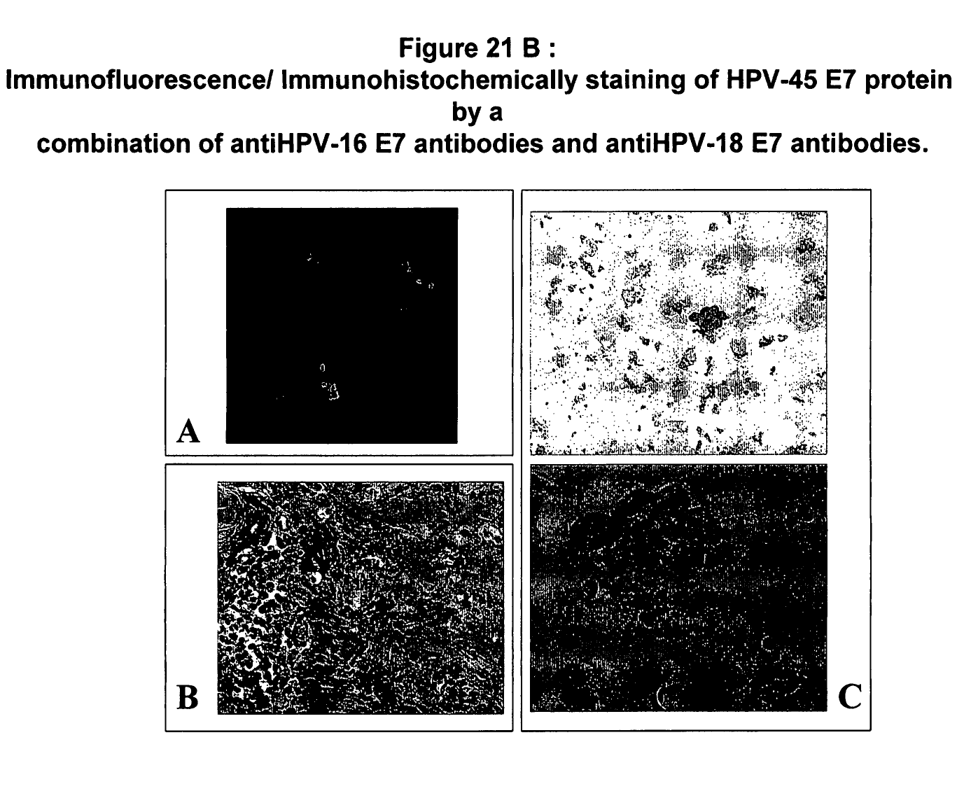

FIG. 21A Immunofluorescence/Immunohistochemically staining of HPV-18 E7 protein by goat antiHPV-18 E7 antibodies of the present invention. Panel A: IF-detection in HPV-18 E7 transiently transfected U2OS cells. Panel B: IHC. HPV-18 DNA positive cervical carcinoma, paraffin embedded tissue section. Panel C: no staining was found in normal squamous epithelium. Panel D: HPV-18 DNA positive cervical carcinoma, competition with HPV-18 E7 protein, the staining is completely deleted.

FIG. 21B Immunofluorescence/Immunohistochemically staining of HPV-45 E7 protein by a combination of goat antiHPV-16 E7 antibodies and antiHPV-18 E7 antibodies of the present invention. Panel A: IF-detection in HPV-45 E7 transiently transfected U2OS cells. Panel B: positive IHC staining in HPV-45 DNA positive cervical carcinoma, paraffin embedded tissue section. No staining is apparent in E7 negative stroma cells. Panel C: positive staining in cell monolayer präparation of Pap smear diagnosed with PapV, HPV-45 DNA positive.

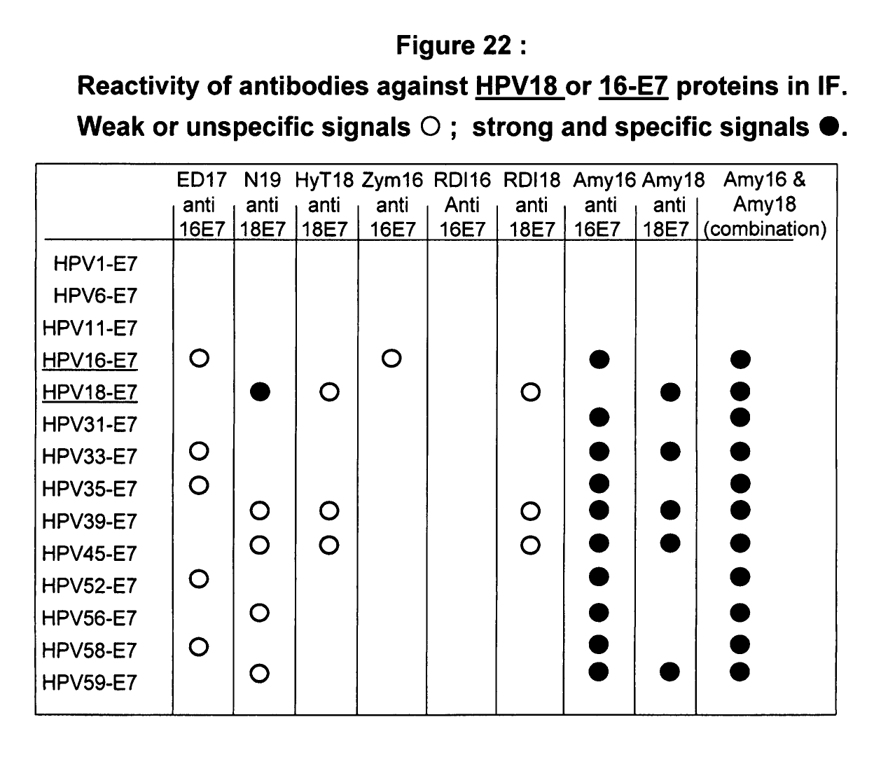

FIG. 22 Reactivity of different commercially available antibodies against HPV18 or 16 E7 proteins in IF in comparison to antiHPV-16 E7 antibodies and antiHPV-18 E7 antibodies of the present invention. Reactivity was tested against E7 proteins from high risk HPV types. Very weak or unspecific signals are marked with an empty circle, strong and specific signals are marked with an filled black circle. Beside antibodies of this invention none of the antibodies recognize HPV 31 E7 protein.

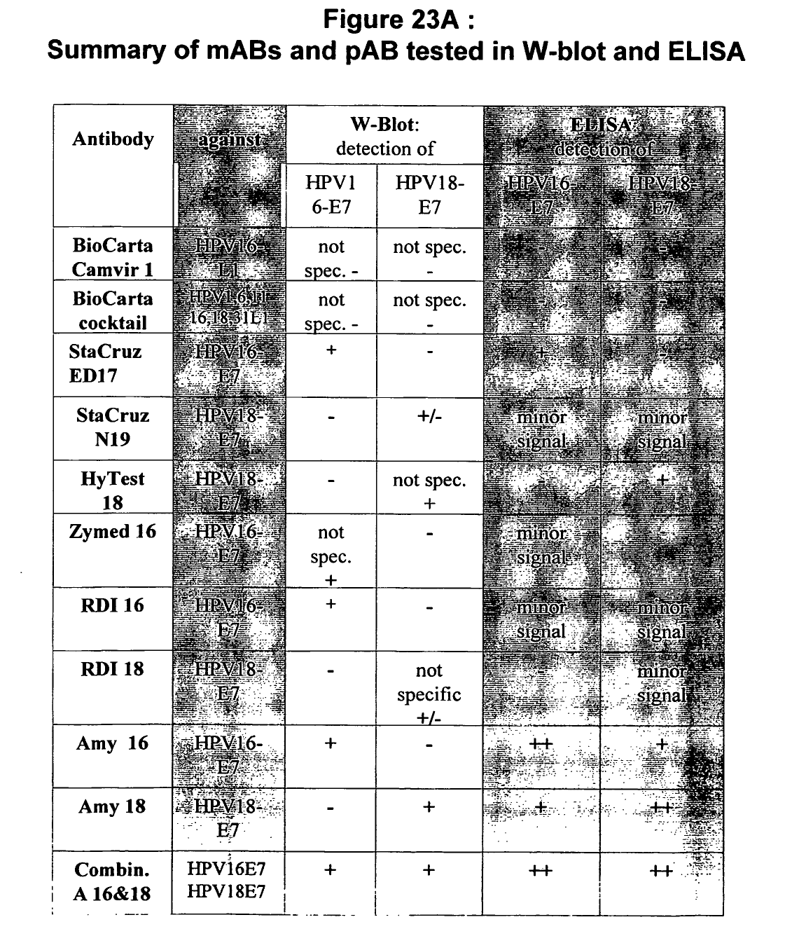

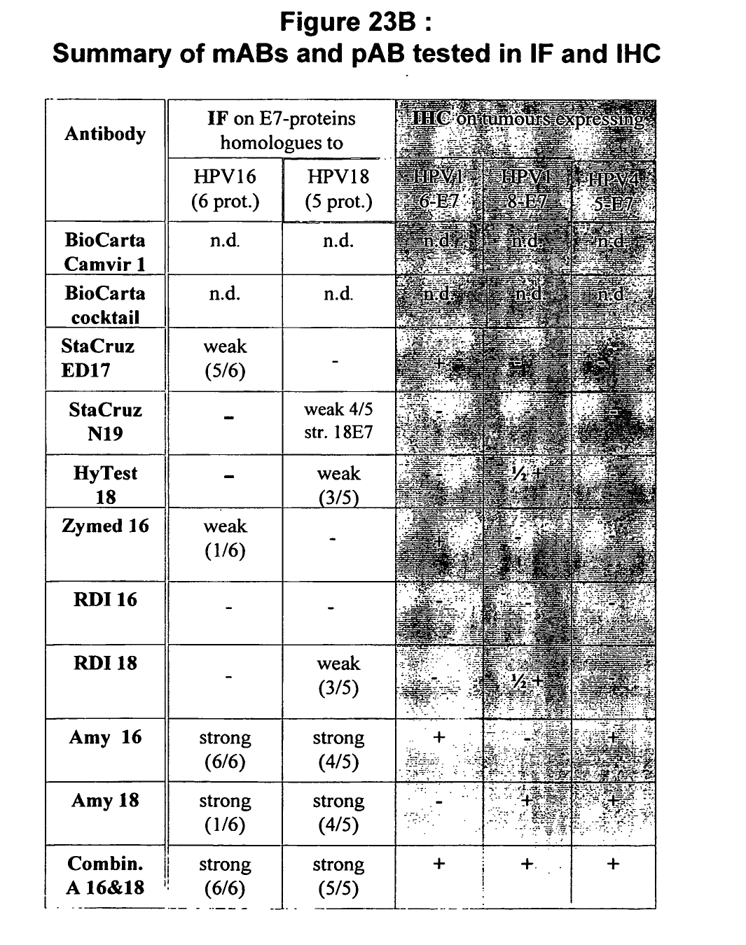

FIG. 23A Summary of mABs and pAB tested in W-blot and ELISA

FIG. 23B Summary of mABs and pABs tested in IF and IHC

The invention is illustrated by the following examples which are merely illustrative and are not constructed as a limitation of the scope of the present invention:

### EXAMPLE 1

### Construction of the Bacterial Expression Vector for HPV-16 E7

The HPV-16 E7 oncogene was amplified from the vector pX-HPV-16 E7 (Mannhardt et al., 2000) by PCR using Pfu DNA polymerase as EcoRI repair/BamHI fragment. The sequence was inserted into the bacterial expression vector pET3a (Studier and Moffatt, 1986) prepared as NdeI repair/BamHI fragment generating the bacterial HPV-16 E7 expression vector pET3a-HPV-16 E7/clone 17. The sequence encoding for HPV-16 E7 was verified by sequencing.

### EXAMPLE 2

### Expression and Purification of Recombinant HPV-16 E7 Protein

2.1. Expression of Recombinant HPV-16-E7 Protein

The expression vector pET3a-HPV-16 E7/clone 17 was transformed into E. coli strain BL21 (DE3) pLysS and preserved as glycerol stock. LB- or NZCYM-medium (25 ml) containing 100 μg/ml Ampicilline (Biomol, Hamburg, Germany;) and 25 μg/ml Chloramphenicol (Sigma, Vienna; Austria;) was inoculated with the glycerol stock and grown over night at 37° C. to a final OD₆₀₀ of 1.5. The next day NZCYB medium, containing 100 μg/ml Ampicillin and 25 μg/ml Chloramphenicol and 2 ml Glucose/I, was inoculated with 1% of the over night culture and grown at 37° C. to an OD₆₀₀ of 0.4. Culture volume was 400 ml per 2000 ml aeration flask. At OD₆₀₀=0.4 E7 expression was induced by adding IPTG (Biomol, Hamburg, Germany) to a final concentration of 0.4 mM. Two hours after induction bacteria were harvested by centrifugation for 10 minutes at 5 000×g. The drained cell pellets were either stored at −80° C. until further use (up to 3 month) or redissolved in ice-cold lysis buffer (50 mM KCl, 20 mM H₂KPO₄ [pH 7.8], 50 mM DTT, 5% glycerol, 1-μg/ml leupeptin, 1 mM PMSF, 1 mM NaF and 10 μg/ml Aprotinin) at a ratio of 2 ml fresh lysis buffer per pellet derived from 100 ml bacterial culture. When the pellet had been stored at −80° C. lysis buffer was added directly to the frozen material and cells were thawed on ice. For the following purification procedure two pellets from 400 ml E. coli culture each were used. Pellets were redissolved by repeated pipetting and lysed by sonication with glass beads (Sigma, Vienna, Austria) using a Sonifier 250 (Branson, Geneva, Switzerland) on ice.

The sonified lysate was centrifuged at 70 000 ×g for 1 hour and the supernatant stored on ice. The remaining pellet was redissolved in lysis buffer (again 2 ml fresh lysis buffer per pellet derived from 100 ml bacterial culture) and sonified and centrifugated as stated above. Supematants were pooled, cooled on ice and subjected to a two-step ammonium sulphate precipitation procedure.

2.2 Purification of Recombinant HPV-16-E7 Protein

To prepare a saturated Ammonium sulphate solution 75 g of (NH₄)₂SO₄ were added to 100 ml of 50 mM NaCl, 150 mM Tris/HCl pH 7.8. Ammonium sulphate was dissolved at RT and the saturated solution was cooled down on ice. (The cooled solution contained a few crystals of precipitated (NH₄)₂SO₄ indicating 100% saturation.) The saturated (NH₄)₂SO₄ solution was prepared freshly prior to use. The lysate was made 38% ammonium sulphate by adding 38 parts of cold, saturated (NH₄)₂SO₄ solution to 62 parts of cold lysate (19.6 ml saturated (NH₄)₂SO₄ to 32 ml lysate). The mixture was stirred gently on ice for 30 min and centrifuged at 4° C. for 30min at 30 000 ×g. After carefully discharging the supernatant, the pellet was dissolved in dialysis buffer (150 mM Tris/HCl pH 7.8, 10 mM NaCl, 10 mM DTT and 5% glycerol) at a ratio of 1 ml per pellet derived from 100 ml bacterial culture.

Dialysis was performed at 4° C. for 12 hours applying 3 buffer changes. Dialysis tubings with a molecular weight cut-off of 10 000 Dalton were used; the total volume of dialysis buffer was 250 times the sample volume. DTT was added prior to every buffer change. (For 8 ml of dissolved (NH₄)₂SO₄-pellet, 3×670 ml dialysis buffer were used). The dialysed probe was centrifuged at 10 000 ×g for 10 min and the supematant loaded (flow=1 m/1 min) onto a MonoQ HR 10/10 anion-exchange column (Amersham Biosciences, Vienna, Austria) equilibrated to 10% MonoQ buffer B (MonoQ buffer A: 150 mM Tris/HCl pH 7.8, 10 mM DTT (added prior to use) and 5% glycerol; MonoQ buffer B: 150 mM Tris/HCl pH 7.8, 1M NaCl, 10 mM DTT added prior to use) and 5% glycerol;). The MonoQ column was washed with 2 column volumes (CV) (flow=4ml/min) 10% buffer B, and eluted in multi step gradient at a flow rate of 2 ml/min: 10% -47% B (4 CV), 47% B (2CV), 47% -100% B (2 CV). At 47% buffer B (470 mM NaCl) E7 eluted in a prominent double peak over 4 fractions of 1 ml each. E7 containing fractions were individually loaded onto a HiLoad 16/60 Superdex 75 gel filtration column (Amersham Bioscienees, Vienna, Austria) and eluted at a flow rate of 0.5 ml/min with the gelfiltration buffer (150 mM Tris/HCl pH 7.8, 150 mM NaCl and 10 mM DTT (added prior to use)); fraction volume was 2 ml. E7 containing fractions from 4 runs were controlled on SDS-PAGE followed by Coomassie stain. E7 fractions of highest purity were pooled and the protein concentration was determined according to Bradford. The pool was diluted with gelfiltration buffer to a final concentration of 1 mg/ml and frozen in aliquots for further use. The total yield from 800 ml E. coli culture was approximately 14 mg of native, highly purified HPV-E7 in NMR-grade.

2.3. Characterisation of Antigen Used for Immunization

2.3.1. Purity of Antigen

SDS PAGE Gels were stained with Coomassie brilliant blue and the stained gel was evaluated by scanning, using the Adobe Photoshop Software and a MicroTec Scan Maker 8700 Image Scanning Device. This generated a profile of relative optical density which was used to determined the integral corresponding to the E7 peak. With this software it is possible to calculate the percentage of total OD units which are represented by the E7 peak. As judged by densitometrical analysis the E7 fractions resulting from this run were more than 98% pure (e.g. analyzed by silver-stained SDS-PAGE), or more than >99.5% pure (e.g. judged by Coomassie-stained SDS-PAGE) and were concentrated using a Centriprep10 ultrafiltration filter (Amicon, Vienna, Austria). The identity of the E7-protein was confirmed by Western Blot (FIG. 1B) and through a peptide mass fingerprint (PMF) (FIG. 1D).

Three different preparations of recombinantly expressed HPV 16 E7 protein were evaluated to document, the purity of the preparations and the reproducibility of the applied methods. Material generated on Aug. 8 2002 (used to immunise chinchilla rabbits; preparation “A”), and 2 production lots from Dec. 17 2002 (lot 1 used to immunise goats; lot 2 used to prepare an E7-affinity column; preparations “B” and “C”) were run on a 12.5% SDS-PAGE under reducing conditions (2.5% β-Mercaptoethanol). The amount of proteins separated per lane was 0.1 μg HPV16-E7, determined according to Bradford, using BSA as standard. The gel was silver stained according to Heukeshoven and Dernick (in R. Westermeier et al. 1990; ISBN 3-527-28172-X) for 30 min. The gel was scanned using a Fluor-S™ Multi-Imager system (BIORAD). Cross sections of defined lanes were saved as TIFF images using Quantity One (Quantitation Software by BIORAD). Evaluation of the gel-bands was performed by using TOTALLab evaluation software Version 1.1.

FIG. 1D shows the results from densitometric evaluation of three independent preparations A, B and C. Results were calculated from separation gels. A light background-staining in the stacking gel, derived from the sample buffer, was observed. Since the light background staining on top of the separation gel was found in every lane, it is assumed to be derived from an irrelevant compound from the sample buffer. Prior to evaluation, the background was subtracted from each lane separately. The sum of all pixels over the entire length of one lane was assumed to be equivalent to 100% of protein applied (0.1 μg/lane). Peaks were evaluated be recalculating the pixel-intensity of every protein band found into % of the total protein amount per lane. E7 concentration was 98.0% (A), 98.3% (B) and 98.2% (C). The curves shown in FIG. 1D are original traces from scanned lanes exported as MS-Excel files as the used set-up did not allow to print evaluated curves directly.

2.3.2. Secondary Structure of E7 Protein—Circular Dichroism spectroscopy (CD) Measurements of HPV-16 E7 Protein in Solution

Circular Dichroism (CD) is observed when optically active matter absorbs left and right hand circular polarized light slightly differently. CD spectra for distinct types of secondary structure present in peptides, proteins and nucleic acids are different. The analysis of CD spectra can therefore yield valuable information about secondary structure of biological macromolecules. In our case Circular Dichroism Spectroscopy is used to gain information about the secondary structure of native proteins and polypeptides in solution. The CD is a function of wavelength and is measured with the CD spectropolarimeter JASCO J-715. (See Circular Dichroism and Optical Rotary Dispersion of Proteins and Polypeptides, A. J. Alder, N. J. Greenfield and G. D. Fasman, Meth. Enzymology 27, 675 (1973)).

A preparation of recombinantly expressed and highly purified HPV-16 E7 protein as described herein was further evaluated for secondary structure elements that will occur in the native folded protein in a physiological solvent. Because of 7 Cysteins in the E7 molecule, the E7 protein tends to build di- and multimeres with proteins in vicinity by disulfide bridges. Patrick et al, 1992, JBC 265 (10):6910, describes, that no such disulfide-bonds exist inside the native E7 molecule. It was demonstrated that 3 cysteins are accessible to solvent, while cysteins in the two concerved Cys-X-X-Cys motifs are likely involved to be part of a zinc-finger motif. For this reason inclusion of a reducing substance in solvent like DTT (or 2-ME) results in monomeric native E7 protein particles (DTT and 2-ME do not have any denaturing effect). In addition, the amount of DTT in CD measurement is diluted to the lowest concentration that might be possible to adhere reducing conditions. Repeated measurements was carried out in 8 μl of a 50-100 μmolar protein solution in NMR buffer (20 mM H₂KPO₄, 50 mM KCl, 10 mM NaCl, 10 mM DTT, pH 7.5) diluted in in 80 μl of a. dest.

The obtained measurement data were interpreted by the calculation program of the CD spectropolarimeter JASCO H-715 (H-700 Secondary Structure Estimation for Windows, version 1.10.02, Jasco). The CD-spectrum and structural data are shown in FIG. 1E. The HPV-16 E7 protein is folded into secondary structure elements like β-sheets (45-47%), coils (40-43%), α-helices (7-8%) and turns (3-5%).

2.3.3. Characterization by N-terminal Sequencing

Purified HPV16-E7 protein that was used for the immunisation of goats, rabbits and mice was characterize by Eurosequence b.v. (Meditech Center L.J. Zielstraweg 1, 9713 GX Groningen The Netherlands). Eurosequence b.v. reference number is 040705/758/cc. For reference numbers of individual result-files see below. FIG. 18A shows aliquots of the material send to Eurosequence b.v. separated in 12.5% SDS-PAGE under reducing conditions.

Proteins were N-terminally sequenced and digested by trypsin. The fragments were also sequenced to verify the correctness of the peptides. To allow fragmentation, proteins were chemically modified (see below) followed by the removal of salt and modifying agents through RP-HPLC. FIG. 18B. (HPV16-E7) show the chromatograms of the removal procedure. The profile shows 2 peaks in the first 5 minutes, corresponding to salt and chemicals eluting from the column. The main peaks in minutes 21 represent highly purified HPV16-E7 (see also FIG. 18A). Antigen and fragments were sequenced by Edman degradation with an automated sequenator (Model 494 Procise Applied Biosystems); For N-terminal sequencing 8 cycles were done

N-terminal Sequencing (Eurosequence Result File 04C313)

As confirmed by N-terminal sequencing, the main sequence is 100% in agreement with the expected N-terminal sequence of the E7 protein of human papilloma virus type 16. The main sequence was: (Met)-(His)-(Gly)-(Asp)-(Thr)-(Pro)-(Thr)-(Leu). One of the minor signals at each position was brought into agreement with the n-2 mer of the protein: (Gly)-(Asp)-(Thr)-(Pro)-(Thr)-(Leu)-(His)-(Glu); this was approx. 20% with regard to the main sequence. The remaining minor signals were brought into agreement with the n-1 mer of the protein: (His)-(Gly)-(Asp)-(Thr)-(Pro)-(Thr)-(Leu)-(His); this was approx. 7% with regard to the main sequence. To conclude, 73% of the sample are full length HPV16-E7, 7% are degraded at the N-terminus missing one amino acid (Met) and 20% are degraded at the N-terminus missing two amino acids (Met, His).

Sequencing Yield and Result Was:

| | |
|---|---|
| position: | 1 5 |
| 83% | (Met)-(His)-(Gly)-(Asp)-(Thr)-(Pro)-(Thr)-(Leu)-............................... |
| 7% | (Gly)-(Asp)-(Thr)-(Pro)-(Thr)-(Leu)-(His)-(Glu)-................ |
| 20% | (His)-(Gly)-(Asp)-(Thr)-(Pro)-(Thr)-(Leu)-(His)-........................ |

Result: 100% of the sequenced protein is HPV16-E7 as confirmed by a data-base search. The protein was identified as:

| | |
|---|---|
| AAL96649 | E7 protein [Human . . . [gi:19744738] |
| LOCUS | AAL96649 98 aa linear VRL 12-AUG-2002 |
| DEFINITION | E7 protein [Human papillomavirus type 16]. |
| ACCESSION | AAL96649 |
| VERSION | AAL96649.1 GI:19744738 |
| DBSOURCE | accession AF486344.1 |
| SOURCE | Human papillomavirus type 16 |
| ORGANISM | Human papillomavirus type 16 |
| | Viruses; dsDNA viruses, no RNA stage; Papillomaviridae; |
| | Papillomavirus. |
| REFERENCE 1 | (residues 1 to 98) |
| AUTHORS | Chan, P. K. S., Lam, C. W., Cheung, T. H., Li, W. W. H., Lo, K. W. K., |
| | Chan, M. Y. M., Cheung, J. L. K., Xu, L. Y. and Cheng, A. F. |
| | TITLEHuman papillomavirus type 16 intratypic variant infection |
| | and risk for cervical neoplasia in Southern China |
| JOURNAL | J. Infect. Dis. 186 (5), 696-700 (2002) |
| REFERENCE 2 | (residues 1 to 98) |
| AUTHORS | Chan, P. K. S., Lam, C. W., Cheung, T. H., Li, W. W. H., Lo, K. W. K., |
| | Chan, M. Y. M., Cheung, J. L. K., Xu, L. Y. and Cheng, A. F. |
| TITLE | Direct Submission |
| JOURNAL | Submitted (22-FEB-2002) Department of Microbiology, The |
| | Chinese |
| | University of Hong Kong, Prince of Wales Hospital, Shatin, N. T., |
| | Hong Kong SAR, China |

The sequence of HPV16-E7 is:

```
[SEQ ID NO:2]
                      5 10 15 20 25 30
  1 M H G D T P T L H E Y M L D L Q P E T T D L Y C Y E Q L N D
31 S S E E E D E I D G P A G Q A E P D R A H Y N I V T F C C K
61 C D S T L R L C V Q S T H V D I R T L E D L L M G T L G I V
91 C P I C S Q K P
```

2.3.4. Characterization by Tryptic Digest and Identification of Fragments by RP-HPLC and Sequencing Eurosequence Result File 04E168, 04E174, 04C331 and 04E175)

Prior to trypsin digestion it was necessary to subject the sample to a reduction and pyridylethylation step as cystein residues had to be reduced and blocked to inhibit formation of thiol-bridges. HPV16-E7 was digested by trypsin and the resulting fragments were N-terminally sequenced and subjected to a peptide profiling. In accordance with theoretical cleavage products at amino acid positions 49, 60, 66 and 77 (http://www.expasy.org/tools/peptidecutter/) 5 fragments were obtained, separated by RP-HPLC and sequenced. Results of 6 sequencing cycles were:
cycle 1: (Met/Leu/Cys/Ala/Thr)=1^{st} amino acids of 5 fragments respectively
cycle 2: (Asp/Cys/Leu/His)=2^{nd} amino acids of 5 fragments respectively
cycle 3: (Gly/Ser/Glu/Tyr/Val)=3^{rd} amino acids of 5 fragments respectively
cycle 4: (Gln/Asn/Thr/Asp)=4^{th} amino acids of 5 fragments respectively
cycle 5: (Ile/Leu/Thr/Ser)=5^{th} amino acids of 5 fragments respectively
cycle 6: (Thr/Arg/Val/Leu/Pro)=6^{th} amino acids of 5 fragments respectively

The interpretation of the obtained fragments on the basis of known sequence information allows the following conclusion.

| | |
|---|---|
| fragment 1 (N-terminus): | (Met)-(His)-(Gly)-(Asp)-(Thr)-(Pro)............... |
| fragment 2 (cleaved at AA 49): | (Ala)-(His)-(Tyr)-(Asn)-(Ile)-(Val)................. |
| fragment 3 (cleaved at AA 60): | (Cys)-(Asp)-(Ser)-(Thr)-(Leu)-(Arg)............... |
| fragment 4 (cleaved at AA 66): | (Leu)-(Cys)-(Val)-(Gln)-(Ser)-(Thr)............... |
| fragment 5 (cleaved at AA 77): | (Thr)-(Leu)-(Glu)-(Asp)-(Leu)-(Leu).............. |

Taken all information together the HPV16-E7 protein was verified by sequence analysis of the underlined amino acids.

```
[SEQ ID NO:2]
                      5 10 15 20 25 30
  1 M H G D T P T L H E Y M L D L Q P E T T D L Y C Y E Q L N D
31 S S E E E D E I D G P A G Q A E P D R A H Y N I V T F C C K
61 C D S T L R L C V Q S T H V D I R T L E D L L M G T L G I V
91 C P I C S Q K P
```

HPV16 E7 protein characterised in 2.3. was used for immunisation of goat, rabbit and mice.

### EXAMPLE 3

### Generation, Purification, Quality Controls and Characterization of Polyclonal HPV-16 E7 Antibodies from Rabbit

3.1. Generation of Polyclonal HPV-16 E7 Antibodies in Rabbit

Purified preparations of the HPV-16 E7 protein were used to produce highly specific polyclonal anti-HPV-16 E7 antibodies in chinchilla bastard rabbits (Charles River, Germany). 1^{st} injection: 700 μl complete Freund's adjuvant (Sigma, Vienna, Austria) was mixed with 500 μg HPV-16 E7 protein dissolved in 700 μl PBS by sonication (Branson sonifier 250, level 5-7, 3×10 seconds). A total of 300 μg HPV-16 E7 protein was injected. 1^{st} boost: 32 days after the first injection, 500 μl incomplete Freund's adjuvant was mixed with 500 μg 16 E7 protein dissolved in 500 μl PBS by sonication. A total of 500 μg HPV-16 E7 protein was injected. 2^{nd} boost: 28 days after the first boost, 500 μl incomplete Freund's adjuvant was mixed with 500 μg 16E7 protein dissolved in 500 μl PBS by sonication and a total of 500 μg HPV-16 E7 protein was injected. 3^{rd} boost: 27 days after the second boost, 500 μl incomplete Freund's adjuvant was mixed with 500 μg 16 E7 protein dissolved in 500 μl PBS by sonification. A total of 500 μg HPV-16 E7 protein was injected. Bleeding was done 10 days after the third boost. In particular, small aliquots of sera were tested in western blot 10 days after the first, second and third boost (second, third and forth injection). A first and clear signal was obtained after the third boost.

TABLE 1
Timetable to generate polyclonal antibodies in rabbit.

| day | application of HPV16 E7 | bleedings |
|---|---|---|
| −3 | | pre-immune |
| | | serum taken |
| 1 | immunisation with 500 μg 16 E7 incomplete FA | |
| 33 | 1^{st} boost with 500 μg 16 E7 in incomplete FA | |
| 43 | | 1^{st} test bleeding |
| 61 | 2^{nd} boost with 500 μg 16 E7 in incomplete FA | |
| 71 | | 2^{nd} test |
| | | bleeding |
| 88 | 3^{rd} boost with 500 μg 16 E7 in incomplete FA | |
| 98 | | final bleeding |

A good immune response was also achieved by using 150 μg and 300 μg HPV16 E7 as antigen respectively. The immunisation schedule was as stated above.

3.2. Affinitypurification of Polyclonal HPV-16 E7 Antibodies

Three different columns were used to purify polyclonal HPV-16 E7 antibodies from animal serum by affinity chromatography.

Column 1: (column to purify total IgG from antiserum). A HiTrap Protein G HP column (Amersham Biosciences, Vienna, Austria) was used according to the manufacturers protocol to isolate total IgG from antiserum.

Column 2: (pre-column without antigen to adsorb unspecific antibodies to the affinity matrix): 2.8 g of freeze dried CNBr-activated sepharose 4B (Amersham Bioscieces, Vienna, Austria) were activated according to the manufacturers protocol and transferred into coupling buffer (100 mM NaHCO₃, 500 mM NaCl, pH 8.3) containing 13 mg of NIH 3T3 fibroblasts cell lysate (determined according to Bradford). Coupling was performed for 2 hours at room temperature in a 50 ml Falcon tube attached to a rotating platform. Once coupling was completed, the affinity matrix was packed into a XK16 FPLC column (Amersham Biosciences, Vienna, Austria) by gravity. The settled gel-bed (10 ml) was then washed with 5 column volumes of coupling buffer and 5 column volumes of blocking buffer (1M ethanolamine, pH 8.0). The column was then left at room temperature for 2 hours with out agitation to block remaining active groups, and thereafter washed with 5 column volumes high pH buffer (100 mM Tris/HCl, 500 mM NaCl, pH 8.0) and 5 column volumes low pH buffer (100 mM Na-acetate, 500 mM NaCl, pH 4.0). The cycle high pH-wash/low pH-wash was repeated 5 times. Finally the column was attached to the FPLC system and equilibrated to running buffer (PBS, 200 mM NaCl, 5 mM EDTA, 0.05% NaN3, pH 7.4). Protein contents (Bradford) of coupling buffer before and after coupling, and of all through-runs and wash buffers collected, revealed a coupling efficiency of approximtatly 80% of NIH-3T3 proteins to the column. The ligand density was 1 mg/ml gel-bed; the column volume was 10 ml.

Column 3: (affinity column, carrying purified HPV16-E7 protein to isolate polyclonal HPV16-E7 antibodies). As affinity matrix CNBr-activated Sepharose 4B (Amersham Biosciences, Vienna, Austria) was used. Preparation of the column was as stated above (column 2), but with recombinant, purified HPV16-E7 (examples 2A-2C) used as ligand. Prior to coupling, the ligand was dialysed (from 150 mM Tris/HCl, 150 mM NaCl, 10 mM DTT, pH 7.8) into coupling buffer as Tris would interfere with the coupling procedure. The optimal ligand density for affinity purification was found to be 1 mg antigen per ml gel-bed. For the experiment described below, an affinity column of a bed-volume of 3 ml, carrying 1.5 mg of HPV16-E7 was used.

Purification of the Polyclonal HPV16-E7 Antibody:

Antiserum was diluted 1+9 in running buffer (PBS, 200 mM NaCl, 5 mM EDTA, 0.05% NaN₃, pH 7.4). Diluted material was filtered trough a 0.45 μm sterile filter and passed over a 1 ml Protein G column using an Akta Prime system (Amersham Biosciences) at a flow rate of 1 ml/min . The column was extensively washed with running buffer until the baseline was zero (5 ml/min). Total IgG was eluted in 1 ml fractions (1 ml/min) with 100 mM Glycine, 0.05% NaN₃, pH 2.5 into 1.5 ml reaction vials containing 50 μl of 3 M KH₂PO_{4/}K₂HPO₄-buffer pH 7.4 to neutralize the low pH of the elution buffer.

IgG containing fraction (4×1.050 ml) were pooled, topped up to 10 ml with running buffer and loaded onto column 2 equilibrated with 10 column volumes of running buffer. Material was passed over the pre-column at a flow rate of 5 ml/min for 60 min in a closed circle to remove antibodies that would bind unspecifically to the CNBr activated sepharose matrix, and to immobilised proteins other than HPV16-E7. Thereafter the adsorbed material was collected (still 10 ml) and pooled with 5 column volumes of running buffer used to wash loosely bound, but probably specific antibodies from the pre-column. Finally the material (15 ml) was passed over the affinity column (column 3, equilibrated in running buffer) at a flow rate of 5 ml/min in a closed circle until an equilibrium was reached. The column was then washed (5 ml/min) with PBS, 1M NaCl, 5 mM EDTA, 0.05% NaN₃, pH 7.4 until the baseline reached zero. After re-equilibration into running buffer (10 column volumes), polyclonal anti HPV16-E7 antibodies were eluted (1ml/min) with 100 mM Glycine, 0.05% NaN₃, pH 2.5 into 1.5 ml reaction vials containing 50 μl of 3 M KH₂PO4/K₂HPO₄ buffer to neutralize the low pH of the elution buffer (fraction size was 1 ml). After elution, the column pH was set back to neutral by passing 10 column volumes of 1M Tris/HCl pH 7.4, 10 column volumes of 3 M KSCN, 150 mM KCl, 10 mM KH₂PO₄K₂HPO₄, pH 7.4 and 20 column volumes of running buffer through the system.

Anti HPV16-E7 antibody containing fractions were pooled and tested further like stated below. It was found that the pre-column (column 2, carrying NIH-3T3 cell lysate as ligand) in some cases could be omitted. Pooled eluates from column 1 (protein G column) were diluted 1+9 in running buffer and directly applied to column 3 (affinity column).

A further protocol for affinity purification of polyclonal HPV-16 E7 antibodies in small batches comprises the following:

Glutathione-S-transferase (GST-HPV-16 E7) and GST (control) were expressed from the expression vectors pGEX4T-GST-HPV-16 E7 and pGEX4T (Mannhardt, 2000; Mol Cell Biol 20:6483-95) in the E. coli strain DH5a. Expression was induced by adding IPTG to a final concentration of 1 mM to a 200 ml bacterial culture at OD₆₀₀=1.0. The bacteria were washed once in PBS and lysed in PBSDT (1.5 mM KH₂PO₄, 8.1 mM Na₂HPO₄ [pH 7.4], 2.7 mM KCI, 137 mM NaCI, 0.2 mM phenylmethylsulfonyl fluoride [PMSF], 1 mM NaF, 1 mM dithiothreitol [DTT] and 0.5% Triton X-100) by sonication using a Branson sonifier 250. The lysates were centrifuged at 4 000 ×g for 10 minutes and afterwards at 30 000 ×g for 30 minutes to remove the cell debris. The recombinant proteins were purified by affinity-chromatography using the glutathione sepharose 4B system (Amersham, Vienna, Austria). Clear supematants were incubated for 3 hours at 4 C with 150 μl glutathione sepharose 4B beads, which were prior, equilibrated in cold (4 ° C.) PBSDT. After the binding interval the beads were washed 4 times in 5 ml of PBSDT and stored at 4° C. in PBSDT. Purity of the preparation was controlled by western blotting using an anti E7 antibody (clone ED17, Santa Cruz, Vienna, Austria) and by Coomassie staining. Aliquots of 200 μg of bound GST proteins were separated on a 12.5% SDS-PAGE and transferred to a polyvinylidene difluoride (PVDF) membrane (NEN, Boston, USA) by electro blotting. PVDF membranes were dried and the proteins were crosslinked to the membrane by UV irradiation. The protein bands were stained with Ponceau S solution, excised from the PVDF membrane, destained and transferred to microfuge tubes.

Subsequently, the fragments were incubated with the polyclonal rabbit HPV-16 E7 antiserum for 2 hours at room temperature and washed 3 times in PBS-T (1.5 mM KH₂PO₄, 8.1 mM Na₂HPO₄ [pH 7.4], 2.7 mM KCl, 137 mM NaCl and 0.5% (vol/vol) Tween 20). Each fragment was then eluted by three 30 seconds washes with 5 mM glycine-HCl, [pH 2.3], 500 mM NaCl, 0.5% (vol/vol) Tween 20, 10 g/ml BSA in volumes of 500 μl; these eluates were immediately neutralized by the addition of Na₂PO₄ to a final concentration of 50 mM. The purified antibodies were concentrated 4 times using a centriprep YM-3 centrifugal filter (Millipore Corporation, Bedford, USA). After the pH 2.3 elution, fragments were further washed with three similar aliquots of PBS-T and 10 μg/ml of BSA, followed by three washes with 3 M NH₄SCN, 150 mM KCl, 10 mM NaPO₄ [pH 6.0], and 10 mg/ml BSA. The procedure was repeated 5 times.

### EXAMPLE 3.3.

### Test of the Affinity Purified HPV-16 E7 Antibodies by HPV-16 E7 Detection

The affinity purified HPV-16 E7 antibodies from rabbit specifically recognize HPV-16 E7 in cell lysates from HPV-16 E7 expressing mammalian cells in western blot experiments (FIG. 2B). HPV-16 E7 was also detectable in human U-2-OS cells transiently transfected with a HPV-16 E7 expression vector by indirect immunofluorescence microscopy using the confocal scanning system (FIG. 3). Furthermore, the antibodies recognize HPV-16 E7 in immunohistochemical experiments done in paraffin-embedded sections of cervical carcinomas derived from biopsies of HPV-16 positive patients (FIG. 4A-D). Biopsies from 12 carcinoma patients which had been classified by PCR-methods as “HPV-16 positive” were analyzed and positive signals were obtained with the E7 antibody in all 12 cases. Furthermore, in two cases of cervix biopsies which had previously been classified by PCR-methods as “HPV-16 negative”, the antibody described herein was able to specifically detect expressed E7.

### EXAMPLE 3.4.

### Western Blot (Immunoblot) Analysis

Cell extracts were separated on a 12.5% sodium dodecyl sulfate (SDS)-polyacrylamide gel, and proteins were transferred to a PVDF membrane (NEN, Boston, USA). The membrane was incubated in blocking buffer (0.05% Tween 20/5% low fat milk powder in PBS) for 1 hour at room temperature, washed in blocking buffer and incubated with the first antibody (affinity purified polyclonal rabbit anti-HPV-16 E7 antibody) for 1 hour at room temperature. After washing in blocking buffer, PBS/0.05% Tween 20 and PBS/5% low fat milk powder the membrane was incubated with the second antibody (peroxidase-conjugated anti-rabbit IgG, Promega, Mannheim, Germany) for 45 minutes at room temperature. The membrane was washed, and the bound antibodies were visualized by using the cherniluminescence Western blotting detection system (NEN, Boston, USA).

### EXAMPLE 3.5.

### Indirect Immunofluorescence Analysis

U-2-OS cells were cultured in DMEM+10% FCS. For transient expression of cDNAs, cells were grown to about 80% confluence on glass coverslips coated with 0.05% gelatin. Transfection of the expression vector pJ4HPV-16 E7 (Massimi, et al., (1997) J. Gen. Virol. 78, 2607-2613) was performed by using Effectene (Qiagen, Hilden, Germany). 24 h post-transfection, cells were prepared for indirect immunofluorescence according to standard protocols, including methanol fixation. After incubation with the primary antibody (affinity purified polyclonal rabbit anti-HPV-16 E7 antibody), and secondary antibody (FITC-conjugated anti-rabbit IgG, Dianova, Hamburg, Germany), cells were washed and embedded in Fluoromount G (Biozol, Eching, Germany). Samples were viewed by indirect immunofluorescence microscopy using the confocal scanning system MicroRadiance (Bio-Rad, Munich, Germany) in combination with a Zeiss Axiophot microscope. The following filters were used for FITC-derived fluorescence: excitation at 488 nm, emission at 515-530 nm). In these experiments, HPV-16 E7 were detected in 5-10% of the transfected cells, whereas no signal was obtained in cells that have been transfected with the empty expression vector. This result clearly proves that the antibody is specific for the E7 protein and does not detect any non-specific background under these conditions. The superior properties of the antibodies of the invention can be furthermore illustrated in the following experiment: To calibrate the antibodies, human U2OS cells were transfected with a CMV-driven expression vector for HPV-16 E7 and the staining of transfected cells by the antibodies as described herein was compared to the staining pattern obtained with commercially available antibodies from SantaCruz Biotechnology (ED17) or from Zymed Laboratories (8C9X). Staining was analysed by indirect immunofluorescence and evaluated by confocal microscopy. In these experiments, the commercially available antibodies (obtained from SantaCruz Biotechnology and Zymed Laboratories) were employed in accordance with manufacturers recommendations and gave high, unspecific background staining in all cells. Yet, no specific signal for detection of E7 antigene in the transfected cells could be obtained with these prior art antibodies. The corresponding results are documented in appended FIG. 9. In contrast, the antibodies according to the invention are able to specifically detect expressed E7 (positive signals in 5-10% of the transfected cells) and reveal no signal in cells transfected by an empty expression vector. These results indicate that the antibodies described herein recognize only the E7 protein, whereas the commercially available antibodies used in the study recognize unrelated antigens in the preparation. When tested in immunohistochemical stainings, there was a high and apparently non-specific background obtained with the antibodies obtained from SantaCruz Biotechnology or Zymed Laboratories in tissues derived from cervix carcinoma patients, as well as in tissue derived from normal cervix. Furthermore, the positive signals obtained with the SantaCruz antibodies were not restricted to the area that is cytologically recognized as tumor tissue, but were also present in the non-tumor tissue. In contrast to these results, staining of the biopsy material by the antibody according to the invention yielded positive results only for HPV-16 positive patients. As can be seen in FIGS. 4 and 8, staining was clearly confined to the area of the tumor.

### EXAMPLE 3.6.

### Immunohistochemical Detection of HPV-16 E7 in Biopsies Derived from Cervical Carcinomas

Immunohistochemistry was performed on paraffin-embedded sections of HPV16 positive biopsies derived from cervical carcinomas and control tissue specimens.

The paraffin-embedded tissue specimens were sectioned at 5 μm. Sections were mounted on slides, deparaffinized in xylol (2×10 minutes), incubated for 5 minutes each in 100%, 90%, 80% and 70% ethanol and blocked in 5% H₂O₂ in absolute methanol for 15 minutes. Before immunostaining the sections were washed twice in TRIS buffer (7.75 g Tris-HCl pH 7.5, 8.78 g NaCl ad 1 liter aqua dest) and processed for a 15 minutes blocking reaction in diluted (1:10 in TRIS buffer/1% BSA) goat-serum (DAKO, Hamburg, Germany). Sections were washed in TRIS/1% BSA buffer and incubated with the first antibody (affinity purified polyclonal rabbit anti-HPV-16 E7 antibody) for 1 hour at room temperature in buffer B (10 μg/ml BSA/10 μg/ml NIH3T3 lysate in PBS). The samples were rinsed twice in TRIS/1% BSA buffer and incubated for 1 hour at room temperature with the second antibody (Biotin-conjugated anti-rabbit IgG, DAKO, Glostrup, Denmark). After the washing step in TRIS/1% BSA, ExtrAvidin-conjugated peroxidase solution (Amersham Biosciences, Vienna, Austria) was added and the samples were incubated for 1 hour at room temperature, rinsed in TRIS buffer and processed for staining. Bound antibodies were visualized with DAB (3.3′-diaminobenzidine) (Sigma, Vienna, Austria) as substrate chromogen. Slides were counterstained with Hemalaun and coverslipped using Eukitt (Merck; Darmstadt, Germany). Brightfield microscopy with photography was performed using a Leica DMRB microscope and a Nikon Coolpix 995 camera.

A further protocol comprises the following steps:

Immunohistochemistry was performed on paraffin-embedded sections of HPV-16 positive biopsies derived from cervical carcinomas and control tissue specimens. The paraffin-embedded tissue specimens were sectioned at 2 and 5 μm. Sections were mounted on slides, deparaffinized in xylol (2×10 minutes), incubated for 5 minutes each in 100%, 90%, 80% and 70% ethanol and blocked in 5% H₂O₂ in absolute methanol for 15 minutes. Before immunostaining the sections were washed twice in TRIS buffer (7.75 g Tris-HCl pH 7.5, 8.78 g NaCl ad liter aqua dest/0.1% Tween 20) and processed for a 15 minutes blocking reaction in diluted (1:10 in TRIS buffer/1% BSA, 0.1% Tween 20) goat serum (DAKO, Hamburg, Germany). Sections were washed in TRIS/1% BSA/0.1% Tween 20 buffer and incubated with the first antibody (affinity purified polyclonal rabbit anti-HPV-16 E7 antibody) for 1 hour at room temperature in buffer B (10 μg/ml BSA/10 μg/ml NIH3T3 Lysate, 0.1 % Tween 20 in PBS). The samples were rinsed twice in TRIS/1% BSA/0,1% Tween 20 buffer and incubated for 1 hour at room temperature with the second antibody (Biotin-conjugated anti rabbit IgG, DAKO, Glostrup, Denmark). After the washing step in TRIS/1% BSA, ExtrAvidin-conjugated peroxidase solution (Amersham Biosciences, Vienna, Austria) was added and the samples were incubated for 1 hour at room temperature, rinsed in TRIS buffer and processed for staining. Bound antibodies were visualized with DAB (3.3′diaminobenzidine) (Sigma, Vienna, Austria) as substrate chromogen. Slides were counterstained with Hemalaun and coverslipped using Eukitt (Merck, Darmstadt, Germany). Brighffield microscopy with photography was performed using a Leica DMRB microscope and a Nikon Coolpix 995 camera (FIG. 4a-D).

### EXAMPLE 3.7.

### Immunohistochemical Detection of HPV-16 E7 in Prostate Derived Tissue

Biopsies were taken from 60 prostate carcinoma patients and samples from 60 patients were applied to an object slide together with negative controls. These slides are known to the expert as “tissue microarrays”. Tissue microarrays were stained with antibodies to HPV-16 E7 as described for cervical biopsies in Example 3.6. In this experiment, a subset of the carcinoma biopsies stained positive for HPV-16 E7, whereas other biopsies from different prostate cancer patients were staining negative. In these experiments, HPV-16 E7 was detected in roughly 10% of the prostate carcinoma specimens analyzed. This result suggests that the subset of the prostate carcinomas express high levels of HPV-16 E7 and thereby provide evidence for a role of HPV-16 E7 in prostate carcinoma (FIG. 5).

### EXAMPLE 3.8.

### Detection of HPV-16 E7 (Onco-)protein in Pre-neoplastic and Neoplastic Cells from Ectocervical Smears (PapSmear)

To determine the presence of HPV-16 E7 protein in ectocervical smears (PapSmear, routinely used for cervical cancer screening), superficial cells were obtained by cervical smear examination (PapSmear) from women with normal cervical squamous epithelia (healthy control) and cervical squamous intraepithelial lesions. Biopsies were taken at the department for Gynecology and Obstetrics at the University Hospital in Innsbruck/Austria. Biopsies were taken from nine cytologically normal patients and from twenty patients with abnormal cytological appearance (classified as Paplil by the physician). Superficial cells were obtained by cervical smear examination (PapSmear) from women with normal cervical squamous epithelia (healthy control) and cervical squamous intraepithelial lesions. Cells were streaked out on a glass slide and air dried. Subsequently, cells were fixed in 5% H₂O₂ (freshly dissolved in absolute methanol) for 15 minutes. Before immunostaining the sections were washed twice in TRIS buffer (7.75 g Tris-HCl pH 7.5, 8.78 g NaCl ad 1 liter aqua dest) and processed for a 15 minutes blocking reaction in diluted (1:10 in TRIS buffer/1% BSA) goat-serum (DAKO, Hamburg, Germany). Sections were washed in TRIS/1% BSA buffer and incubated with the first antibody (affinity purified polyclonal rabbit anti-HPV-16 E7 antibody described herein) for 1 hour at room temperature in buffer B (10 mg/ml BSA/10 mg/ml NIH3T3 lysate in PBS). The samples were rinsed twice in TRIS/1% BSA buffer and incubated for 1 hour at room temperature with the second antibody (Biotin-conjugated anti-rabbit IgG, DAKO, Glostrup, Denmark). After the washing step in TRIS/1% BSA, ExtrAvidin-conjugated peroxidase solution (Amersham Biosciences, Vienna, Austria) was added and the samples were incubated for 1 hour at room temperature, rinsed in TRIS buffer and processed for staining. Bound antibodies were visualized with DAB (3.3′-diaminobenzidine) (Sigma, Vienna, Austria) as substrate chromogen. Slides were counterstained with Hemalaun and coverslipped using Eukitt (Merck, Darmstadt, Germany). Brighffield microscopy with photography was performed using a Leica DMRB microscope and a Nikon Coolpix 995 camera.

Smears were stained by immunohistochemistry using the affinity-purified anti-HPV-16E7 antibody described herein. A representative example is shown in FIG. 6: The antibodies did not stain superficial cells in cervical smear from normal HPV-DNA negative ectocervix (FIG. 6A, patient ID SM28961), only normal basophile (grey) superficial cells with normal nucleus-cytoplasm relation are visible in this smear. In parallel, a ectocervical smear from a patient (patient ID WM20276), that had been classified as PapIIID and which was typed as HPV-16 DNA positive by PCR analysis, was analyzed by immunohistochemistry. A biopsy taken from the patient one day later revealed CINIII phenotype; later immunohistochemical analysis demonstrated high level expression of HPV-16 E7 in the tumor cells. Only a few normal basophile (grey/blue) superficial cells with normal nucleus-cytoplasm relation can be recognized. However, roughly 50% of the cells show enlarged nucleus-cytoplasm relation. These so-called koilocytes are stained by the E7 antibodies as indicated by the brown colour. Only these cells are stained by the E7 antibodies but not the normal squamous epithelial cells and columnar epithelium cells (usually contained in ectocervical smears). This demonstrates that the anti-HPV-16 E7 antibodies described herein provide a highly specific and sensitive marker for the detection of abnormal precursor malignant cells in cervical smear preparations (Pap Smears).

### EXAMPLE 3.9.

### Detection of HPV-16 E7 Protein in Pre-neoplastic and Neoplastic Cells from Ectocervical Smears-clinical Evaluation

According to Example 3.8, a further evaluation was carried out under local regulations in a blinded trial using patient material obtained at the University Hospital in Innsbruck/Austria. The evaluation was performed by experienced pathologists of the department of Gynecology and Obstetrics of the University Hospital Innsbruck/Austria who also validated the Pap smears and biopsies, respectively. For screening, Pap smears were taken from women with normal cervical squamous epithelia (healthy control) and cervical squamous intraepithelial lesions. Pap smears were collected with the consent of patients. In case of positive cytology, biopsies were taken at the department of Gynecology and Obstetrics at the University Hospital in Innsbruck/Austria.

From 19 women, two smears were taken: one for conservative examination (Papanicolao staining) and one for anti-HPV-16 E7 staining, using the affinity-purified anti-HPV-16 E7 antibody described herein, following the protocol described herein in example 3.8.

From the 19 Pap smear samples examined, 8 specimens had normal cytological appearance (classified as Pap II or lower, according to the Munich II classification (Soost H J. The Munich nomenclature; Recent Results Cancer Res 1993;133:105-11) and were tested by anti-HPV-16 E7 staining, using the affinity-purified anti-HPV-16 E7 antibody/serum of the invention.

In 11 specimens with abnormal cytological Pap smear appearance (classified either “higher than Pap II” or “Pap II, unclear”), HPV genotyping by PCR, conservative histomorphological examination of cervical tissue biopsy and anti-HPV-16 E7 staining, by using the affinity-purified rabbit anti-HPV-16 E7 antibody, was performed.

Excluding smears that were not assessable because of mucus or few cells, the results (Tab.1) show a clear correlation between an abnormal histology and the anti-HPV-16 E7 staining. As already demonstrated in Example 3.8, it can be shown in the present invention that anti-HPV-16 E7 antibodies described herein provide a highly specific and sensitive marker for the detection of abnormal precursor and malignant cells in Pap smears.

TABLE 2
Anti-HPV-16 E7 antibodies in Pap smears

| | HPV-Type | | | Anti-HPV-16 E7 |
|---|---|---|---|---|
| Nr | PCR | Pap class | Histology | staining |
| 1 | 16 | rezid. Pap IIID/IV | CIN III | + |
| 2 | ND | rezid. Pap IIID | CIN I | + |
| 3 | 16 | Pap II unclear | CIN III | +/−, few cells |
| 4 | ND | Pap II unclear | CIN III | + |
| 5 | 16 | Pap IIID | PE Carcinoma | + |
| 6 | ND | Pap IIID | CIN I | + |
| 7 | ND | rezid. Pap IIID | CIN I | + |
| 8 | 16 | Pap IV | CIN III | + |
| 9 | ND | Pap IIID | CIN I | + |
| 10 | 16 | Pap III | PE Carcinoma | few cells |
| 11 | 16 | rezid. Pap IIID | CIN II | + |
| 12 | ND | Pap II | ND | − |
| 13 | ND | Pap II | ND | − |
| 14 | ND | Pap II | ND | − |
| 15 | ND | Pap II | ND | − |
| 16 | ND | Pap II | ND | − |
| 17 | ND | Pap II | ND | − |
| 18 | ND | Pap II | ND | − |
| 19 | ND | Pap II | ND | − |

ND: not determined

### EXAMPLE 3.10.

### Detection of HPV-16 E7 in Tissue Homogenates by Sandwich ELISA

Control of HPV-16 E7Gene Expression in Biopsies by Westem Blot

The expression level of the HPV-16 E7 protein was studied in biopsy material derived from HPV-16 DNA positive cervical carcinoma patients and in histologically normal tissue specimens obtained from patients (HPV-DNA negative by PCR) who underwent hysterectomy for diseases unrelated to the cervix uteri. Three HPV-16 DNA positive and seven unrelated cervical biopsies were analysed in Western blot experiments using the affinity purified anti-HPV-16 E7 antibodies. The specimens derived from HPV-16 DNA positive cervical carcinoma patients were all positive, whereas in the unrelated tissues E7 was not detectable (FIG. 7). In one biopsy (# 2424) the E7 protein level was as high as in CaSki cells, a cell line derived from a HPV-16 positive cervical carcinoma (Schwarz (1985 Nature 314, 111-119)). In the other HPV-16-positive specimens the E7 level was lower; however, the different expression levels in the individual biopsies can be explained by the fact that the portion of tumor material in a given biopsy differs. No signal was obtained with cervical cancer biopsies derived from HPV-45 positive patients.

16E7-ELISA for Detection of HPV-16 E7 in Liquid Samples Derived from Cervical Biopsies

To establish detection of HPV-16 E7 in liquid samples, 96-well ELISA plates were coated with IgG fractions derived from the polyclonal antibody described in the invention. Coated plates were incubated with crude lysates derived from E. coli-expressing HPV-16 E7 and control E.coli lysates. This experiment was used to determine the effective threshold value for reliable detection of 16 E7 antigen. To this end, affinity-purified anti-HPV-16 E7 antibodies were conjugated with horseradish peroxidase. The conjugate was added to the plate and after four washing steps, 3,3′,5,5′-tetra-methylbenzidine (TMB; Boehringer Mannheim # 784 974) was added. After incubation for one hour, conversion of TMB was analyzed by densitometric analysis at 450 nm, using a Dynatec ELISA reader. The values obtained for E. coli lysate were plotted relative to protein concentration and used to determine the threshold value for the absorption. The background value plus three standard deviations were used to calculate the threshold value above which a sample as considered E7-positive. In the present experiment, the threshold was set to A₄₅₀>0.16. In a second step, tissue homogenates of the human biopsies described above were prepared, diluted in ELISA buffer and analyzed by 16E7 ELISA, as described above. Results are shown in appended Table 2.

TABLE 3
Comparative analysis of HPV-16 E7 expression in tissue biopsies
by 16E7 ELISA and Western blot

| | 16 E7-ELISA | | | | |
|---|---|---|---|---|---|
| Biopsy # | A₄₅₀ | pos/neg | 16E7 Western | HPV DNA | comment |
| 1839 | 0.08 | negative | − | negative | control |
| 1867 | 0.02 | negative | − | negative | control |
| 2413 | 0.55 | positive | ++ | HPV-16 | |
| 3358 | 0.06 | negative | − | negative | control |
| 3366 | 0.09 | negative | − | negative | control |
| 2227 | 0.04 | negative | − | HPV-45 | |
| 2257 | 0.08 | negative | − | HPV-45 | |
| 2295 | 0.32 | positive | + | HPV-16 | |
| 2424 | 1.4 | positive | +++ | HPV-16 | |
| 2622 | 0.03 | negative | − | negative | control |

Bioposies from five cervical carcinoma patients and biopsies from five hysterectomy patients without cervical neoplasia (control) were analyzed for their content of HPV-16 E7 both by ELISA and Western blot techniques. The table gives the absorption obtained in ELISA along with its evaluation (cutoff A₄₅₀>0.16) and results obtained by Western blot (see FIG. 7; grading derived from visual inspection). Also indicated DNA status of the patients, as determined by PCR analysis.

In the above described example 3.10, the following methods were employed

1. 16 E7-ELISA

Coating

Affinity-purified polyclonal antibodies from rabbits immunized with highly purified native HPV-16 E7 proteins as described in this invention were precipitated by addition of ammonium sulfate to a final concentration of 45%, followed by centrifugation. After three consecutive precipitations, the antibody was dissolved in water, dialyzed 3× against ice-cold PBS and used at a final concentration of 2 μg per ml to coat ELISA plates (Nunc, Vienna).

Conjugation

2 mg of affinity-purified antibodies according to the invention were conjugated with horseradish peroxidase. Briefly antibodies at 2 mg/ml in PBS (1:10 diluted) were dialyzed overnight at 4 C against sodium carbonate buffer (0.01 M NaHCO_{3/}Na₂CO₃, pH 9.3). POD (Sigma cat. # P6782) was dissolved at 8 mg/ml in water and incubated with 1/10 volume of 0.2M NalO₄ for 20 min at RT in the dark. Subsequently, the POD solution was dialyzed overnight at 4 C against 1 mM sodium acetate/pH 4.4.

For coupling, the POD solution was adjusted to pH 9.3 and immediately incubated with the antibody solution. To this end, 650 μl antibody solution was added to 215 μl POD solution. The mixture was incubated at RT for 2 h under gentle agitation in the dark. To stop the reaction, 43 μl of Na(BH₄) solution (4 mg/l aqua bidest.) was added and incubation continued for 2 h at RT. The conjugate was dialyzed against PBS overnight at 4 C, thiomersal was added to a final concentration of 0.1%. Conjugate was stored at 4 C.

Assay

After addition of TMB, peroxidase reaction and subsequent densitometric analyses in the ELISA reader were performed as described by the manufacturer.

2. E.coli Lysates

Construction of the Bacterial Expression Vector for HPV-16 E7

The HPV-16 E7 oncogene was amplified from the vector pX-HPV-16 E7 (Mannhardt (2000 Mol. Cell. Biol. 20, 6483-6495)) by PCR using Pfu DNA polymerase as Nde1/BamHI fragment. The sequence was inserted into the bacterial expression vector pET3a (Studier and Moffatt (1986 J. Mol. Biol. 189, 113-130)) prepared as Ndel/BamHI fragment generating the bacterial HPV-16 E7 expression vector pET3a-HPV-16 E7. The sequence encoding for HPV-16 E7 was verified by sequencing.

Preparation of Bacterial Lysates

The expression vector pET3a-HPV-16 E7/Clone 17 was transformed into E. coli strain BL21 (DE3) pLysS. The bacteria were grown to OD₆₀₀=0.5 and the expression of the E7 protein was induced for 3 hours at 37° C. by adding IPTG (Biomol, Hamburg, Germany) to a final concentration of 0.4 mM. For control lysates, the matemal strain E.coli (BL21 (DE3)pLysS) was used.

Bacteria were harvested by centrifugation for 10 minutes at 5000×g. The cell pellets were frozen in 20 ml ice-cold lysis buffer (50 mM KCl, 20 mM H₂KPO₄ [pH 7.8], 50 mM DTT, 5% glycerol, 1 μg/ml leupeptin, 1 mM PMSF, 1 mM NaF and 10 μg/ml Aprotinin) per liter bacterial culture. Cells were thawed on ice and lysed by sonication with glass beads (Sigma, Vienna, Austria) using a Sonifier 250 (Branson, Geneva, Switzerland). After centrifugation at 70 000×g for 1 hour, the supernatant was ammonium sulfate precipitated using 30% saturated (NH₄)₂SO₄ solution. The (NH₄) ₂SO4 pellet was dissolved in 150 mM Tris/Cl pH 7.8, 10 mM NaCl, dialysed against the same buffer and diluted to a final concentration of 10 mg/ml total protein.

3.Tissue Lysates

For protein extraction, biopsies were extracted in lysis buffer (10 mM Tris pH 7.5, 1% Triton X-100, 1 mM NAF, 0.2 mM PMSF). Samples are vortexed and redissolved by 20 strokes with a Branson sonifier on ice, followed by incubation on ice for 5 min. The sample is repeatedly frozen in liquid nitrogen, rethawed, and subsequently incubated on ice for another 15 minutes, followed by centrifugation for 30 min at 20.000 g. The supematant is directly used for Western blot analysis or 16E7 ELISA.

4. Western Blot (Immunoblot) Analysis

Cell extracts were separated on a 12.5% sodium dodecyl sulfate (SDS)-polyacrylamide gel, and proteins were transferred to a PVDF membrane (NEN, Boston, USA). The membrane was incubated in blocking buffer (0.05% Tween 20/5% low fat milk powder in PBS) for 1 hour at room temperature, washed in blocking buffer and incubated with the first antibody (affinity purified polyclonal rabbit anti-HPV-16 E7 antibody) for 1 hour at room temperature. After washing in blocking buffer, PBS/0.05% Tween 20 and PBS/5% low fat milk powder the membrane was incubated with the second antibody (peroxidase-conjugated anti-rabbit IgG, Promega, Mannheim, Germany) for 45 minutes at room temperature. The membrane was washed, and the bound antibodies were visualized by using the chemiluminescence Western blotting detection system (NEN, Boston, USA).

### EXAMPLE 3.11.

### Test of Crossreactivity of Affinity Purified Anti-HPV-16 E7 Antibodies of Rabbit with HPV-1, HPV-6, HPV-11, HPV-18, HPV-31, HPV-33, HPV-35, HPV-39, HPV-45, HPV-52, HPV-56, HPV-58 and HPV-59 E7 Protein by Indirect Immunofluorescence Analysis

U-2-OS cells were cultured in DMEM+10% FCS. For transient expression of cDNAs, cells were grown to about 80% confluence on glass coverslips coated with 0.05% gelatin. Transfections of 0.8 μg of expression vector pCMV-Tag2B containing the whole reading frame cDNAs of HPV-1, HPV-6, HPV-11, HPV-16, HPV-18, HPV-31, HPV-33, HPV-35, HPV-39, HPV-45, HPV-52, HPV-56, HPV-58 and HPV-59, respectively, fused with an N-terminal Flag-tag was performed by using Effectene (Qiagen, Hilden, Germany). Incubation was done in 5% CO₂ at 37° C. 24 h post-transfection, cells were prepared for indirect immunofluorescence according to standard protocols, including methanol fixation. After incubation with the primary antibody (affinity purified polyclonal rabbit anti-HPV-16 E7 antibody), and secondary antibody (FITC-conjugated anti-rabbit IgG, Jackson Immuno Research, Lot. 46138), cells were washed and embedded in Fluoromount G (Biozol, Eching, Germany). Samples were viewed by indirect immunofluorescence microscopy using the confocal scanning system MicroRadiance (Bio-Rad, Munich, Germany) in combination with a Zeiss Axiophot microscope. The following filters were used for FITC-derived fluorescence: excitation at 488 nm, emission at 515-530 nm. In these experiments, HPV-16 and HPV-18 E7 protein was detected in 5-10% of the transfected cells, whereas no signal was obtained in cells that have been transfected with the empty expression vector, or with expression vector expressing ubiquitin, HPV-1, 6, 11, 31, 33 , 35, 39, 45, 52, 56, 58 and 59 E7 protein (Tab. 3). This result clearly proves that the antibody is specific for the HPV-16 and HPV-18 E7 protein and also does not detect any non-specific background under these conditions. As an expression control the transfected cells also was tested by indirect immunofluorescence analysis through immuno detection of the fused flag epitope by an commercial available anti-flag antibody (primary antibody: mouse anti-Flag M2, Sigma, Lot. 102K9164) , secondary antibody: TRITC-conjugated anti-mouse, Jackson Immuno Research, Lot. 57187).

TABLE 4
Crossreactivity of affinity purified anti-HPV-16 E7 antibodies of rabbit
with HPV-1, HPV-6, HPV-11, HPV-18, HPV-31, HPV-33, HPV-35,
HPV-39, HPV-45, HPV-52, HPV-56, HPV-58 and HPV-59 E7 protein.

| | Expression | |
|---|---|---|
| | control by | Detection by |
| | western blot | immunofluorescence |
| HPV genotype | (anti-Flag) | (rabbit anti HPV-16 E7 ab) |
| HPV-1 | + | − |
| HPV-6 | + | − |
| HPV-11 | + | − |
| HPV-16 | + | + |
| HPV-18 | + | + |
| HPV-31 | + | − |
| HPV-33 | + | − |
| HPV-35 | + | − |
| HPV-39 | + | − |
| HPV-45 | + | − |
| HPV-52 | + | − |
| HPV-56 | + | − |
| HPV-58 | + | − |
| HPV-59 | + | − |
| Control 1: not transfected | − | − |
| cells | | |
| Control 2: expression vector | + | − |
| only with flag-tag | | |
| Control 3: Ubiquitin-flag | + | − |
| Control 4: HPV16 E7 without | − | + |
| an epitop tag | | |

### EXAMPLE 4

### Generation, Purification, Characterization and Quality Control of Polyclonal HPV-16 E7 Antibodies from Goat

### EXAMPLE 4.1.

### Generation of Polyclonal HPV-16 E7 Antibodies in Goat

Highly purified preparations of the HPV-16 E7 protein (see example 2) were used to produce highly specific polyclonal anti-HPV-16 E7 antibodies in goats (Saanen breed goat). Immunisation was done in parallels, employing the same protocol. (1^{st} injection) 1.1 ml complete Freund's adjuvant (Sigma, Vienna, Austria) was mixed with lmg HPV16-E7 protein in 1 ml gel-filtration buffer (see above). Mixing was performed manually by two syringes (one containing the adjuvant; one containing the antigen) connected via a Luer-lock mechanism (Oxf. Univ. Press; 2000: Practical approach series; ISBN 0-19-963711-3 Vol. Immunoassays; Edited by J. P. Gosling; p.28). To start with, the antigen solution was added into the adjuvant and for 5 min the mixture was passed rapidly back and forth between the syringes until a milky, thick emulsion was formed. For all further boost the described mixing method was employed except that incomplete Freund's adjuvant (IFA) was used instead of Complete Adjuvant (CFA)
1^{st} boost (2^{nd} injection): 28 days after immunisation; 1 mg HPV16-E7 in 1 ml gel filtration buffer+1.1 ml incomplete Freund's adjuvant.
2^{nd} boost (3^{rd} injection): 28 days after 1^{st} boost; 1 mg HPV16-E7 in 1 ml gel filtration buffer+1.1 ml incomplete Freund's adjuvant.
3^{rd} boost (4^{th} injection): 14 days after 2^{nd} boost; 1 mg HPV16-E7 in 1 ml gel filtration buffer+1.1 ml incomplete Freund's adjuvant.
1^{st} production bleeding: 14 day after the 3^{rd} boost: 400ml of blood was taken from one jugular vein.
4^{th} boost (5^{th} injection): 14 days after 1^{st} production bleeding; 1 mg HPV16-E7 in 1 ml gel filtration buffer+1.1 ml incomplete Freund's adjuvant.
5^{th} boost (6^{th} injection): 14 days after 4^{th} boost; 1 mg HPV16-E7 in 1 ml gel filtration buffer+1.1 ml incomplete Freund's adjuvant.
2^{nd} production bleeding: 14 day after the 5^{th} boost; 550 ml of blood was taken from one jugular vein.
6^{th} boost (7^{th} injection): 14 days after 2^{nd} production bleeding; 1 mg HPV16-E7 in 1 ml gel filtration buffer+1.1 ml incomplete Freund's adjuvant.
7^{th} boost (8^{th} injection): 14 days after 4^{th} boost; 1 mg HPV16-E7 in 1 ml gel filtration buffer+1.1 ml incomplete Freund's adjuvant.
3^{rd} production bleeding: 14 day after the 7^{th} boost; 700 ml of blood was taken from one jugular vein. Tab.3 shows the applied immunisation—boosting—bleeding time table.

Further boost were administered and bleedings were taken following the same time schedule as described above. The blood volume taken at all further bleedings was 700 ml, giving approximately 350 ml of antiserum. Two day before each production bleeding, a small volume of blood was taken in addition to investigate antibody quality and titer by westem-blotting (E7/2 cell-lysate;125 pg protein/lane) and ELISA (1 μg purified HPV16-E7/well).

TABLE 5
timetable to generate polyclonal, anti HPV16-E7 antibodies in goat.

| day | application of HPV16-E7 | bleedings |
|---|---|---|
| 0 | immunisation: 1 mg HPV16-E7 | pre-immune serum |
| (days | in complete Freund's adjuvant | |
| since | | |
| previouse | | |
| event) | | |
| 28 | 1^{st} boost: 1 mg HPV16-E7 | |
| (+28) | in incomplete Freund's adjuvant | |
| 56 | 2^{nd} boost: 1 mg HPV16-E7 | 1^{st} test bleeding |
| (+28) | in incomplete Freund's adjuvant | |
| 70 | 3^{rd} boost: 1 mg HPV16-E7 | 2^{nd} test bleeding |
| (+14) | in incomplete Freund's adjuvant | |
| 82 | | 3^{rd} test bleeding |
| (+12) | | |
| 84 | | 1^{st} production bleeding |
| (+2) | | 400 ml blood = 200 ml |
| | | serum |
| 98 | 4^{th} boost: 1 mg HPV16-E7 | |
| (+14) | incomplete Freund's adjuvant | |
| 112 | 5^{th} boost: 1 mg HPV16-E7 | |
| (+14) | in incomplete Freund's adjuvant | |
| 124 | | 4^{th} test bleeding |
| (+12) | | |
| 126 | | 2^{nd} production bleeding |
| (+2) | | 550 ml blood = 275 ml |
| | | serum |
| 140 | 6^{th} boost: 1 mg HPV16-E7 | |
| (+14) | in incomplete Freund's adjuvant | |
| 154 | 7^{th} boost: 1 mg HPV16-E7 | |
| (+14) | in incomplete Freund's adjuvant | |
| 166 | | 5^{th} test bleeding |
| (+12) | | |
| 168 | | 3^{rd} production bleeding |
| (+2) | | 700 ml blood = 300 ml |
| | | serum |

### EXAMPLE 4.2.

### Quality Control of Polyclonal Goat Anti HPV16-E7 Anti Serum by Western Blotting

To investigate the quality of the obtained antisera (test-bleedings and production bleedings), cell extracts of NIH-3T3 fibroblast (negative control) and E7/2 cells (i.e. NIH 3T3 fibroblast stably transfected with HVP16-E7) were separated on a 12.5% sodium dodecyl sulfate (SDS)-polyacryl-amide gel, and proteins were transferred to a PVDF membrane (NEN, Boston, USA). The membrane was washed 2×15 min in PBS and incubated in blocking buffer (PBS, 0.05% Tween 20, 5% low fat milk powder) at 4° C. over night. After blocking, the membrane was cut into stripes (always containing one lane of NIH-3T3 and one lane of E7/2 lysate) and incubated for 1 hour at room temperature with goat serum diluted 1:400 in blocking buffer. Upon washing (15 min blocking buffer; 15 min PBS, 0.05% Tween 20; 2×5 min in PBS, 5% low fat milk powder), the PVDF stripes were incubated with the second antibody (peroxidase-conjugated anti-goat IgG, Promega, Mannheim, Germany) 1:10000 in blocking buffer for 1 hour at room temperature. The stripes were washed as stated above but with two additional washing steps (2×5 min in PBS) and the bound antibodies were visualized by using the ECL+Chemiluminescence Western blotting detection system (NEN, Boston, USA) (FIG. 10)

FIG. 10 shows a Western blot to investigate immune-response and quality of generated goat anti-sera. After the transfer of protein to PVDF membrane, membrane was cut into 7 stripes. Each of the 7 stripes contained 125 μg of NIH-3T3 cell lysate (left lane) and 125 μg of E7/2 lysate (right lane). Incubation of the PVDF-stripes was as follows: stripe 1—pre-immune-serum; stripe 2—2^{nd} test bleeding; stripe 3—3^{rd} test bleeding; stripe 4—1^{st} production bleeding; stripe 5—2^{nd} production bleeding; stripe 6—3^{rd} production bleeding; stripe 7—4^{th} production bleeding.

### EXAMPLE 4.3.

### Quality Control of Polyclonal Goat Anti HPV16-E7 Anti Serum by Titer Determination in ELISA

To determine the titer of all obtained anti-sera, a direct ELISA was developed as follows:
Coating: Lanes 2-12 of a Nunc Maxisorb 96-well plate (Nalge, Belgium) were coated with 1 μg purified HPV16-E7 protein (Example 2) in 100 μl coating buffer (100 mM NaHCO₃, pH 9.6)/well at 4° C. over night (A3-H12). Two lane (1,2) were coated with coating-buffer only as zero control.
Washing: 3×300 μl washing-buffer (PBS, 5 mM EDTA, 0.05% (v/v) Tween 20; pH 7.4);
Blocking: all wells were filled with blocking-buffer (PBS, 1% (w/v) BSA, 5 mM EDTA, 0.05% (v/v) Tween 20; pH 7.4) and kept for 2 hours at room temperature.
Washing: 3×300 μl washing-buffer (PBS, 5 mM EDTA, 0.05% (v/v) Tween 20; pH 7.4);
Addition of serum-samples: pre-immune serum and anti-sera, diluted in washing-buffer were added to the wells in duplicates; 100 μl/well was added—dilutions were 1:1000, 1:5000, 1:25000, 1:50000 and 1:100000. Incubation time was 1 hour at room temperature;
Washing: 3×300 μl washing-buffer (PBS, 5 mM EDTA, 0.05% (v/v) Tween 20; pH 7.4);
Addition of 2^{nd} antibody: 100 μl of HRP-labelled rabbit anti goat-IgG (DAKO) in a 1:20000 dilution was added per well; as diluent washing buffer was used. Incubation time with labelled anti-antibodies was 1 hour at room temperature;
Washing: 3×300 μl washing-buffer (PBS, 5 mM EDTA, 0.05% (v/v) Tween 20; pH 7.4);
Detection: Prior to detection, a HRP-substrate solution was prepared by adding 32 mg of ABTS powder (2,2-Azino-bis(3-ethylbenzthiazoline-6-sulfonic acid); Sigma, Steinheim Germany; A-1888;) to 100ml of ABTS-buffer (3.379 mM Natriumperborat-3-Hydrat (Merck 106560); 4.187 mM Zitronensaeure-1-Hydrat (Merck 100244); 63 mM Dinatriumhydrogenphosphat-2-Hydrat (Merck 106580); adjusted to pH 4.4 with HCl). Substrate solution (100 μl) was added to each well and the plate was incubated at 37° C. in the dark. Colour-development was measured after 30 min at 405 nm in a BenchmarkTM Microplate Reader (Bio-Rad). Evaluation was performed by MS-Excel . A serum with a titer greater than 5.000 was assumed as high-quantity and as such suitable for antibody-purification.
Result: FIG. 11 Serum samples from test bleedings (tb) and production bleedings (pb) obtained from one of two goats, immunised with HPV16-E7 according to the time schedule given in Tab. 5 were used. The panel show the calculated result of the titer-ELISA after 30 min of colour development. Signals from 96-well plate lane 1/2 were subtracted (background control) all samples were measured in duplicates, S.D. is shown. The asterisks indicate the number of anti-gene administrations between the bleedings. In all bleedings tested, the observed titer was above 100000.

### EXAMPLE 4.4

### Purification of Polyclonal Goat-anti-HPV16-E7 Antibodies by Affinity Chromatography

Two columns were used to purify polyclonal HPV16-E7 antibodies from animal serum by affinity chromatography.

Column 1: (column to purify total IgG from antiserum). A 5 ml HiTrap Protein G HP column (Amersham Biosciences, Vienna, Austria) was used according to the manufacturers protocol to isolate total IgG from antiserum.

Column 2: As affinity matrix CNBr-activated Sepharose 4B (Amersham Biosciences, Vienna, Austria) was used. Prior to coupling, the ligand (HPV16-E7) was dialysed (from 150 mM Tris/HCl, 150 mM NaCl, 10 mM DTT, pH 7.8) into coupling buffer (100 mM NaHCO₃, 500 mM NaCl, pH 8.3) as Tris would interfere with the coupling reaction. 1.7 g of the freeze dried affinity matrix were activated according to the manufacturers protocol and transferred into coupling buffer containing 7 mg of HPV16-E7 protein (determined according to Bradford, using BSA as standard). Coupling was performed for 2 hours at room temperature in a 50 ml Falcon tube attached to a rotating platform. Once coupling was completed, the affinity matrix was packed into a XK16 FPLC column (Amersham Biosciences, Vienna, Austria) by gravity. The settled gel-bed (6 ml) was then washed with 5 column volumes of coupling buffer and 5 column volumes of blocking buffer (1M Ethanolamine, pH 8.0). The column was then left at room temperature for 2 hours with out agitation to block remaining active groups, and thereafter washed with 5 column volumes high pH buffer (100 mM Tris/HCl, 500 mM NaCl, pH 8.0) and 5 column volumes low pH buffer (100 mM Na-acetate, 500 mM NaCl, pH 4.0). The cycle high pH-wash/low pH-wash was repeated 5 times. Finally the column was attached to the FPLC system and equilibrated to running buffer (PBS, 200 mM NaCl, 5 mM EDTA, 0.05% NaN3, pH 7.4). Protein contents (measured according to Bradford) of coupling buffer before and after coupling, and of all through-runs and wash buffers collected, revealed a coupling efficiency of approximately 85% of HPV16-E7 protein to the column. The ligand density was 1 mg/ml gel-bed; the column volume was 6 ml.

Goat-antiserum (10 ml) was diluted in 20 ml running buffer (PBS, 200 mM NaCl, 5 mM EDTA, 0.05% NaN₃, pH 7.4), filtered trough a 0.45 μm sterile filter and passed over a 5 ml Protein G column using an Äkta Prime system (Amersham Biosciences) at a flow rate of 5 ml/min. The column was extensively washed with running buffer until the baseline was zero (5 ml/min). Total IgG was eluted in 1 ml fractions (1ml/min) with 100 mM Glycine, 0.05% NaN₃, pH 2.5 into 1.5 ml reaction vials containing 200 μl of 1 M KH₂PO₄/K₂HPO₄-buffer pH 7.4 to neutralize the low pH of the elution buffer. Material was then passed over the affinity column (equilibrated in 5 CV running buffer at 10 ml/min) at a flow rate of 10 ml/min in a closed circle for 30 min until an equilibrium was reached. The column was then washed with 10 CV PBS, 1 M NaCl, 5 mM EDTA, 0.05% NaN₃, pH 7.4 at 10 ml/min until the baseline reached zero. After re-equilibration into running buffer (10 column volumes at 10 ml/min), polyclonal anti HPV16-E7 antibodies were eluted (1 ml/min) with 100 mM Glycine, 0.05% NaN₃, pH 2.5 into 1.5 ml reaction vials containing 200 μl of 1 M KH₂PO₄/K₂HPO₄ buffer to neutralize the low pH of the elution buffer (fraction size was 1 ml). After elution, the column pH was set back to neutral by passing 10 CV of 3 M KSCN, 150 mM KCl, 10 mM KH₂PO₄/K₂HPO₄, pH 7.4 and 20 column volumes of running buffer through the system.

Anti HPV16-E7 antibody containing fractions were tested further by western-blotting using NIH 3T3- and E7/2-cell lysat as stated above. Blots were probed with anti HPV16-E7 antibody containing fractions, as second antibody, commercially available HRP-labeled anti goat IgG was used. Clean antibody fractions (i.e. factions giving a clean signal, even after film-exposure for more then prolonged 15 min) were pooled and adjusted to 0.5 mg/ml (goat 1) and 0.25 mg/ml (goat 2) respectively in 166 mM KH₂PO₄/K₂HPO₄, 83 mM Glycine pH 7.0.

### EXAMPLE 4.5.

### Conservation and Stabilisation of Affinity Purified Polyclonal Goat Anti HPV16-E7 Antibodies by Lyophilisation

Aliquots of 500 μl each were lyophilised over night and transferred to 4° C. Lyophilised antibodies were kept in sealed reaction vials, stored in a container containing silica-gel to avoid exposure to humidity. For reconstitution 0.05% NaN₃ aquarious solution was used to avoid bacterial growth during prolonged storage in liquid state.

Western-blots was done to prove the sensitivity of the reconstituted, lyophilized polyclonal goat anti HPV16-E7 antibodies.

Lyophilised antibodies were kept for at least 2 month at 4° C. To prove the sensitivity of reconstituted lyophilised material in comparison to material which had not been lyophilised but stored for the same period of time at 4° C., lyophilised antibodies were re-hydrated in 0.05% NaN₃ in steril water to the initial concentration. NIH-3T3 mouse fibroblast (negative control) and E7/2 cells (i.e. NIH 3T3 mouse fibroblast stably transfected with HVP16-E7) were separated, blotted as stated above, and probed with purified anti HPV-16 antibodies from 2 goats, both subjected and not-subjected to lyophilisation respectively. Antibodies from goat 1 and goat 2 were used in a 1:400 and 1:800 dilution respectively (FIG. 12).

As no loss of antibody-activity due to the lyophilisation process was observed, in a next step it was investigated by Western blotting, whether the purified, reconstituted antibody would recognise HPV16-E7 protein in cells with low-level expression of the antigen. Again 125 μg of cell-lysate derived from C33A cells (human cervix-carcinoma cells, which do not contain HPV16-E7 protein ; negative control), Caski- and SIHA-cells (human cervix carcinoma cell with different expression levels of HPV 16-E7), NIH 3T3 and E7/2 cells were separated on a 12.5% sodium dodecyl sulfate (SDS)-polyacryl-amide gel, and transferred to a PVDF membrane (NEN, Boston, USA). The membrane was treated a stated above and probed with reconstituted antibodies from goat 1 and goat 2 in a 1:400 and 1:800 dilution respectively. After washing membranes were incubated with the second antibody (peroxidase-conjugated anti-goat IgG, Promega, Mannheim, Germany) 1:10000 in blocking buffer and washed again. Bound antibodies were visualized by using the ECL+Chemiluminescence Western blotting detection system (NEN, Boston, USA). (FIG. 2c.). Both antibodies used (goat 1 and 2) were capable of recognizing HPV16-E7 in SIHA, Caski and E7/2 cell-lysate without any unspecific background staining. Negative controls (C33A and NIH 3T3) gave no signal at all; FIG. 2c.

Result: FIG. 12 shows a Western-blots as quality control for purified, reconstituted anti HPV16-E7 antibody from two goats. Membranes were cut into stripes for antibody exposure, in the way that each stripe contained control and test lanes. Strips containing cell-lysate from NIH-3T3 fibroblast (control) and E7/2 cells (test) were probed with lyophilised (1) and not-lyophilised (2) antibodies from goat 1 and lyophilised (3) and not-lyophilised (4) antibodies from goat 2. No difference in signal-strength or loss of specificity before and after lyophilisation was observed.

### EXAMPLE 4.6.

### Labeling of Reconstituted, Lyophilized Polyclonal Goat Anti HPV16-E7 Antibodies for Use in Direct ELISAS

Purified anti HPV16-E7 antibodies from goat were labeled with an AP-labeling kit from Roche (Roche Diagnostics, Mannheim Germany). Lyophilized antibodies from goat 2 (16 aliquots) were reconstituted in 4 ml conjugation buffer, concentrated to 180 μl on a Vivaspin-concentrator (VivaScience, Hanover, Germany) cut-off 10 kDa and dialyzed over night at 4° C. against 100 ml conjugation buffer. Final volume was 180 μl of conjugation buffer, containing 6.93 mg/ml IgG (i.e. 1.24 mg IgG, suitable for 4 labelling reactions). Labelling was performed according to the manufacturers protocol, giving 20 aliquots of 50 μl labelled anti-bodies. Aliquots were shock frozen in liquid N₂ and stored at −80° C. until further use. To establish the working concentration of the labelled antibody, a direct ELISA was set up as follows:
Purified HPV16-E7 protein was diluted in coating buffer (100 mM NaHCO₃; pH9.6) to give a stock solution of 10 μg/ml. In a further series, 10-fold dilutions were prepared to give 1 μg, 100 ng, 10 ng, 1 ng, 100 pg, 10 pg/ml coating puffer; the zero-control was coating buffer only.

To immobilise HPV16-E7 a Maxisorb-96-well plate (Nalge Nunc; Ridderstraat; Belgium) was coated with 100 μl of solution/well over night at 4° C.

Coating: row A: coating-buffer only; row B: 1 pg/well; row C: 10 pg/well; row D: 100 pg/well; row E: 1 ng/well; row F: 10 ng/well; row G: 100 ng/well; row H: 1 μg/well;

Next day:

Washing: 3×300 μl washing-buffer (PBS, 5 mM EDTA, 0.05% (v/v) Tween 20; pH 7.4);

Blocking: 300 μl PBS, 0.05% (v/v) Tween 20, 5 mM EDTA, 1% (w/v) BSA, pH 7.4; 2 hours at room temperature;

Washing: 3×300 μl washing-buffer (PBS, 5 mM EDTA, 0.05% (v/v) Tween 20; pH 7.4);

Addition of AP-labelled antibody: 100 μl/well diluted in washing buffer. Column 1: washing buffer only (zero control); column 2: antibody 1:500; columns 3+4: antibody 1:1000; columns 5+6: antibody 1:5000; columns 7+8: antibody 1:10000; columns 9+10: antibody 1:20000; columns 11+12: antibody 1:30000; incubation time was 1 hour at room temperature.

Washing: 3×300 μl washing-buffer (PBS, 5 mM EDTA, 0.05% (v/v) Tween 20; pH 7.4);

Detection: 100 μl/well substrate solution (Sigma FaSt™ p-Nitrophenyl Phosphate Tablets; Sigma, Steinheim Germany; N-2770); Colour development was measured after 30 min at 37° C. in a Benchmark Microplate Reader (Bio-Rad) at 405 nm.

Result: FIG. 3d.) Best detection of immobilized HPV16-E7 was achieved by using a 1:5000 dilution of the labeled antibody, as dilutions of 1:500 and 1:1000 reach saturation and dilutions 1:10000-1:30000 where not sensitive enough.

### EXAMPLE 4.7

### Direct Sandwich ELISA to Detect HPV-16 E7 Protein; Proof of Concept

A direct sandwich ELISA with unlabeled goat anti-HPV-16 E7 antibody as capture antibody and with AP-labeled goat anti-HPV16-E7 antibody as detection antibody was set up.

Samples used to determine the sensitivity and reproducibility of the developed ELISA included
i.) serum of a healthy donor (control) in a 2-fold dilution 1:50 to 1:3200 in washing-buffer (PBS, 5 mM EDTA, 0.05% (v/v) Tween 20; pH 7.4);
ii.) serum of a healthy donor containing purified HPV-16 E7 (stock-solution 1 μg HPV-16 E7/ml) in 2-fold dilutions 1:50 to 1:3200 in washing-buffer (PBS, 5 mM EDTA, 0.05% (v/v) Tween 20; pH 7.4);
iii) washing-buffer containing purified HPV16-E7 (stock-solutions 1 μg HPV16-E7/ml) in 2-fold dilutions 1:50 to 1:3200 in washing-buffer (PBS, 5 mM EDTA, 0.05% (v/v) Tween 20; pH 7.4);

Coating: Purified, unlabelled goat anti-HPV16-E7 antibody was diluted in coating buffer (100 mM NaHCO₃; pH9.6) to a final concentration of 2.5 μl/ml. To immobilise the capture antibody, a Maxisorb-96-well plate (Nalge Nunc; Ridderstraat; Belgium) was coated with 100 μl of that solution at 4° C. over night to achieve a coating density of 250 ng antibody/well.

Washing: 3×300 μl washing-buffer (PBS, 5 mM EDTA, 0.05% (v/v) Tween 20; pH 7.4);

Blocking: 300 μl PBS, 0.05% (v/v)Tween 20, 5 mM EDTA, 1% (w/v) BSA, pH 7.4; 2 hours at room temperature;

Washing: 3×300 μl washing-buffer (PBS, 5 mM EDTA, 0.05% (v/v) Tween 20; pH 7.4);

Addition of samples: Samples were applied in duplicates; 100 μl per well; incubation time was 1 hour at room temperature.

Washing: 3×300 μl washing-buffer (PBS, 5 mM EDTA, 0.05% (v/v) Tween 20; pH 7.4);

AP-labelled antibody: 100 μl of AP-labelled goat anti-HPV16-E7 antibody, diluted 1:5000 in washing-buffer; was added per well; incubation time was 1 hour at room temperature.

Washing: 3×300 μl washing-buffer (PBS, 5 mM EDTA, 0.05% (v/v) Tween 20; pH 7.4);

Detection: 100 μl/well substrate solution (Sigma FaSt™ p-Nitrophenyl Phosphate Tablets; Sigma, Steinheim Germany; N-2770); Colour development was measured after 30 min at 37° C. in a Benchmark Microplate Reader (Bio-Rad) at 405 nm.

Result: FIG. 13) The ELISA evaluation of the signals was obtained after 30 min at 37° C. In the control serum of the healthy donor, no specific HPV16 E7 signal was detected (even after prolonged developing time of 1.5 hours). Signals observed were regarded as back-ground. In serum and buffer containing HPV16 E7 protein respectively, a specific signal was observed down to a dilution of 1:1600-1:3200, corresponding to an antigen concentration of 62.5 pg -31.25 pg/well. As expected, in the “test-serum” serum-components interfered with the added HPV16 E7 protein, thereby slightly suppressing the detection signals.

### EXAMPLE 4.8

### Direct Sandwich ELISA to Detect HPV16-E7 Protein in Serum Samples from Cervix-carcinoma Patient

Using the above described set-up, serum specimens of two healthy donors and of a cervix-carcinoma patient, known to by HPV16 positive by PCR-analysis, were tested for the presence of HPV16-E7 protein. All ELISA steps were performed as stated above. Dilutions of all serum samples were 1:0, 1:2, 1:4 and 1:8 in washing-buffer (PBS, 5 mM EDTA, 0.05% (v/v) Tween 20; pH 7.4).

Result: FIG. 14.) The ELISA evaluation of the signals was obtained after 30 min at 37° C. In the two control sera of the healthy donors, no specific HPV16 E7 signal was detected. Signals observed were regarded as back-ground (back-ground signal from control-serum). In the patient serum HPV16-E7 protein could be detected throughout all dilutions).

### EXAMPLE 4.9.

### Test of Crossreactivity of Affinity Purified Anti-HPV-16 E7 Antibodies of Goat with HPV-1, HPV-6, HPV-11, HPV-18, HPV-31, HPV-33, HPV-35, HPV-39, HPV-45, HPV-52, HPV-56, HPV-58 and HPV-59 E7 Protein by Indirect Immunofluorescence Analysis

According to example 3.5., indirect immunofluorescence analysis was done in U-2-OS cells cultured in DMEM+10% FCS. For transient expression of HPV E7 cDNAs, cells were grown to about 80% confluence on glass coverslips coated with 0.05% gelatin. Transfections of 0.8 μg of expression vector pCMV-Tag2B (Stratagene) including the whole reading frame cDNAs of HPV-1, HPV-6, HPV-11, HPV-16, HPV-18, HPV-31, HPV-33, HPV-35, HPV-39, HPV-45, HPV-52, HPV-56, HPV-58 and HPV-59 fused with an N-terminal Flag-epitop-tag was performed by using Effectene (Qiagen, Hilden, Germany). Incubation was done in 5% CO₂ at 37° C. 24 h post-transfection, cells were prepared for indirect immunofluorescence according to standard protocols, including methanol fixation. After incubation with the primary antibody (affinity purified polyclonal goat anti-HPV-16 E7 antibody), and secondary antibody (FITC-conjugated anti-goat IgG, Jackson Immuno Research, Lot. 46138), cells were washed and embedded in Fluoromount G (Biozol, Eching, Germany). Samples were viewed by indirect immunofluorescence microscopy using the confocal scanning system MicroRadiance (Bio-Rad, Munich, Germany) in combination with a Zeiss Axiophot microscope. The following filters were used for FITC-derived fluorescence: excitation at 488 nm, emission at 515-530 nm. In these experiments E7 protein of HPV-16, HPV-31, HPV-33, HPV-35, HPV-39, HPV-45, HPV-52, HPV-56, HPV-58 and HPV 59 E7 was detected in 5-10% of the transfected cells, whereas no signal was obtained in cells that have been transfected with the empty expression vector or with expression vector expressing ubiquitin or HPV-11 E7 protein. This result clearly demonstrates that the antibody binds highly specific to HPV-16, HPV-31, HPV-33, HPV-35, HPV-39, HPV-45, HPV-52, HPV-56, HPV-58 and HPV 59 E7 protein and also does not detect any non-specific background under these conditions (Tab. 4). As an expression control the transfected cells also were tested by indirect immunofluorescence analysis (TRITC-conjugated anti-mouse, Jackson Immuno Research, Lot. 57187) through detection of the fused flag epitope by an commercial available anti-flag antibody (primary antibody: mouse anti-Flag M2, Sigma, Lot. 102K9164), secondary antibody: TRITC-conjugated anti-mouse, Jackson Immuno Research, Lot. 57187).

TABLE 6
Crossreactivity of affinity purified anti-HPV-16 E7 antibodies of goat with
HPV-1, HPV-6, HPV-11, HPV-18, HPV-31, HPV-33, HPV-35, HPV-39,
HPV-45, HPV-52, HPV-56, HPV-58 and HPV-59 E7 protein.

| | Expression | |
|---|---|---|
| | control by | |
| | western blot | Detection by |
| | (anti-Flag- | immunofluorescence |
| HPV genotype | ab) | (goat anti-HPV-16 E7 ab) |
| HPV-1 | + | − |
| HPV-6 | + | − |
| HPV-11 | + | − |
| HPV-16 | + | + |
| HPV-18 | + | − |
| HPV-31 | + | + |
| HPV-33 | + | + |
| HPV-35 | + | + |
| HPV-39 | + | + |
| HPV-45 | + | + |
| HPV-52 | + | + |
| HPV-56 | + | + |
| HPV-58 | + | + |
| HPV-59 | + | + |
| Control 1: not transfected | − | − |
| cells | | |
| Control 2: expression vector | + | − |
| only with flag-tag | | |
| Control 3: Ubiquitin-flag | + | − |
| Control 4: HPV16 E7 without | − | + |
| an epitop tag | | |

### Example 4.10.

### Competition Experiments with Anti HPV16-E7 pAB of the Present Invention

To verify the specificity of anti HPV16-E7 pAB described herein in IF, additional blocking experiment with GST4usion proteins were performed. Three GST-fusion-proteins were generated and attached to a bead-matrix. Proteins were i.) GST as control, ii.) HPV11 -E7-GST (full length) and iii.) HPV16-E7-GST (full lengh).

Affinity purified anti HPV16-E7 goat pABs were then adsorbed against a known concentration of GST-proteins, still attached to the beads, in a batch-wise procedure (0.0125 mg/ml antibodies per 0.1 mg GST-fusion protein). Pre-adsorbed antibodies were then tested in IF-experiments according to example 3.5. (indirect immunofluorescence analysis) on U2Os cells expressing HPV16-E7 protein.

Results are shown in FIG. 16. Panel A: No diminished signal was found after adsorption of antibodies against to GST- and GST-HPV11-E7-protein. The signal was completely blocked after adsorption to HPV16-E7-protein.

### EXAMPLE 4.11.

### Detection of HPV-16, 31, 33/58 E7 Protein in Pre-neoplastic and Neoplastic Cells from Ectocervical Smears by Goat Anti-HPV-16 E7 Antibodies

To test crossreactivity of goat anti-HPV-16 E7 antibodies in patients material, a further evaluation was carried out according to Example 3.9 under local regulations in a blinded trial using patient material obtained at the University Hospital in Innsbruck/Austria. The evaluation was performed by experienced pathologists of the department of Gynecology and Obstetrics of the University Hospital Innsbruck/Austria who also validated the Pap smears and biopsies, respectively. For screening, Pap smears were taken from women diagnosed with cervical squamous intraepithelial lesions. Pap smears were collected with the consent of patients. In case of repetitive positive cytology, biopsies were taken at the department of Gynecology and Obstetrics at the University Hospital in Innsbruck/Austria.

From 16 women with an abnormal diagnosis, two smears were taken: one for conservative examination (Papanicolao staining) and one for anti-HPV E7 staining, using the affinity-purified goat anti-HPV-16 E7 antibody described herein, following the protocol described herein in example 3.8.

In all specimens with abnormal cytological Pap smear appearance (classified higher than Pap II), HPV genotyping by PCR, conservative histomorphological examination of cervical tissue biopsy and anti-HPV E7 staining, by using the affinity-purified goat anti-HPV-16 E7 antibody, was performed.

Excluding smears that were not assessable because of mucus or few cells (7, not shown), the results (Tab. 5) demonstrate the high specificity of goat anti-H PV-16 E7 antibodies to E7 proteins of high risk HPV genotypes 16, 31, 33/58.

TABLE 7
Crossreactivity of anti-HPV-16 E7 antibodies of goat in Pap smears

| | HPV-Type | | | Anti-HPV-16 E7 |
|---|---|---|---|---|
| Nr | PCR | Pap class | Histology | staining |
| 1 | 16 | rezid. Pap IIID/IV | CIN III | + |
| 2 | 33, 58 | rezid. Pap IIID | CIN I | + |
| 3 | 16 | Pap IIID | PE Carcinoma | + |
| 4 | 33, 58 | Pap III | CIN III | + |
| 5 | 33, 58 | rezid. Pap III | CIN II | + |
| 6 | 16 | Pap IV | CIN III | + |
| 7 | 16 | rezid. Pap IIID | CIN II | + |
| 8 | 31 | Pap IIID | CIN II | + |
| 9 | 31 | Pap IV | CIN II | + |

### EXAMPLE 5. Recombinant Expression of HPV-18 E7 Protein and Generation of Polyclonal Antibodies Against HPV-18E7 Protein in Rabbit, Goat and Mice

5.1. Construction of the Bacterial Expression Vector for HPV18-E7

Material and method was as stated in example 1 (Construction of the bacterial expression vector for HPV16-E7). The insert encoding for HPV18-E7 protein was amplified from genomic DNA extracted from patients material. The sequence was inserted into the bacterial expression vector pET3a via NdeI/BamHI restriction sides, generating the bacterial HPV18-E7 expression vector pet3a-HPV18-E7/clone 5C. The sequence encoding for HPV18-E7 was verified by sequencing.

5.2. Expression and Purification of Recombinant HPV18-E7 Protein

Material and method was as stated in example 2 (Expression and purification of recombinant HPV16-E7 protein) with slight modifications:
i.) The over night culture was grown at room temperature; the medium was LB, 100 μg/ml Ampicillin, 25 μg/ml Chloramphenicol and 2% Glucose. End O.D.₆₀₀of the over night culture was 0.8-1.0.
ii.) For protein expression, NZCYM medium (100 μg/ml Ampicillin; +25 μg/ml Chloramphenicol; no glucose) was inoculated with 1-2% of the overnight culture and grown to an O.D.₆₀₀ of 0.3 at 37° C. Thereafter the culture was cooled to 16° C. and as an O.D.₆₀₀ had reached 0.5, IPTG was added to a final concentration of 0.4 mM ; bacteria were harvested 6 hours after induction; expression temperature was 16° C.; end O.D.₆₀₀ of the culture was 1.0-1.2;

The purification of the HPV18-E7 protein was as stated in example 2. FIG. 17 shows samples taken during a representative small-scale HPV18-E7 purification. Examples from ion-exchange chromatography (IEC; panel A) and gelfiltration (GF; panel B) are shown. For characterization of the purified HPV18-E7 protein see “Characterization of antigens used for immunization”. Purified HPV18-E7 protein shows 2 characteristic bands in SDS-PAGE under reducing conditions as depicted in FIG. 17 panel C.

5.3. Characterization of Antigen Used for Immunization

Purified 18-E7 protein that was used for the immunisation of goats, rabbits and mice was characterize by Eurosequence b.v. (Meditech Center L.J. Zielstraweg 1, 9713 GX Groningen The Netherlands). Eurosequence b.v. reference number is 040705/758/cc. For reference numbers of individual result-files see below. FIG. 18. A shows aliquots of the material send to Eurosequence b.v. separated in 12.5% SDS-PAGE under reducing conditions.

Proteins were N-terminally sequenced and digested by trypsin. The fragments were also sequenced to verify the correctness of the peptides. To allow fragmentation, proteins were chemically modified (see below) followed by the removal of salt and modifying agents through RP-HPLC. FIG. 18.C (HPV18-E7) show the chromatograms of the removal procedure. The profile shows 2 peaks in the first 5 minutes, corresponding to salt and chemicals eluting from the column. The main peak at min 22 represent highly purified HPV18-E7 (see also FIG. 18.A.).

Antigen and fragments were sequenced by Edman degradation with an automated sequenator (Model 494 Procise Applied Biosystems); For N-terminal sequencing 8 cycles were done.

5.3.1. N-terminal Sequencing (Eurosequence Result File 04C314)

As confirmed by N-terminal sequencing, the main sequence is 100% in agreement with the expected N-terminal sequence of the E7 protein of human papilloma virus type 18. The main sequence was: (Met)-(His)-(Gly)-(Pro)-(Lys)-(Ala)-(Thr)-(Leu). One of the minor signals at each position was brought into agreement with the n-1 mer of the protein: (His)-(Gly)-(Pro)-(Lys)-(Ala)-(Thr)-(Leu)-(Gln); this was approx. 16-20% with regard to the main sequence. The remaining minor signals were brought into agreement with the n-12 mer of the protein: (His)-(Leu)-(Glu)-(Pro)-(Gln)-(Asn)-(Glu)-(Ile); this again was approx. 16-20% with regard to the main sequence. To conclude, 60-68% of the sample are full length HPV18-E7, 16-20% are degraded at the N-terminus missing one amino acid (Met) and 16-20% are degraded at the N-terminus missing 13 amino acids (Met, His, Gly, Asp, Thr, Pro, Thr, Leu, Gln, Asp, Ile, Val, Leu). The truncated protein has a calculated WM of 10.6 kDa compared to 12.0 kDa of the full length protein. The difference in size is also seen in SDS-PAGE, FIG. 3.

Sequencing yield and result was:

| | |
|---|---|
| position: | 1 5 |
| 60 - 68% | (Met)-(His)-(Gly)-(Pro)-(Lys)-(Ala)-(Thr)-(Leu)-..................... |
| 16 - 20% | (His)-(Gly)-(Pro)-(Lys)-(Ala)-(Thr)-(Leu)-(Gln).............. |
| position: | 15 20 |
| 16 - 20% | (His)-(Leu)-(Glu)-(Pro)-(Gln)-(Asn)-(Glu)-(Ile)........................... |

Result: 100% of the sequenced protein is HPV18-E7 as confirmed by a data-base search. The protein was identified as:

| | |
|---|---|
| CAB53099 | E7 protein [Human . . . [gi: 5748509] |
| LOCUS | CAB53099 105 aa linear VRL 18- |
| | AUG-1999 |
| DEFINITION | E7 protein [Human papillomavirus type 18]. |
| ACCESSION | CAB53099 |
| VERSION | CAB53099.1 GI: 5748509 |
| DBSOURCE | embl locus HPY18493, accession Y18493.1. |
| SOURCE | Human papillomavirus type 18 |
| ORGANISM | Human papillomavirus type 18 |
| | Viruses; dsDNA viruses, no RNA stage; Papillomaviridae; |
| | Papillomavirus. |
| REFERENCE 1 | |
| AUTHORS | Laassri, M., Gul'ko, L., Vinokurova, S., Kisseljova, N., Veiko, V. and |
| | Kisseljev, F. |
| TITLE | Cloning of E6 and E7 Genes of Human Papilloma Virus Type 18 |
| | and |
| | Transformation Potential of E7 Gene and its Mutants |
| JOURNAL | Virus Genes 182, 139-149 (1999) |
| REFERENCE 2 | |
| AUTHORS | Veiko, V. P. |
| TITLE | Direct Submission |
| JOURNAL | Submitted (02-DEC-1998) V. P. Veiko, Institute of Genetics and |
| | Selection of Industrial Microorganisms, 1st Dorozhny proezd, 1, |
| | Moscow 113545, RUSSIA |

The sequence of HPV18-E7 is:

```
[SEQ ID NO:4]
                      5 10 15 20 25 30
  1 M H G P K A T L Q D I V L H L E P Q N E I P V D L L C H E Q
31 L S D S E E E N D E I D G V N H Q H L P A R R A E P Q R H T
61 M L C M C C K C E A R I E L V V E S S A D D L R A F Q Q L F
91 L K T L S F V C P W C A S Q Q
```

5.3.2. Tryptic Digest Followed by RP-HPCL (Eurosequence Result File 04E169, 04E177, 04C333, and 04E181)

Prior to trypsin digestion it was necessary to subject the sample to a reduction and pyridylethylation step as cystein residues had to be reduced and blocked to inhibit the formation of thiol-bridges. HPV18-E7 was digested by trypsin and the resulting fragments were N-terminally sequenced and subjected to a peptide profiling. From 8 theoretical cleavage products at amino acid positions 5, 52, 53, 58, 67, 71, 84 and 92 (http://www.expasy.org/tools/peptidecutter/) 7 fragments were obtained, separated by RP-HPLC and sequenced. One additional fragment that was detected derives from the N-terminally truncated version of HPV18-E7, see above. Cleavage resulted in 4 “main sequences” and 4 “minor sequences”. From additional digestion control experiments it was concluded, that the “minor signals” occurred due to some Lys- or Arg-Xaa bounds that were cleaved only poorly;

The results of the 6 sequencing cycles referred to as “main-signals” were:
cycle 1: (Met/Ala/Arg)=1^{st} amino acids of 4 fragments respectively
cycle 2: (Thr/Glu/His/Ala)=2^{nd} amino acids of 4 fragments respectively
cycle 3: (Gly/Glu/Leu/Pro)=3^{rd} amino acids of 4 fragments respectively
cycle 4: (Pro/Gin)=4^{th} amino acids of 4 fragments respectively
cycle 5: (Gin/Lys/Asp/Arg)=5^{th} amino acids of 4 fragments respectively
cycle 6: (Ile/Arg/His)=6^{th} amino acids of 4 fragments respectively

The results of the 6 sequencing cycles referred to as “minor-signals” were:
cycle 1: (His/Cys)=1^{st} amino acids of 4 fragments respectively
cycle 2: (Leu/Phe)=2^{nd} amino acids of 4 fragments respectively
cycle 3: (Pro/Met/Gln)=3^{rd} amino acids of 4 fragments respectively
cycle 4: (Arg/Asp/Leu)=4^{th} amino acids of 4 fragments respectively
cycle 5: (Cys/Val/Pro)=5^{th} amino acids of 4 fragments respectively
cycle 6: (Asn/Gin/Met/Phe)=6^{th} amino acids of 4 fragments respectively

The interpretation of the obtained fragments on the basis of known sequence information allows the following conclusion: (* minor sequences)

| | |
|---|---|
| fragment 1 (N-terminus): | (Met)-(His)-(Gly)-(Pro)-(Lys)................... |
| fragment 2 (cleaved at AA 5): | (Ala)-(Thr)-(Leu)-(Gln)-(Asp)-(Ile)............ |
| fragment 3* (N-terminus truncated): | (His)-(Leu)-(Glu)-(Pro)-(Gln)-(Asn)........... |
| fragment 4 (cleaved at AA 52): | (Arg)-(Ala)-(Glu)-(Pro)-(Gln)-(Arg)........... |
| fragment 5 (cleaved at AA 53): | (Ala)-(Glu)-(Pro)-(Gln)-(Arg)-(His)........... |
| fragment 6* (cleaved at AA 58): | (His)-(Thr)-(Met)-(Leu)-(Cys)-(Met).......... |
| fragment 7* (cleaved at AA 67): | (Cys)-(Glu)-(Ala)-(Arg)-(Ile)-(Glu)............ |
| fragment 8* (cleaved at AA 71): | (Ala)-(Phe)-(Gln)-(Gln)-(Leu)-(Phe)........... |

Taken all information together the HPV18-E7 protein was verified by sequence analysis of the underlined amino acids.

```
[SEQ ID NO:4]
                      5 10 15 20 25 30
  1 M H G P K A T L Q D I V L H L E P Q N E I P V D L L C H E Q
31 L S D S E E E N D E I D G V N H Q H L P A R R A E P Q R H T
61 M L C M C C K C E A R I E L V V E S S A D D L R A F Q Q L F
91 L K T L S F V C P W C A S Q Q
```

HPV18 E7 protein characterised in example 5.3 was used for Immunisation of goat, rabbit and mice.

5.4. Generation of Polyclonal HPV-18 E7 Antibodies in Rabbit (Chinchilla Bastard Rabbit) and Goat (Saanen Breed Goat):

Material and method was as stated in example 3 (Generation, purification, quality controls and characterization of polyclonal HPV-16 E7 antibodies from rabbit) and example 4 (Generation, purification, quality controls and characterization of polyclonal HPV-16 E7 antibodies from goat) respectively. Immunisation schedules and bleeding regimes for rabbits and goats were not altered. Slight modifications concemed the amount of administered antigen:
i.) For rabbits 100 μg purified HPV18-E7 were used per injection through out;
ii.) For goats 1 mg of HPV18-E7 was administered during the immunisation and the following 3 boosts, but was reduced to 500 μg for all further boost.

Immune response after several boost was controlled by W-blot, (C33A cell lysate as control and Hela-cell lysate as test cells respectively). Rabbit and goat serum was tested in dilutions 1:400. Blood from rabbits was processed to serum, blood from goats was processed to citrated plasma. Processed blood was stored in aliquots at −20° C. until further use. FIG. 19 shows a W-blot from rabbit serum (panel A) and a W-blot and an ELISA from goat serum (panel B). C33A lysate/HeLa lysate (125 μg/lane) was probed with pre-immune serum (pis) and serum from test-bleeding 1 to 3. The goat-ELISA shows additional samples from 2 production bleedings.

5.5. Generation of Polyclonal HPV-16 E7 and HPV18-E7 Antibodies in Mouse (Strain Balb/c):

Purified HPV16-E7 and HPV18-E7 protein was also used to raise antibodies in mice. Antibodies were generated to prove whether the antigen material described is suitable to generate polyclonal antibodies in this species. The immunisation protocol was as described in table 9.

TABLE 8
Immunisation schedule for mice

| | | |
|---|---|---|
| day 0 | Pre-immune serum | 40 μg in TGlumax ® |
| | immunisation in TGlumax ® | |
| day 14 | 1^{st} boost in TGlumax ® | 40 μg in TGlumax ® |
| day 28 | 2^{nd} boost in TGlumax ® | 40 μg in TGlumax ® |
| day 42 | 3^{rd} boost in TGlumax ® | 40 μg in TGlumax ® |
| day 50 | 4^{th} boost in TGlumax ® | 40 μg in TGlumax ® |
| day 56 | Test beeding | |

Per antigen five mice were immunized according to table 8; antisere were tested in W-blot, ELISA and IF according to example 3.5. (indirect immunofluorescence analysis). FIG. 20A shows the ELISA and the W-blot performed with sera from 5 mice immunized with purified HPV16-E7. FIG. 20B shows the ELISA and the W-blot performed with sera from 5 mice immunized with purified HPV18-E7 protein mouse serum dilution result

TABLE 9
IF experiments with mouse antisera; 18-E7 is known to be more
immuimmunogenic then 16-E7, so anti 18 sera were tested in
1:50 dilutions.

| mouse serum | dilution | result |
|---|---|---|
| 16E7 mouse L | 1:10 | + pos |
| 16E7 mouse R | 1:10 | + pos |
| 16E7 mouse O | 1:10 | +++ pos |
| 16E7 mouse RL | 1:10 | ++ pos |
| 16E7 mouse RRL | 1:10 | − neg |
| 18E7 mouse L | 1:50 | +++ pos |
| 18E7 mouse R | 1:50 | ++ pos |
| 18E7 mouse O | 1:50 | + pos |
| 18E7 mouse RL | 1:50 | ++++ pos |
| 18E7 mouse RRL | 1:50 | + pos |

For ELISA, the mouse serum was diluted 1:300 to 1:16000; purified HPV 16-E7 and 18-E7 protein was coated according to example example 4.3. (Quality control of polyclonal goat anti HPV16-E7 antiserum by titer determination ELISA), development of the plate was stopped when an O.D.₄₀₅ₙₘ of 0.700 was reached. For the W-blot mouse anti sera were tested in a 1:400 dilution. As stated above E7/2 lysate was probed with anti 16-E7 serum and HeLa lysate was probed with anti 18-E7 serum. One control lane with serum from untreated mice is shown in addition. In the IF experiments mouse sera against HPV16-E7 were tested on 16-E7 expressing cells and mouse sera against HPV18-E7 were tested on 18-E7 expressing cells (Tab. 10).

### EXAMPLE 6.1

### Proof of Cross-reactivity of Goat Polyclonal Anti HPV16-E7 and AntiHPV18-E7 Antibodies Described Herein and Combination Thereof with HPV-1, HPV-6, HPV-11, HPV-16, HPV-18, HPV-31, HPV-33, HPV-35, HPV-39, HPV-45, HPV-52, HPV56, HPV-58 and HPV-59 E7 Protein by Indirect Immunofluorescence Analysis

To appoint cross-reactivity of goat anti HPV18-E7 antibodies and of a combination of anti HPV16-E7 antibodies and anti HPV18-E7 antibodies, indirect immunofluorescence analysis was performed in U2OS cells transiently transfected with pCMV-Flag2B vectors (Stratgene) containing open-reading-frames of E7 protein of 14 different HPV genotypes according to example 3.5. (indirect immunofluorescence analysis). In these IF-experiments, anti HPV18-E7 antibodies detected E7 protein of HPV-18, -35, -39, -45 and -59 in transfected cells, whereas a combination of anti HPV16-E7 and anti HPV18-E7 antibodies recognized high risk E7 proteins of HPV16, -18, -31, -33, -35, -39, -45, -52, -56, -58 and 59. No signals were obtained from cells that had been transfected with the empty expression vector or with expression vectors expressing the low risk HPV E7 proteins 1, 6, and 11. This results clearly demonstrate that the combination of both antibodies of this invention detects all high risk HPV types mentioned above with high specificity. Under the conditions applied, no unspecific background staining was seen. A summary of the IF-results with the combination of goat anti HPV16 and 18-E7 pAB is given in Tab. 10. See also the table in example 4.9. (Test of crossreactivity of affinity purified anti-HPV-16 E7 antibodies from goat with HPV-1, HPV-6, HPV-11, HPV-18, HPV-31, HPV-33, HPV-35, HPV-39, HPV-45, HPV-52, HPV-56, HPV-58 and HPV-59 E7 protein by indirect Immunofluorescence analysis) for comparison.

TABLE 10
Crossreactivity of affinity purified anti-HPV-16 E7 and anti
HPV18-E7 antibodies from goat with E7 proteins from
HPV-1, HPV-6, HPV-11, HPV-18, HPV-31, HPV-33, HPV-35,
HPV-39, HPV-45, HPV-52, HPV-56, HPV-58 and HPV-59

| | | detection by IF: | detection by IF: |
|---|---|---|---|
| HPV | detection by IF: | goat anti HPV18- | goat anti |
| genotype | goat anti HPV16-E7 | E7 | HPV16 & 18-E7 |
| HPV1-E7 | − | − | − |
| HPV6-E7 | − | − | − |
| HPV11-E7 | − | − | − |
| HPV16-E7 | + | − | + |
| HPV18-E7 | − | + | + |
| HPV31-E7 | + | − | + |
| HPV33-E7 | + | − | + |
| HPV35-E7 | + | + | + |
| HPV39-E7 | + | + | + |
| HPV45-E7 | + | + | + |
| HPV52-E7 | + | − | + |
| HPV56-E7 | + | − | + |
| HPV58-E7 | + | − | + |
| HPV59-E7 | + | + | + |
| untransfected | − | − | − |

### EXAMPLE 6.2.

### IHC/IF with Anti HPV-18 E7 Antibody in Transient Transfected Cells and Paraffin Tissue Slices of HPV18 DNA Positive Cervical Carcinoma

According to Example 3.5 immunofluorescence experiments was done in transient transfected cells expressing HPV-18 E7 protein. In these experiments, HPV-18 E7 were detected in 5-10% of the transfected cells (FIG. 21A, panel A), whereas no signal was obtained in cells that have been transfected with the empty expression vector. In addition IHC experiments was done according to example 3.6. in paraffin embedded tissue sections of HPV-18 DNA positive cervical carcinoma biopsies. HPV-18 E7 was detected in cervical cancer cells, whereas no signal was detected in connecting fibroblast, after competition of antiHPV-18 E7 antibodies by preincubation with purified HPV-18 E7 protein and in normal squamous epithelial cells (FIG. 21A, panel B, C, D). This result clearly proves that the antibody is specific for the HPV-18 E7 protein and does not detect any non-specific background under these conditions.

### EXAMPLE 6.3.

### Cross Reactivity of the Combination of Anti HPV-16 and 18 E7 Antibodies with HPV-45 E7

With the combination of antibodies of the present invention IF experiments was done according to Example 3.5 in transient transfected cells expressing HPV-45 E7 protein. In these experiments, HPV-45 E7 were detected in 5-10% of the transfected cells (FIG. 21B, panel A), whereas no signal was obtained in cells that have been transfected with the empty expression vector. In addition IHC experiments was done according to example 3.6. in paraffin embedded tissue sections of HPV-45 DNA positive cervical carcinoma biopsies and HPV-45 DNA positive cell monolayer preparations from Pap smears (ThinPrep®, Cytyc Cooperation) that was classified as Pap V (FIG. 21B, panel B,C). In cervical cancer tissue slices HPV-45 E7 was detected in cancer cells as a clear brown staining, whereas no signal was detected in connecting fibroblast. In Pap smear preparation roughly 50% of the cells show enlarged nucleus-cytoplasm relation. Only these so-called koilocytes (cancer cells) are stained by the E7 antibodies as indicated by the brown colour but not the normal squamous epithelial cells and columnar epithelium cells (usually contained in ectocervical smears). This demonstrates that the combination of the antibodies described herein highly specific crossreact with HPV-45 E7 protein and does not detect any non-specific background under these conditions.

### EXAMPLE 7

### Comparison of Antibodies of the Present Invention and Commercially Available Anti HPV-16 E7 and HPV-18 E7 Antibodies

With the antibodies of the present invention and commercially available antibodies against the HPV16- or HPV18-E7 proteins, several test were carried out. Commercially available antibodies were purchased from Santa Cruz Biotechnology, Inc (European Support Office Bergheimer Str. 89-269115 Heidelberg, Germany), HyTest Ltd (Pharmacity, 5th floor Itainen Pitkakatu 4C, 20520 Turku, Finland ), Zymed (Zymed Laboratories, Inc. 561 Eccles Avenue South San Francisco, Calif. 94080), RDI (Research Diagnostics Inc. Pleasant Hill Road Flanders N.J. 07836 USA) and BioCarta (BioCarta Europe; Borsteler Chaussee 53; Hamburg, Germany). The monoclonal mouse anti HPV16-E7 antibody DP 18 from EMD (EMD Biosciences, Inc. 10394 Pacific Center Court, San Diego Calif.) was intended to be tested in addition, but could not be purchased due to quality-problems from producer-side at the time.

The Santa Cruz antibody ED17, the Zymed antibody clone 8C9 and the RDI anti 16-E7 antibody are mABs from mice, generated against full length HPV 16-E7 protein. Santa Cruz antibody N19 is a pAB from goat, generated against an N-teminal peptide of HPV18-E7; HyTest and RDI antibodies against HPV18-E7 are mouse mAB against full length HPV18-E7 protein respectively.

Monoclonal antibodies from BioCarta against HPV16-L1 and against HPV1, 6, 11, 16, 18, and 31-L1 have been included as control antibodies in W-blot and ELISA experiment. As they showed no reactivity with HPV16- and 18-E7 protein, they were not tested further.

TABLE 11

| Antibody | Katalog Code | Lot.-Nr. |
|---|---|---|
| Santa Crux ED17 | cs-6981 | # J3103 |
| Santa Crux N-19 | Sc-1590 | # B2504 |
| Hytest | 3HP18, colne718-85 | # 04/02-HP18-85 |
| Zymed | NO. 28-0006 | # 40286557 |
| RDI | RDI-TRK3HP1-325 | # 04/03-HP1-325a |
| RDI | RDI-TRK3HP18-718 | # 04/01-HP18-67 |

### EXAMPLE 7.1.

### ELISA and Western

All antibodies detected the antigen they were made against in W-blots. Cell lines expressing either HPV16-E7 (E7/2) or HPV18-E7 (HeLa) were used as samples. Specificity of all antibodies was comparable although occasionally additional bands were detected. No cross reactivity between anti 16-E7 antibodies and 18-E7 protein or anti 18-E7 antibodies and 16-E7 protein was found. Signal strength varied between the antibodies (See FIG. 23A)

For ELISA experiments monoclonals were tested in dilutions 1:25.000 to 1:1000000, and polyclonals were tested in dilutions 1:2500-1:100000. Again, all mAB detected their specific antigen similarly well—no cross reactivity was observed. Sensitivity varied strongly between mABs. Sensitivity of pAB was comparable, the antigen that was used to generate the respect antibody was detected and no significant cross reactivity was seen with the purchased pAB N19. In contrast, antibodies anti 16-E7 and anti 18-E7 of the present invention showed moderate cross reactivity; i.e. anti 16-E7 pAB gave weak signals with 18-E7 proteins, and anti 18-E7 pAB gave weak signals with 16-E7. proteins (FIG. 23A).

### EXAMPLE 7.2.

### Immunofluorescence and Immunohistochemistry

Like antibodies of the present invention against HPV16-E7 and HPV18-E7 respectively also antibodies from other suppliers were tested in immunofluorescence according to example 3.5. (indirect immunofluorescence analysis) As described, U2OS cells were transiently transfected with pCMV-Flag2B vectors (Stratagene) containing open-reading-frames of HPV 16, 18, 31, 33, 35, 39, 45, 52, 56, 58 or 59 respectively. Results from IF-experiments are shown in FIG. 22. Zymed antibodies against HPV16-E7 (clone 8C9) and Santa Cruz antibodies against HPV16-E7 (ED17) were tested again, but in less diluted concentrations compared to the experimental setup described in example 3.5. FIG. 22 shows a conclusive summery of IF-results from all antibodies tested. Antibodies that gave a weak signal or a signal just above background are marked with an empty circle, (◯) antibodies that gave a strong clear signal are marked with a filled circle(●). In this experiments no one of the antibodies from other suppliers was able to detect HPV-31 E7 protein.

For IHC, antibodies were tested on paraffin section of HPV-16, -18 or -45 DNA positive tumours as described in example 3.6. For all antibodies a test protocol was used that had been optimised for goat anti 16-E7 and anti 18-E7 antibodies described in the present invention respectively. Results for IHC testing are summarised in FIG. 23B. In this experiments no one of the commercially available antibodies was able to detect HPV-45 E7 protein in HPV-45 DNA positive tumors whereas antibodies of the present invention gave a strong and specific signalal (See FIG. 23B)

### EXAMPLE 8

### Proof of AntiHPV-45 E7 Antibody from Goat

For the production of an antiHPV-45 E7 antibody HPV-45 E7 protein was expressed and purified according Example 1 and 2 and used for generation of polyclonal goat antibodies according to example 4.1. This antibodies was purified (example 4.4) and detection of high risk HPV E7 proteins was tested in IF experiments according to example 3.5. In these IF-experiments, anti HPV-45 E7 antibody detected E7 protein of HPV-18, -45 and -59 in transfected cells but could not recognize E7 protein of HPV-16, -31, -33, -35, -39, -52, -56 and -58.

TABBLE 12
Crossreactivity of affinity purified antiHPV-45 E7 antibody from
goat with E7 proteins from HPV-16, HPV-18, HPV-31, HPV-33,
HPV-35, HPV-39, HPV-45, HPV-52, HPV-56, HPV-58 and HPV-59.

| antiHPV-45 E7 antibody | HPV-Genotypes |
|---|---|
| detection | 45, 18, 59 (weak) |
| No detection | 16, 31, 33, 35, 39, 52, 56, 58 |

### ADDITIONAL REFERENCES

Alexander and Phelps (2000) Expert Opin. Investig. Drugs 9: 1753-65

Bringold, (2000) Exp Gerontol 35:317-29

Clavel, (1999) Br J Cancer 80:1306-11

Francis, (2000) J Virol 74:2679-86

Fujikawa, (1994) Virology 204: 789-793

Giarre, (2001) J Virol 75:4705-12

Howley, (1996) Fields Virology, 3rd edn. Lipppincott-Raven, Philadelphia, Pa., pp. 2045-2076.

Klaes, (2001) Int J Cancer 92:276-84

Koss, (1989) J. Am. Med. Assoc. 261: 737-743

Larsen, N. S. (1994) J. Natl. Cancer Inst. 86: 6-7

McMurray, (2001) Int J Exp Pathol 82:15-33

Meijer, (2000) J Clin Pathol 53:41-3

Milde-Langosch, (2000) Virchows Arch 437:227-33

Milde-Langosch, (1999) Pathologe 20:15-24

Mantovani, (2001) Oncogene 20:7874-87

Münger, (2001) Oncogene 20:7888-98

Papanicolaou, (1942) Science 95: 438-439

Renshaw, (2001) Cancer 93:106-10

Sato, (1989) Virology: 170: 311-314

Schiffman, (1995) Cancer 76:1888-1901

Schwarz, (1985) Nature 314:111-114

Selvey (1992) J Virol Methods 37: 119-127

Stacey, (1994) Oncogene 9: 635-645

Studier, (1986) J Mol Biol 189:113-30

Tindle, (1990) Peptide Res 3:162-166

Trofatter, (1997) Jr. Am J Med 102:21-7

Serrano, (1997) Exp Cell Res 237:7-13

von Knebel Doeberitz, (2001) Dis Markers 17:123-8

Walboomers, (1995) J Clin Pathol 48:728-32

Walboomers, (1999) J Pathol 189:12-9

Zatsepina, (1997) Oncogene 14:1137-45

zur Hausen, (2000) J. Natl. Cancer Inst 92: 690-8

1. A combination of antibodies comprising
(a) an anti-HPV-16 E7 antibody obtainable by
(i) eliciting an in vivo humoral response against HPV-16 E7 protein or a fragment thereof in a goat; and
(ii) affinity-purifying antibodies as obtained in the eliciting-step (i); and
(b) an anti-HPV-18 E7 antibody.

2. The combination of antibodies of claim 1, wherein said HPV-16 E7 protein or a fragment thereof is recombinantly produced.

3. The combination of antibodies of claim 1, wherein said HPV-16 E7 protein or said fragment thereof is expressed in E. coli.

4. The combination of antibodies of claim 1 wherein said HPV-16 E7 protein or said fragment thereof is highly purified.

5. The combination of antibodies of claim 4, wherein said highly purified HPV-16 E7 protein or a fragment thereof is purified by a combination of ion exchange chromatography and gel filtration.

6. The combination of antibodies of claim 5, wherein said purification further comprises, prior to ion exchange chromatography and gel filtration, a protein precipitation step.

7. The combination of antibodies of claim 1, wherein said affinity purification of the obtained antibodies is carried out over immobilized HPV-16 E7 protein or a fragment thereof.

8. The combination of antibodies of claim 7, wherein said HPV-16 E7 protein or a fragment thereof is immobilized on PVDF membranes, nitrocellulose, sepharose, agarose, DEAE-cellulose or DEAE.

9. The combination of antibodies of claim 1, wherein said anti-HPV-18 E7 antibody is a polyclonal or monoclonal antibody.

10. The combination of antibodies of claim 9, wherein said anti-HPV-18 E7 polyclonal antibody is derived from a non-human animal selected from the group consisting of rat, mouse, guinea pig, chicken, duck, sheep, horse, goat, pig, cattle and donkey.

11. The combination of antibodies of claim 9, wherein said anti-HPV-18 E7 polyclonal antibody is obtainable by
(i) eliciting an in vivo humoral response against highly purified HPV-16 E7 protein or a fragment thereof in a rabbit; and
(ii) affinity-purifying antibodies as obtained in the eliciting-step (i).

12-15. (canceled)

16. A method for the preparation of a diagnostic composition comprising the step of formulating the combination of antibodies of claim 1 with a diagnostically acceptable carrier, diluent, buffer, or storage solution.

17. The method of claim 16, wherein said diagnostic composition further comprises suitable means for detection.

18. A diagnostic composition comprising the combination of antibodies of claim 1.

19. A kit comprising the combination of antibodies of claim 1.

20. An in vitro method for the detection of high risk HPV E7 protein comprising the steps of
incubating a biological sample with the combination of antibodies of claim 1; and
measuring and/or detecting E7 protein of high risk HPV,
whereby the presence, the absence or the amount of specifically-bound antibodies of the combination of antibodies of claim 1 is indicative for the presence of high risk HPV E7 protein.

21. The method of claim 20, wherein said high risk HPV is HPV-16, HPV-18, HPV-31, HPV-33, HPV-35, HPV-39, HPV-45, HPV-52, HPV-56, HPV-58 and/or HPV-59.

22. The method of claim 20, wherein the detection of high risk HPV E7 protein is used for determining the occurrence of a sexually transmittable disease or cancer.

23. The method of claim 20 further comprising a further step (c), whereby in said step (c) the presence, the absence or the amount of specifically- bound antibodies of the combination of antibodies of claim 1 of step (b) is compared to the presence, the absence or the amount of specifically-bound antibodies of the combination of antibodies of claim 1 in a negative or a positive control sample.

24. (canceled)

25. The method of claim 22 wherein said sexually transmitted disease is a high risk HPV infection or wherein said cancer is cervical cancer, breast cancer/mamma cancer, prostate cancer, head and neck cancer, penile cancer and/or anogenital cancer/neoplasia (AIN).

26. A method for the production of a combination of an anti-HPV-16 E7 antibody and an anti-HPV-18 E7 antibody comprising the steps of
(a) eliciting an in vivo humoral response against HPV-16 E7 protein or a fragment thereof in a goat;
affinity-purifying antibodies as obtained in the eliciting-step (a) and
mixing the antibody of step (b) with an anti-HPV-18 E7 antibody.

27. A method for the production of a combination of an anti-HPV-16 E7 antibody and an anti-HPV-18 E7 antibody comprising the steps of
(a) eliciting an in vivo humoral response against HPV-16 E7 protein or a fragment thereof and against HPV-18 E7 protein or a fragment thereof in a goat; and
(b) affinity-purifying antibodies as obtained in the eliciting-step (a).

28. The method of claim 25 or 26, wherein said HPV-16 E7 protein or fragment thereof is highly-purified.

29. The combination of antibodies of claim 1, wherein said highly purified HPV-16 E7 protein or said fragment thereof is a native, highly purified HPV-16 E7 protein or a fragment thereof.

30. A diagnostic composition comprising the antibody obtainable as described in step (a) of claim 1.

31. A method for the preparation of a diagnostic composition comprising the step of formulating the antibody of claim 30 with a diagnostically acceptable carrier, diluent, buffer, or storage solution.

32-36. (canceled)

37. A kit comprising the antibody obtainable as described in step (a) of claim 1.

38. An in vitro method for the detection of HPV-31, HPV-33, HPV-35, HPV-39, HPV-45, HPV-52, HPV-56, HPV-58 and/or HPV-59 E7 protein comprising the steps of
a) incubating a biological sample with the antibody obtainable as described in step (a) of claim 1 or the antibody combination of claim 1; and
measuring and/or detecting E7 protein of HPV-3 1, HPV-33, HPV-35, HPV-39, HPV-45, HPV-52, HPV-56, HPV-58 and/or HPV-59,
whereby the presence, the absence or the amount of specifically-bound said antibodies is indicative for the presence of high risk HPV E7 protein.

39. A diagnostic composition comprising the combination of antibodies obtained by the method of claim 16.

40. A diagnostic composition comprising the combination of antibodies obtained by the method of claim 17.

41. A kit comprising the diagnostic composition of claim 18.

42. The method of claim 26, wherein said HPV-16 E7 protein or fragment thereof is highly-purified.

43. A kit comprising the diagnostic composition of claim 30.


SEQUENCE LISTING (23 sequences)

SEQ ID NO: 1 - DNA, 297 bp, Human papillomavirus
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 2 - protein, 98 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 3 - DNA, 318 bp, Human papillomavirus
atgcatggacctaaggcaacattgcaagacattgtattgcatttagagcctcaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagctagaattgagctagtagtagaaagctcagcagacgaccttcgagcattccagcagctgtttctgaaaaccctgtcctttgtgtgtccgtggtgtgcatcccagcagtaa

SEQ ID NO: 4 - protein, 105 aa, Human papillomavirus
MHGPKATLQDIVLHLEPQNEIPVDLLCHEQLSDSEEENDEIDGVNHQHLPARRAEPQRHTMLCMCCKCEARIELVVESSADDLRAFQQLFLKTLSFVCPWCASQQ

SEQ ID NO: 5 - protein, 8 aa, Artificial Sequence
  Description of Artificial Sequence Synthetic peptide
MHGDTPTL

SEQ ID NO: 6 - protein, 8 aa, Artificial Sequence
  Description of Artificial Sequence Synthetic peptide
GDTPTLHE

SEQ ID NO: 7 - protein, 8 aa, Artificial Sequence
  Description of Artificial Sequence Synthetic peptide
HGDTPTLH

SEQ ID NO: 8 - protein, 6 aa, Artificial Sequence
  Description of Artificial Sequence Synthetic peptide
MHGDTP

SEQ ID NO: 9 - protein, 6 aa, Artificial Sequence
  Description of Artificial Sequence Synthetic peptide
AHYNIV

SEQ ID NO: 10 - protein, 6 aa, Artificial Sequence
  Description of Artificial Sequence Synthetic peptide
CDSTLR

SEQ ID NO: 11 - protein, 6 aa, Artificial Sequence
  Description of Artificial Sequence Synthetic peptide
LCVQST

SEQ ID NO: 12 - protein, 6 aa, Artificial Sequence
  Description of Artificial Sequence Synthetic peptide
TLEDLL

SEQ ID NO: 13 - protein, 8 aa, Artificial Sequence
  Description of Artificial Sequence Synthetic peptide
MHGPKATL

SEQ ID NO: 14 - protein, 8 aa, Artificial Sequence
  Description of Artificial Sequence Synthetic peptide
HGPKATLQ

SEQ ID NO: 15 - protein, 8 aa, Artificial Sequence
  Description of Artificial Sequence Synthetic peptide
HLEPQNEI

SEQ ID NO: 16 - protein, 5 aa, Artificial Sequence
  Description of Artificial Sequence Synthetic peptide
MHGPK

SEQ ID NO: 17 - protein, 6 aa, Artificial Sequence
  Description of Artificial Sequence Synthetic peptide
ATLQDI

SEQ ID NO: 18 - protein, 6 aa, Artificial Sequence
  Description of Artificial Sequence Synthetic peptide
HLEPQN

SEQ ID NO: 19 - protein, 6 aa, Artificial Sequence
  Description of Artificial Sequence Synthetic peptide
RAEPQR

SEQ ID NO: 20 - protein, 6 aa, Artificial Sequence
  Description of Artificial Sequence Synthetic peptide
AEPQRH

SEQ ID NO: 21 - protein, 6 aa, Artificial Sequence
  Description of Artificial Sequence Synthetic peptide
HTMLCM

SEQ ID NO: 22 - protein, 6 aa, Artificial Sequence
  Description of Artificial Sequence Synthetic peptide
CEARIE

SEQ ID NO: 23 - protein, 6 aa, Artificial Sequence
  Description of Artificial Sequence Synthetic peptide
AFQQLF